(19) United States
(12) Patent Application Publication
(10) Pub. No.: US 2009/0324510 A1
(43) Pub. Date: Dec. 31, 2009

(54) DRUG COMBINATIONS FOR THE TREATMENT OF RESPIRATORY TRACT DISEASES

(75) Inventors: Ingo Konetzki, Warthausen (DE); Thierry Bouyssou, Warthausen (DE); Sabine Pestel, Attenweiler (DE); Andreas Schnapp, Biberach (DE)

Correspondence Address: MICHAEL P. MORRIS;BOEHRINGER INGELHEIM USA CORPORATION, 900 RIDGEBURY RD, P O BOX 368, RIDGEFIELD, CT, 06877-0368, US

(73) Assignee: BOEHRINGER INGELHEIM INTERNATIONAL GMBH, Ingelheim (DE)

(21) Appl. No.: 12/376,232

(22) PCT Filed: Aug. 3, 2007
(86) PCT No.: PCT/EP07/58050

§ 371 Date: Mar. 30, 2009

(30) Foreign Application Priority Data
Aug. 7, 2006 (EP) 06118527.8

(51) Int. Cl. A61K 9/12; A61K 31/538; A61K 31/423; A61P 11/00

(52) U.S. Cl. 424/45; 514/230.5; 514/375; 424/43

(57) ABSTRACT

The present invention relates to novel drug combinations which, besides one or more, preferably on compound of the general formula (1), wherein remainder n, A, R1, R2, and R3 can have the meanings given in the claims and in the description comprise at least one further active ingredient 2 and method for the production thereof and the use thereof as drugs.

The present invention relates to new pharmaceutical combinations, which contain at least one further active substance 2 in addition to one or more, preferably one compound of general formula 1

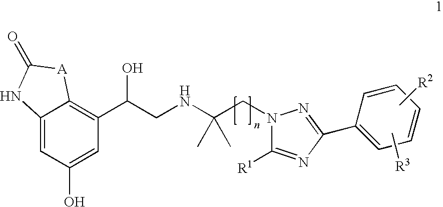

wherein the groups n, A, R¹, R² and R³ may have the meanings given in the claims and specification, processes for preparing them and their use as medicaments.

### DETAILED DESCRIPTION OF THE INVENTION

The present invention relates to pharmaceutical combinations which contain in addition to one or more, preferably one compound of general formula 1

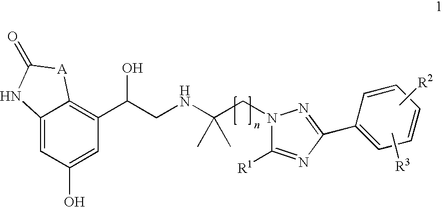

wherein
n denotes 1, 2, 3 or 4;
A denotes a double-bonded group selected from among O, S, NR⁶, CR⁴R⁵—S CR⁴R⁵—O, CR⁴R⁵—NR⁶, CH═CH or CH₂—CH₂;
R¹ denotes C₁₋₆-alkyl;
R² and R³ which may be identical or different denote H, C₁₋₆-alkyl, C₂₋₆-alkenyl, C₂₋₆-alkynyl, C₃₋₆-cycloalkyl, C₁₋₆-haloalkyl, O—C₁₋₆-haloalkyl, halogen, OH, CN, NO₂, O—C₁₋₆-alkyl, C₂₋₆-alkyl-OH, NH₂, NH—C₁₋₆-alkyl, N(C₁₋₆-alkyl)₂, NHCO—C₁₋₆-alkyl, NHSO₂—C₁₋₆-alkyl, S—C₁₋₆-alkyl, SO—C₁₋₆-alkyl, SO₂—C₁₋₆-alkyl, SO₂NH₂, SO₂NH—C₁₋₆-alkyl, SO₂N(C₁₋₆-alkyl)₂, CONH₂, CONH—C₁₋₆-alkyl, CON(C₁₋₆-alkyl)₂, CO—C₁₋₆-alkyl, COOH or COO—C₁₋₄alkyl, or
R² and R³ together denote a 2-bonded group selected from O—CR⁴R⁵—O, O—CR⁴R⁵—NR⁶ or CH═CH—CH═CH;
R⁴ denotes H or C₁₋₆-alkyl;
R⁵ denotes H or C₁₋₆-alkyl;
R⁶ denotes H or C₁₋₆-alkyl;
   optionally in the form of the pharmaceutically acceptable acid addition salts, hydrates or solvates thereof, contain at least one further active substance 2.

Preferably the present invention relates to pharmaceutical combinations which contain in addition to one or more, preferably one compound of formula 1 as a further active substance 2 one or more compounds which are selected from among the categories of the anticholinergics (2a), PDE-IV-inhibitors (2b), steroids (2c), LTD4-antagonists (2d) and EGFR-inhibitors (2e).

Preferred pharmaceutical combinations as defined above are those which contain in addition to one or more, preferably one compound of general formula 1, wherein
n denotes 1, 2 or 3, preferably 2;
A denotes a double-bonded group selected from among CR⁴R⁵—O, CH═CH or CH₂—CH₂, preferably CR⁴R⁵—O;
R¹ denotes C₁₋₄-alkyl;
R² and R³ which may be identical or different denote H, C₁₋₄-alkyl, C₂₋₄-alkenyl, C₂₋₄-alkynyl, C₃₋₆-cycloalkyl, C₁₋₄-haloalkyl, O—C₁₋₄-haloalkyl, halogen, OH, CN, NO₂, C₂₋₄-alkyl-OH, O—C₁₋₄-alkyl, COOH or COO—C₁₋₄-alkyl, or
R² and R³ together denote a 2-bonded group selected from O—CR⁴R⁵—O, O—CR⁴R⁵—NR⁶ or CH═CH—CH═CH;
R⁴ denotes H or C₁₋₄-alkyl;
R⁵ denotes H or C₁₋₄-alkyl;
R⁶ denotes H or C₁₋₄-alkyl,
   optionally in the form of the pharmaceutically acceptable acid addition salts, hydrates or solvates thereof, at least one further active substance 2.

Preferred pharmaceutical combinations as defined above are those which contain in addition to one or more, preferably one compound of general formula 1, wherein
A denotes a double-bonded group selected from among CR⁴R⁵—O, CH═CH or CH₂—CH₂, preferably CR⁴R⁵—O with
R⁴ denotes H, methyl, ethyl, preferably H or methyl, particularly preferably H;
R⁵ denotes H, methyl, ethyl, preferably H or methyl, particularly preferably H;
   and wherein n, R¹, R², R³ and R⁶ in each case may have one of the meanings given above or hereinafter, optionally in the form of the pharmaceutically acceptable acid addition salts, hydrates or solvates thereof, at least one further active substance 2.

Preferred pharmaceutical combinations as defined above are those which contain in addition to one or more, preferably one compound of general formula 1, wherein R¹ denotes methyl, ethyl or propyl, preferably methyl or ethyl, particularly preferably methyl and wherein n, A, R², R³, R⁴, R⁵ and R⁶ in each case may have one of the meanings given above or hereinafter, optionally in the form of the pharmaceutically acceptable acid addition salts, hydrates or solvates thereof, at least one further active substance 2.

Preferred pharmaceutical combinations as defined above are those which contain in addition to one or more, preferably one compound of general formula 1, wherein
R² denotes H, methyl, ethyl, propyl, vinyl, allyl, propargyl, cyclopropyl, cyclopentyl, cyclohexyl, CH₂Cl, CHCl₂, CCl₃, CH₂F, CHF₂, CF₃, CH₂—CH₂Cl, CH₂—CHCl₂, CH₂—CCl₃, CH₂—CH₂F, CH₂—CHF₂, CH₂—CF₃, CH₂—CH₂OH, fluorine, chlorine, bromine, OH, CN, NO₂, methoxy, ethoxy, propoxy, COOH, COO-methyl, COO-ethyl, COO-propyl or COO-butyl;
R³ denotes methyl, ethyl, propyl, vinyl, allyl, propargyl, cyclopropyl, cyclopentyl, cyclohexyl, CH₂Cl, CHCl₂, CCl₃, CH₂F, CHF₂, CF₃, CH₂—CH₂Cl, CH₂—CHCl₂, CH₂—CCl₃, CH₂—CH₂F, CH₂—CHF₂, CH₂—CF₃, CH₂—CH₂OH, fluorine, chlorine, bromine, OH, CN, NO₂, methoxy, ethoxy, propoxy, COOH, COO-methyl, COO-ethyl, COO-propyl or COO-butyl, or
R² and R³ together denote a 2-bonded group selected from O—CR⁴R⁵—O, O—CR⁴R⁵—NR⁶ or CH═CH—CH═CH;
R⁴ denotes H, methyl, ethyl, preferably H or methyl, particularly preferably H;
R⁵ denotes H, methyl, ethyl, preferably H or methyl, particularly preferably H;
R⁶ denotes H, methyl, ethyl, preferably H or methyl, particularly preferably H;
   and wherein n, A and R¹ in each case may have one of the meanings given above or hereinafter, optionally in the form of the pharmaceutically acceptable acid addition salts, hydrates or solvates thereof, at least one further active substance 2.

Preferred pharmaceutical combinations as defined above are those which contain in addition to one or more, preferably one compound of general formula 1, wherein
R² denotes H, methyl, ethyl, CF₃, CH₂—CF₃, fluorine, chlorine, OH, methoxy, ethoxy, COOH or COO-methyl;
R³ denotes methyl, ethyl, propyl, vinyl, allyl, cyclopropyl, cyclopentyl, cyclohexyl, CH₂F, CHF₂, CF₃, CH₂—CH₂F, CH₂—CHF₂, CH₂—CF₃, CH₂—CH₂OH, fluorine, chlorine, OH, CN, methoxy, ethoxy, COOH, COO-methyl, COO-ethyl or COO-butyl, or
R² and R³ together denote a 2-bonded group selected from O—CH₂—O, O—CMe₂—O or CH═CH—CH═CH;
   and wherein n, A and R¹ in each case may have one of the meanings given above or hereinafter, optionally in the form of the pharmaceutically acceptable acid addition salts, hydrates or solvates thereof, at least one further active substance 2.

Preferred pharmaceutical combinations as defined above are those which contain in addition to one or more, preferably one compound of general formula 1, wherein
R² denotes H, methyl, ethyl, CF₃, fluorine, chlorine, OH or methoxy;
R³ denotes methyl, ethyl, cyclopropyl, cyclohexyl, CF₃, fluorine, chlorine, OH, CN, methoxy, ethoxy, COOH, COO-methyl, COO-ethyl or COO-butyl, or
R² and R³ together denote a 2-bonded group selected from O—CH₂—O or CH═CH—CH═CH;
   and wherein n, A and R¹ in each case may have one of the meanings given above or hereinafter, optionally in the form of the pharmaceutically acceptable acid addition salts, hydrates or solvates thereof, at least one further active substance 2.

Preferred pharmaceutical combinations as defined above are those which contain in addition to one or more, preferably one compound of general formula 1, wherein
R² denotes H, methyl, fluorine, chlorine, OH or methoxy;
R³ denotes methyl, ethyl, CF₃, fluorine, chlorine, OH, methoxy, ethoxy, COOH, COO-methyl or COO-butyl, or
R² and R³ together denote a 2-bonded group selected from O—CH₂—O or CH═CH—CH═CH, preferably O—CH₂—O;
   and wherein n, A and R¹ in each case may have one of the meanings given above or hereinafter, optionally in the form of the pharmaceutically acceptable acid addition salts, hydrates or solvates thereof, at least one further active substance 2.

Preferred pharmaceutical combinations as defined above are those which contain in addition to one or more, preferably one compound of general formula 1, wherein R² denotes hydrogen and wherein n, A, R¹ and R³ in each case may have one of the meanings given above or hereinafter, optionally in the form of the pharmaceutically acceptable acid addition salts, hydrates or solvates thereof, at least one further active substance 2.

Preferred pharmaceutical combinations as defined above are those which contain in addition to one or more, preferably one compound of general formula 1, wherein n denotes 2 and wherein A, R¹, R² and R³ in each case may have one of the meanings given above or hereinafter, optionally in the form of the pharmaceutically acceptable acid addition salts, hydrates or solvates thereof, at least one further active substance 2.

Preferred pharmaceutical combinations as defined above are those which contain in addition to one or more, preferably one compound of general formula 1, wherein
R³ denotes methyl, ethyl, CF₃, fluorine, chlorine, OH, methoxy, ethoxy, COOH, COO-methyl or COO-butyl, preferably methyl, CF₃, fluorine, chlorine, OH, methoxy, COOH or COO-methyl, particularly preferably methyl, CF₃, fluorine, chlorine, methoxy or COOH
   and wherein n, A, R¹ and R² in each case may have one of the meanings given above or hereinafter, optionally in the form of the pharmaceutically acceptable acid addition salts, hydrates or solvates thereof, at least one further active substance 2.

In another aspect the present invention relates to the above-mentioned new compounds of formula 1 in the form of the individual optical isomers, mixtures of the individual enantiomers or racemates. Particularly preferred are compounds of formula 1 in the form of the enantiomerically pure compounds, while the R-enantiomers of the compounds of formula 1 according to the invention are of exceptional importance. The R-enantiomers of the compounds of formula 1 can be represented by general formula R-1

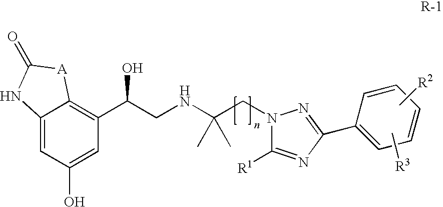

wherein the groups R¹, R² and R³ may have the meanings given above. Methods of separating racemates into the respective enantiomers are known in the prior art and may be used to prepare the enantiomerically pure R- and S-enantiomers of the compounds of formula 1 in analogous manner.

As mentioned above, the compounds of formula 1 may be converted into the salts thereof, particularly, for pharmaceutical use, into the physiologically and pharmacologically acceptable salts thereof. These salts may be present on the one hand as physiologically and pharmacologically acceptable acid addition salts of the compounds of formula 1 with inorganic or organic acids. The acid addition salts may be prepared for example from hydrochloric acid, hydrobromic acid, sulphuric acid, phosphoric acid, methanesulphonic acid, acetic acid, fumaric acid, succinic acid, lactic acid, citric acid, tartaric acid or maleic acid. In addition, mixtures of the above-mentioned acids may also be used. For preparing the alkali and alkaline earth metal salts of the compound of formula 1 the alkali and alkaline earth metal hydroxides and hydrides are preferred, the hydroxides and hydrides of the alkali metals, particularly sodium and potassium, are preferred, sodium and potassium hydroxide being particularly preferred.

Optionally the compounds of general formula 1 may be converted into the salts thereof, particularly, for pharmaceutical use, into the pharmacologically acceptable acid addition salts thereof with an inorganic or organic acid. Suitable acids for this purpose include for example succinic acid, hydrobromic acid, acetic acid, fumaric acid, maleic acid, methanesulphonic acid, lactic acid, phosphoric acid, hydrochloric acid, sulphuric acid, tartaric acid or citric acid. It is also possible to use mixtures of the above-mentioned acids.

The invention relates to the compounds in question, optionally in the form of the individual optical isomers, mixtures of the individual enantiomers or racemates, in the form of the tautomers as well as in the form of the free bases or the corresponding acid addition salts with pharmacologically acceptable acids—such as for example acid addition salts with hydrohalic acids—for example hydrochloric or hydrobromic acid—or organic acids—such as for example oxalic, fumaric, diglycolic or methanesulphonic acid.

Preferred pharmaceutical combinations as defined above are those which contain, in addition to one or more, preferably one compound of general formula 1, wherein A denotes CH₂—O and wherein the groups n, R¹, R² and R³ may have the meanings given above, optionally in the form of the pharmaceutically acceptable acid addition salts, hydrates or solvates thereof, at least one further active substance 2.

Of the compounds of formula 1 wherein A denotes CH₂—O, the preferred regioisomers are those characterised by general formula 1.1.

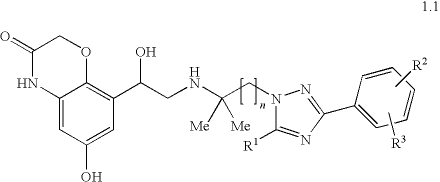

In a preferred aspect the present invention relates to the above pharmaceutical combinations which contain, in addition to one or more, preferably one compound of general formula 1.1 wherein n, R¹, R² and R³ may have the meanings given above, optionally in the form of the pharmaceutically acceptable acid addition salts, hydrates or solvates thereof, at least one further active substance 2. Particularly preferred are the R-enantiomers of the compounds of formula 1.1.

Compounds of formula 1 wherein A denotes CH═CH are characterised by general formula 1.2.

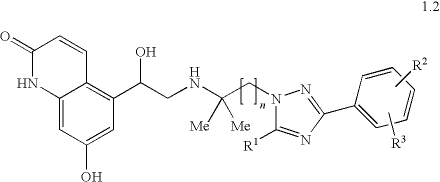

In a preferred aspect the present invention relates to the above pharmaceutical combinations which contain, in addition to one or more, preferably one compound of general formula 1.2 wherein n, R¹, R² and R³ may have the meanings given above, optionally in the form of the pharmaceutically acceptable acid addition salts, hydrates or solvates thereof, at least one further active substance 2. Particularly preferred are the R-enantiomers of the compounds of formula 1.2.

Compounds of formula 1 wherein A denotes CH₂—CH₂ are characterised by general formula 1.3.

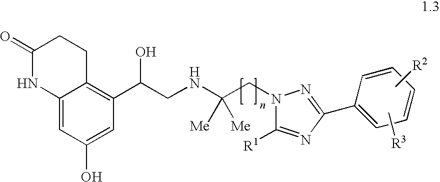

In a preferred aspect the present invention relates to the above pharmaceutical combinations which contain, in addition to one or more, preferably one compound of general formula 1.3 wherein n, R¹, R² and R³ may have the meanings given above, optionally in the form of the pharmaceutically acceptable acid addition salts, hydrates or solvates thereof, at least one further active substance 2. Particularly preferred are the R-enantiomers of the compounds of formula 1.3.

Of the compounds of formula 1, wherein A denotes CR⁴R⁵—O and R⁴ or R⁵ denotes methyl, the preferred regioisomers are those which are characterised by general formula 1.4.

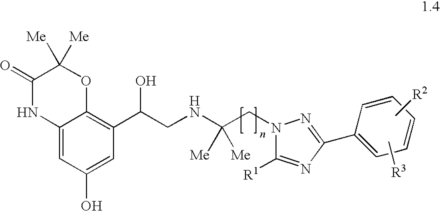

In a preferred aspect the present invention relates to the above pharmaceutical combinations which contain, in addition to one or more, preferably one compound of general formula 1.4 wherein n, R¹, R² and R³ may have the meanings given above, optionally in the form of the pharmaceutically acceptable acid addition salts, hydrates or solvates thereof, at least one further active substance 2. Particularly preferred are the R-enantiomers of the compounds of formula 1.4.

Preferred pharmaceutical combinations as defined above are those which contain, in addition to one or more, preferably one compound of general formula 1, wherein A denotes a double-bonded group selected from among O, CR⁴R⁵ or NR⁶ and wherein n, R¹, R², R³, R⁴, R⁵ and R⁶ in each case may have one of the meanings given above or hereinafter, optionally in the form of the pharmaceutically acceptable acid addition salts, hydrates or solvates thereof, at least one further active substance 2.

Preferred pharmaceutical combinations as defined above are those which contain, in addition to one or more, preferably one compound of general formula 1, wherein A denotes a double-bonded group S and wherein n, R¹, R², and R³ in each case may have one of the meanings given above or hereinafter, optionally in the form of the pharmaceutically acceptable acid addition salts, hydrates or solvates thereof, at least one further active substance 2.

Particularly preferred are pharmaceutical combinations which contain in addition to one or more, preferably one compound of general formula 1 selected from among the compounds

1.1: 8-(2-{3-[3-(4-fluoro-phenyl)-5-methyl-[1,2,4]triazol-1-yl]-1.1-dimethyl-propylamino}-1-hydroxy-ethyl)-6-hydroxy-4H-benzo[1,4]oxazin-3-one;

1.2: 8-{2-[1,1-dimethyl-3-(5-methyl-3-p-tolyl-[1,2,4]triazol-1yl)-propylamino]-1-hydroxy-ethyl}-6-hydroxy-4H-benzo[1,4]oxazin-3-one;

1.3: 8-(2-{1,1-dimethyl-3-[5-methyl-3-(4-trifluoromethyl-phenyl)-[1,2,4]triazol-1-yl]-propylamino}-1-hydroxy-ethyl)-6-hydroxy-4H-benzo[1,4]oxazin-3-one;

1.4: 8-(2-{3-[3-(3,5-difluoro-phenyl)-5-methyl-[1,2,4]triazol-1-yl]-1,1-dimethyl-propylamino}-1-hydroxy-ethyl)-6-hydroxy-4H-benzo[1,4]oxazin-3-one;

1.5: 3-(1-{3-[2-hydroxy-2-(6-hydroxy-3-oxo-3,4-dihydro-2H-benzo[1,4]oxazin-8-yl)-ethylamino]-3-methyl-butyl}-5-methyl-1H-[1,2,4]triazol-3-yl-benzoic acid;

1.6: 8-(2-{3-[3-(4-chloro-phenyl)-5-methyl-[1,2,4]triazol-1-yl]-1,1-dimethyl-propylamino}-1-hydroxy-ethyl)-6-hydroxy-4H-benzo[1,4]oxazin-3-one;

1.7: 8-(2-{3-[5-ethyl-3-(4-methoxy-phenyl)-[1,2,4]triazol-1-yl]-1,1-dimethyl-propylamino}-1-hydroxy-ethyl)-6-hydroxy-4H-benzo[1,4]oxazin-3-one;

1.8: 6-hydroxy-8-(1-hydroxy-2-{3-[3-(4-methoxy-phenyl)-5-methyl-[1,2,4]triazol-1-yl]-1,1-dimethyl-propylamino}-ethyl)-4H-benzo[1,4]oxazin-3-one;

1.9: 8-{2-[3-(3-benzo[1,3]dioxol-5-yl-5-methyl-[1,2,4]triazol-1-yl)-1,1-dimethyl-propylamino]-1-hydroxy-ethyl}-6-hydroxy-4H-benzo[1,4]oxazin-3-one;

1.10: 7-{2-[3-(3-benzo[1,3]dioxol-5-yl-5-methyl-[1,2,4]triazol-1-yl)-1,1-dimethyl-propylamino]-1-hydroxy-ethyl}-5-hydroxy-3H-benzooxazol-2-one and

1.11: 8-{2-[3-(3-benzo[1,3]dioxol-5-yl-5-methyl-[1,2,4]triazol-1-yl)-1,1-dimethyl-propylamino]-1-hydroxy-ethyl}-6-hydroxy-2,2-dimethyl-4H-benzo[1,4]oxazin-3-one,

optionally in the form of the pharmaceutically acceptable acid addition salts, hydrates or solvates thereof, at least one further active substance 2.

Particularly preferred are pharmaceutical combinations which contain, in addition to one or more, preferably one compound of general formula 1 selected from among the compounds

1.2: 8-{2-[1,1-dimethyl-3-(5-methyl-3-p-tolyl-[1,2,4]triazol-1yl)-propylamino]-1-hydroxy-ethyl}-6-hydroxy-4H-benzo[1,4]oxazin-3-one;

1.3: 8-(2-{1,1-dimethyl-3-[5-methyl-3-(4-trifluoromethyl-phenyl)-[1,2,4]triazol-1-yl]-propylamino}-1-hydroxy-ethyl)-6-hydroxy-4H-benzo[1,4]oxazin-3-one;

1.6: 8-(2-{3-[3-(4-chloro-phenyl)-5-methyl-[1,2,4]triazol-1-yl]-1,1-dimethyl-propylamino}-1-hydroxy-ethyl)-6-hydroxy-4H-benzo[1,4]oxazin-3-one;

1.7: 8-(2-{3-[5-ethyl-3-(4-methoxy-phenyl)-[1,2,4]triazol-1-yl]-1,1-dimethyl-propylamino}-1-hydroxy-ethyl)-6-hydroxy-4H-benzo[1,4]oxazin-3-one;

1.9: 8-{2-[3-(3-benzo[1,3]dioxol-5-yl-5-methyl-[1,2,4]triazol-1-yl)-1,1-dimethyl-propylamino]-1-hydroxy-ethyl}-6-hydroxy-4H-benzo[1,4]oxazin-3-one;

optionally in the form of the pharmaceutically acceptable acid addition salts, hydrates or solvates thereof, at least one further active substance 2.

Preferred pharmaceutical combinations contain in addition to one or more, preferably one compound of formula 1 as a further active substance one or more, preferably one anticholinergic 2a, optionally combined with pharmaceutically acceptable excipients.

In the pharmaceutical combinations according to the invention the anticholinergic 2a is preferably selected from among the tiotropium salts (2a.1), oxitropium salts (2a.2), flutropium salts (2a.3), ipratropium salts (2a.4), glycopyrronium salts (2a.5), trospium salts (2a.6) and the compounds of formulae 2a.7 to 2a.13.

In the above-mentioned salts 2a.1 to 2a.6 the cations tiotropium, oxitropium, flutropium, ipratropium, glycopyrronium and trospium are the pharmacologically active ingredients. Explicit reference to the above-mentioned cations is indicated by the terminology 2a.1′ to 2a.6′. Any reference to the above-mentioned salts 2a.1 to 2a.6 naturally also includes a reference to the corresponding cations tiotropium (2a.1′), oxitropium (2a.2′), flutropium (2a.3′), ipratropium (2a.4′), glycopyrronium (2a.5′), trospium (2a.6′).

By the salts 2a.1 to 2a.6 are meant according to the invention those compounds which contain in addition to the cations tiotropium (2a.1′), oxitropium (2a.2′), flutropium (2a.3′), ipratropium (2a.4′), glycopyrronium (2a.5′) and trospium (2a.6′) as counter-ion (anion) chloride, bromide, iodide, sulphate, phosphate, methanesulphonate, nitrate, maleate, acetate, citrate, fumarate, tartrate, oxalate, succinate, benzoate or p-toluenesulphonate, while chloride, bromide, iodide, sulphate, methanesulphonate or p-toluenesulphonate are preferred as counter-ions. Of all the salts the chlorides, bromides, iodides and methanesulphonates are particularly preferred. In the case of the trospium salts (2a.6) the chloride is particularly preferred. In the case of the other salts 2a.1 to 2a.5 the methanesulphonates and bromides are of particular importance. Of particular importance are medicament combinations which contain tiotropium salts (2a.1), oxitropium salts (2a.2) or ipratropium salts (2a.4), while the respective bromides are of particular importance according to the invention. The tiotropium bromide (2a.1) is of particular importance. The above-mentioned salts may optionally be present in the medicament combinations according to the invention in the form of the solvates or hydrates thereof, preferably in the form of their hydrates. In the case of tiotropium bromide the medicament combinations according to the invention preferably contain it in the form of the crystalline tiotropium bromide monohydrate which is known from WO 02/30928. If tiotropium bromide is used in anhydrous form in the medicament combinations according to the invention, preferably anhydrous crystalline tiotropium bromide is used, which is known from WO 03/000265.

Examples of novel preferred medicament combinations of preferred compounds of formula 1 with the above-mentioned anticholinergics 2a.1 to 2a.6 are combinations containing the compounds 1.2 and 2a.1; 1.2 and 2a.2; 1.2 and 2a.3; 1.2 and 2a.4; 1.2 and 2a.5; 1.2 and 2a.6; 1.3 and 2a.1; 1.3 and 2a.2; 1.3 and 2a.3; 1.3 and 2a.4; 1.3 and 2a.5; 1.3 and 2a.6; 1.6 and 2a.1; 1.6 and 2a.2; 1.6 and 2a.3; 1.6 and 2a.4; 1.6 and 2a.5; 1.6 and 2a.6; 1.7 and 2a.1; 1.7 and 2a.2; 1.7 and 2a.3; 1.7 and 2a.4; 1.7 and 2a.5; 1.7 and 2a.6; 1.9 and 2a.1; 1.9 and 2a.2; 1.9 and 2a.3; 1.9 and 2a.4; 1.9 and 2a.5; 1.9 and 2a.6; in each case optionally in the form of the racemates, enantiomers or diastereomers thereof and optionally in the form of the pharmacologically acceptable acid addition salts, solvates and/or hydrates thereof.

Of the combinations mentioned above, the preferred ones according to the invention are those which contain one of the compounds 2a.1, 2a.2 or 2a.4 as the compound of formula 2a, while those combinations which contain the compound 2a.1 are particularly important according to the invention.

Optionally the above-mentioned anticholinergics have chiral carbon centres. In this case the medicament combinations according to the invention may contain the anticholinergics in the form of their enantiomers, mixtures of enantiomers or racemates, while enantiomerically pure anticholinergics are preferably used.

In another preferred embodiment of the present invention the anticholinergics 2a contained in the medicament combinations according to the invention are selected from the salts of formula 2a.7

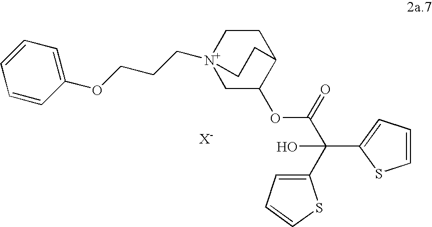

wherein
X⁻ denotes an anion with a single negative charge, preferably an anion selected from among the fluoride, chloride, bromide, iodide, sulphate, phosphate, methanesulphonate, nitrate, maleate, acetate, citrate, fumarate, tartrate, oxalate, succinate, benzoate and p-toluenesulphonate,
   optionally in the form of the racemates, enantiomers or hydrates thereof. Preferred medicament combinations contain salts of formula 2a.7, wherein
X⁻ denotes an anion with a single negative charge, preferably an anion selected from among the fluoride, chloride, bromide, methanesulphonate and p-toluenesulphonate, preferably bromide,
   optionally in the form of the racemates, enantiomers or hydrates thereof. Preferred medicament combinations contain salts of formula 2a.7, wherein
X⁻ denotes an anion with a single negative charge, preferably an anion selected from among the chloride, bromide and methanesulphonate, preferably bromide,
   optionally in the form of the racemates, enantiomers or hydrates thereof. Particularly preferred medicament combinations contain the compound of formula 2a.7 in the form of the bromide. Of particular importance are those medicament combinations which contain the enantiomers of formula 2a.7-ene

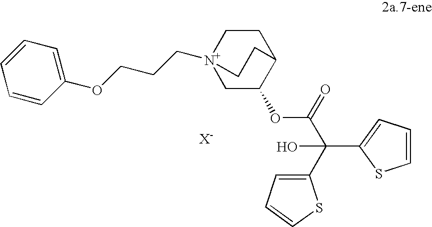

wherein X⁻ may have the meanings given above.

Examples of novel medicament combinations of preferred compounds of formula 1 with the above-mentioned anticholinergics 2a.7 are combinations containing the compounds 1.2 and 2a.7; 1.2 and 2a.7-ene; 1.3 and 2a.7; 1.3 and 2a.7-ene; 1.6 and 2a7; 16 and 2a.7-ene; 1.7 and 2a.7; 1.7 and 2a.7-ene; 1.9 and 2a.7; 1.9 and 2a.7-ene; in each case optionally in the form of the racemates, enantiomers or diastereomers thereof and optionally in the form of the pharmacologically acceptable acid addition salts, solvates and/or hydrates thereof.

In another preferred embodiment of the present invention the anticholinergics 2a contained in the medicament combinations according to the invention are selected from the salts of formula 2a.8

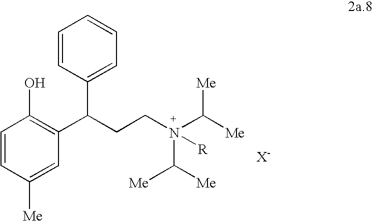

wherein R denotes either methyl (2a.8.1) or ethyl (2a.8.2) and wherein X⁻ may have the meanings given above. In an alternative embodiment the compound of formula 2a.8 is present in the form of the free base 2a.8-base

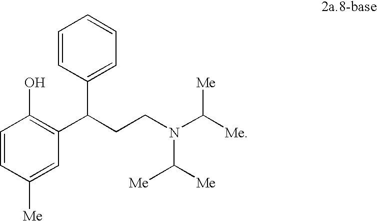

The medicament combinations according to the invention may contain the anticholinergic of formula 2a.8 (or 2a.8-base) in the form of the enantiomers, mixtures of enantiomers or racemates thereof. Preferably the anticholinergics of formula 2a.8 (or 2a.8-base) are present in the form of their R-enantiomers.

Examples of novel medicament combinations of preferred compounds of formula 1 with the above-mentioned anticholinergics 2a.8 are combinations containing the compounds 1.2 and 2a.8.1; 1.2 and 2a.8.2; 1.3 and 2a.8.1; 1.3 and 2a.8.2; 1.6 and 2a.8.1; 1.6 and 2a.8.2; 1.7 and 2a.8.1; 1.7 and 2a.8.2; 1.9 and 2a.8.1; 1.9 and 2a.8.2; in each case optionally in the form of the racemates, enantiomers or diastereomers thereof and optionally in the form of the pharmacologically acceptable acid addition salts, solvates and/or hydrates thereof.

In another preferred embodiment of the present invention the anticholinergics 2a contained in the medicament combinations according to the invention are selected from the compounds of formula 2a.9

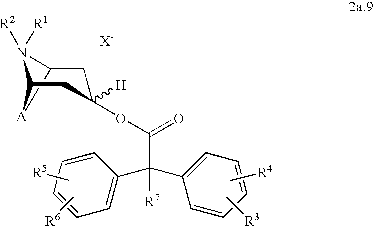

wherein
A denotes a double-bonded group selected from the groups

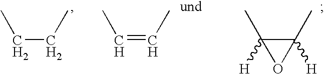

X⁻ denotes one of the above-mentioned anions with a single negative charge, preferably chloride, bromide or methanesulphonate,
R¹ and R² which may be identical or different denote a group selected from methyl, ethyl, n-propyl and iso-propyl, which may optionally be substituted by hydroxy or fluorine, preferably unsubstituted methyl;
R³, R⁴, R⁵ and R⁶, which may be identical or different, denote hydrogen, methyl, ethyl, methyloxy, ethyloxy, hydroxy, fluorine, chlorine, bromine, CN, CF₃ or NO₂;
R⁷ denotes hydrogen, methyl, ethyl, methyloxy, ethyloxy, —CH₂—F, —CH₂—CH₂—F, —O—CH₂F, —O—CH₂CH₂F, —CH₂OH, —CH₂CH₂OH, CF₃, —CH₂—OMe, —CH₂—CH₂—OMe, —CH₂—OEt, —CH₂—CH₂—OEt, —O—COMe, —O—COEt, —O—COCF₃, —O—COCF₃, fluorine, chlorine or bromine.

The compounds of formula 2a.9 are known in the art (WO 02/32899).

Within the scope of the medicament combinations according to the invention preferred compounds of formula 2a.9 are those wherein
X⁻ denotes bromide;
R¹ and R² which may be identical or different denote methyl or ethyl, preferably methyl;
R³, R⁴, R⁵ and R⁶, which may be identical or different, denote hydrogen, methyl, methyloxy, chlorine or fluorine;
R⁷ denotes hydrogen, methyl or fluorine.

Of particular importance are medicament combinations which contain those compounds of formula 2a.9, wherein
A denotes a double-bonded group selected from

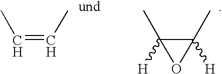

Of particular importance are those medicament combinations which contain in addition to a compound of formula 1 one of the following compounds of formula 2a.9:
tropenol 2,2-diphenylpropionate-methobromide (2a.9.1),
scopine 2,2-diphenylpropionate -methobromide (2a.9.2),
scopine 2-fluoro-2,2-diphenylacetate-methobromide (2a.9.3),
tropenol 2-fluoro-2,2-diphenylacetate-methobromide (2a.9.4);

The compounds of formula 2a.9 may optionally be present in the form of their enantiomers, mixtures of their enantiomers or racemates, as well as optionally in the form of the hydrates and/or solvates thereof.

Examples of novel medicament combinations of preferred compounds of formula 1 with the above-mentioned anticholinergics 2a.9 are combinations containing the compounds 1.2 and 2a.9.1; 1.2 and 2a.9.2; 1.2 and 2a.9.3; 1.2 and 2a.9.4; 1.3 and 2a.9.1; 1.3 and 2a.9.2; 1.3 and 2a.9.3; 1.3 and 2a.9.4; 1.6 and 2a.9.1; 1.6 and 2a.9.2; 1.6 and 2a.9.3; 1.6 and 2a.9.4; 1.7 and 2a.9.1; 1.7 and 2a.9.2; 1.7 and 2a.9.3; 1.7 and 2a.9.4; 1.9 and 2a.9.1; 1.9 and 2a.9.2; 1.9 and 2a.9.3; 1.9 and 2a.9.4; in each case optionally in the form of the racemates, enantiomers or diastereomers thereof and optionally in the form of the pharmacologically acceptable acid addition salts, solvates and/or hydrates thereof. Of the above-mentioned combinations the preferred ones according to the invention are those which contain as compound 2a.9 one of the compounds 2a.9.1 or 2a.9.2, while those combinations which contain the compound 2a.9.2 are particularly important according to the invention.

In another preferred embodiment of the present invention the anticholinergics 2a contained in the medicament combinations according to the invention are selected from the compounds of formula 2a.10

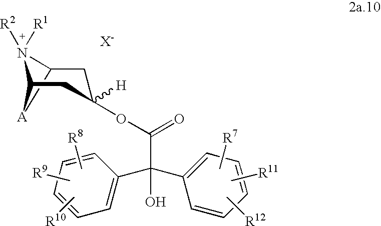

wherein
A, X, R¹ and R² may have the meanings given above and wherein R⁷, R⁸, R⁹, R¹⁰, R¹¹ and R¹², which may be identical or different, represent hydrogen, methyl, ethyl, methyloxy, ethyloxy, hydroxy, fluorine, chlorine, bromine, CN, CF₃ or NO₂, while at least one of the groups R⁷, R⁸, R⁹, R¹⁰, R¹¹ and R¹² may not be hydrogen.

The compounds of formula 2a.10 are known in the art (WO 02/32898).

Within the scope of the medicament combinations according to the invention preferred compounds of formula 2a.10 are those wherein
A denotes a double-bonded group selected from

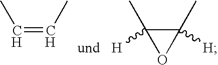

X⁻ denotes bromide;
R¹ and R² which may be identical or different, denote methyl or ethyl, preferably methyl;
R⁷ R⁸, R⁹, R¹⁰, R¹¹ and R¹², which may be identical or different, denote hydrogen, fluorine, chlorine or bromine, preferably fluorine, while at least one of the groups R⁷, R⁸, R⁹, R¹⁰, R¹¹ and R¹² may not be hydrogen.

Of particular importance are those medicament combinations which contain, in addition to a compound of formula 1, one of the following compounds of formula 2a.10:
tropenol 3,3′,4,4′-tetrafluorobenzilate-methobromide (2a.10.1),
scopine 3,3′,4,4′-tetrafluorobenzilate-methobromide (2a.10.2),
tropenol 4,4′-difluorobenzilate-methobromide (2a.10.3),
scopine 4,4′-difluorobenzilate-methobromide (2a.10.4),
tropenol 3,3′-difluorobenzilate-methobromide (2a.10.5),
scopine 3,3′-difluorobenzilate-methobromide (2a.10.6).

The compounds of formula 2a.10 may optionally be present in the form of the enantiomers, mixtures of enantiomers or racemates thereof, as well as optionally in the form of the hydrates and/or solvates thereof.

Examples of novel medicament combinations of preferred compounds of formula 1 with the above-mentioned anticholinergics 2a.10 are combinations containing the compounds 1.2 and 2a.10.1; 1.2 and 2a.10.2; 1.2 and 2a.10.3; 1.2 and 2a.10.4; 1.2 and 2a.10.5l 1.2 and 2a.10.6;1.3 and 2a.10.1; 1.3 and 2a.10.2; 1.3 and 2a.10.3; 1.3 and 2a.10.4; 1.3 and 2a.10.5; 1.3 and 2a.10.6; 1.6 and 2a.10.1; 1.6 and 2a.10.2; 1.6 and 2a.10.3; 1.6 and 2a.10.4; 1.6 and 2a.10.5; 1.6 and 2a.10.6; 1.7 and 2a.10.1; 1.7 and 2a.10.2; 1.7 and 2a.10.3; 1.7 and 2a.10.4; 1.7 and 2a.10.5; 1.7 and 2a.10.6; 1.9 and 2a.10.1; 1.9 and 2a.10.2; 1.9 and 2a.10.3; 1.9 and 2a.10.4; 1.9 and 2a.10.5; 1.9 and 2a.10.6; in each case optionally in the form of the racemates, enantiomers or diastereomers thereof and optionally in the form of the pharmacologically acceptable acid addition salts, solvates and/or hydrates thereof. Of the above-mentioned combinations the preferred ones according to the invention are those which contain as compound 2a.10 one of the compounds 2a.10.1, 2a.10.2, 2a.10.3 or 2a.10.4, while those combinations which contain the compounds 2a.10.1 or 2a.10.2 are particularly important according to the invention.

In another preferred embodiment of the present invention the anticholinergics 2a contained in the medicament combinations according to the invention are selected from the compounds of formula 2a.11

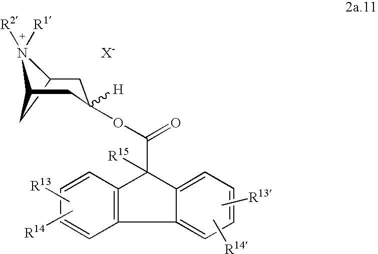

wherein
A and X⁻ may have the meanings given above and wherein
R¹⁵ denotes hydrogen, hydroxy, methyl, ethyl, —CF₃, CHF₂ or fluorine;
R^{1′} and R^{2′} which may be identical or different, denote C₁—C₅-alkyl, which may optionally be substituted by C₃—C₆-cycloalkyl, hydroxy or halogen, or
   R^{1′} and R^{2′} together denote a —C₃—C₅-alkylene bridge;
R¹³, R¹⁴, R^{13′} and R^{14′} which may be identical or different, represent hydrogen, —C₁—C₄-alkyl, —C₁—C₄-alkyloxy, hydroxy, —CF₃, —CHF₂, CN, NO₂ or halogen.

The compounds of formula 2a.11 are known in the art (WO 03/064419).

Within the scope of the medicament combinations according to the invention preferred compounds of formula 2a.11 are those wherein
A denotes a double-bonded group selected from

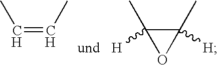

X⁻ denotes an anion selected from chloride, bromide and methanesulphonate, preferably bromide;
R¹⁵ denotes hydroxy, methyl or fluorine, preferably methyl or hydroxy;
R^{1′} and R^{2′} which may be identical or different denote methyl or ethyl, preferably methyl;
R¹³, R¹⁴, R^{13′} and R^{14′} which may be identical or different denote hydrogen, —CF₃, —CHF₂ or fluorine, preferably hydrogen or fluorine.

Within the scope of the medicament combinations according to the invention particularly preferred compounds of formula 2a.11 are those wherein
A denotes a double-bonded group selected from

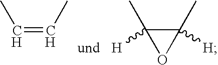

X⁻ denotes bromide;
R¹⁵ denotes hydroxy or methyl, preferably methyl;
R^{1′} and R^{2′} which may be identical or different denote methyl or ethyl, preferably methyl;
R¹³, R¹⁴, R^{13′} and R^{14′} which may be identical or different denote hydrogen or fluorine.

Of particular importance are those medicament combinations which contain, in addition to a compound of formula 1, one of the following compounds of formula 2a.11:
tropenol 9-hydroxy-fluorene-9-carboxylate methobromide (2a.11.1);
tropenol 9-fluoro-fluorene-9-carboxylate methobromide (2a.11.2);
scopine 9-hydroxy-fluorene-9-carboxylate methobromide (2a.11.3);
scopine 9-fluoro-fluorene-9-carboxylate methobromide (2a.11.4);
tropenol 9-methyl-fluorene-9-carboxylate methobromide (2a.11.5);
scopine 9-methyl-fluorene-9-carboxylate methobromide (2a.11.6);

The compounds of formula 2a.11 may optionally be present in the form of the enantiomers, mixtures of enantiomers or racemates thereof, as well as optionally in the form of the hydrates and/or solvates thereof.

Examples of novel medicament combinations of preferred compounds of formula 1 with the above-mentioned anticholinergics 2a.11 are combinations containing the compounds 1.2 and 2a.11.1; 1.2 and 2a.11.2; 1.2 and 2a.11.3; 1.2 and 2a.11.4; 1.2 and 2a.115; 1.2 and 2a.11.6;1.3 and 2a.11.1; 1.3 and 2a.11.2; 1.3 and 2a.11.3; 1.3 and 2a.11.4; 1.3 and 2a.11.5; 1.3 and 2a.11.6; 1.6 and 2a.11.1; 1.6 and 2a.11.2; 1.6 and 2a.11.3; 1.6 and 2a.11.4; 1.6 and 2a.11.5; 1.6 and 2a.11.6; 1.7 and 2a.11.1; 1.7 and 2a.11.2; 1.7 and 2a.11.3; 1.7 and 2a.11.4; 1.7 and 2a.11.5; 1.7 and 2a.11.6; 1.9 and 2a.11.1; 1.9 and 2a.11.2; 1.9 and 2a.11.3; 1.9 and 2a.11.4; 1.9 and 2a.11.5; 1.9 and 2a.11.6; in each case optionally in the form of the racemates, enantiomers or diastereomers thereof and optionally in the form of the pharmacologically acceptable acid addition salts, solvates and/or hydrates thereof. Of the above-mentioned combinations the preferred ones according to the invention are those which contain as compound 2a.11 one of the compounds 2a.11.2, 2a.11.4, 2a.11.5 or 2a.11.6, while those combinations which contain the compounds 2a.11.5 or 2a.11.6 are particularly important according to the invention.

In another preferred embodiment of the present invention the anticholinergics 2a contained in the medicament combinations according to the invention are selected from the compounds of formula 2a.12

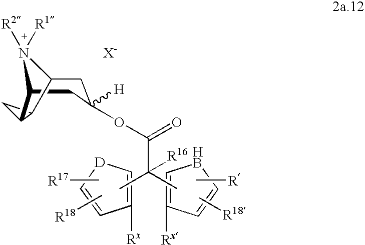

wherein X⁻ may have the meanings given above and wherein
D and B which may be identical or different, preferably identical, denote O, S, NH, CH₂, CH═CH or N(C₁—C₄-alkyl);
R¹⁶ denotes hydrogen, hydroxy, —C₁—C₄-alkyl, —C₁—C₄-alkyloxy, —C₁—C₄-alkylene-halogen, —O—C₁—C₄-alkylene-halogen, —C₁—C₄-alkylene-OH, —CF₃, CHF₂, —C₁—C₄-alkylene-C₁—C₄-alkyloxy, —O—COC₁—C₄-alkyl, —O—COC₁—C₄-alkylene-halogen, —C₁—C₄-alkylene-C₃—C₆-cycloalkyl, —O—COCF₃ or halogen;
R^{1″} and R^{2″} which may be identical or different, denote —C₁—C₅-alkyl, which may optionally be substituted by —C₃—C₆-cycloalkyl, hydroxy or halogen, or R^{1″} and R^{2″} together denote a —C₃—C₅-alkylene bridge;
R¹⁷, R¹⁸, R^{17′} and R^{18′}, which may be identical or different, denote hydrogen, —C₁—C₄-alkyl, —C₁—C₄-alkyloxy, hydroxy, —CF₃, —CHF₂, CN, NO₂ or halogen;
R^{x} and R^{x′} which may be identical or different denote hydrogen, —C₁—C₄-alkyl, —C₁—C₄-alkyloxy, hydroxy, —CF₃, —CHF₂, CN, NO₂ or halogen, or R^{x} and R^{x′} together denote a single bond or one of the double-bonded groups O, S, NH, CH₂, CH₂—CH₂, N(C₁—C₄-alkyl), CH(C₁—C₄-alkyl) and —C(C₁—C₄-alkyl)₂.

The compounds of formula 2a.12 are known in the art (WO 03/064418).

Within the scope of the medicament combinations according to the invention preferred compounds of formula 2a.12 are those wherein
X⁻ denotes chloride, bromide or methanesulphonate, preferably bromide;
D and B which may be identical or different, preferably identical, denote O, S, NH or CH═CH;
R¹⁶ denotes hydrogen, hydroxy, —C₁—C₄-alkyl, —C₁—C₄-alkyloxy, —CF₃, —CHF₂, fluorine, chlorine or bromine;
R^{1″} and R^{2″} which may be identical or different, denote C₁—C₄-alkyl, which may optionally be substituted by hydroxy, fluorine, chlorine or bromine, or R^{1″} and R^{2″} together denote a —C₃—C₄-alkylene bridge;
R¹⁷, R¹⁸, R^{17′} and R^{18′}, which may be identical or different, denote hydrogen, C₁—C₄-alkyl, C₁—C₄-alkyloxy, hydroxy, —CF₃, —CHF₂, CN, NO₂, fluorine, chlorine or bromine;
R^{x} and R^{x′} which may be identical or different denote hydrogen, C₁—C₄-alkyl, C₁—C₄-alkyloxy, hydroxy, —CF₃, —CHF₂, CN, NO₂, fluorine, chlorine or bromine, or R^{x} and R^{x′} together denote a single bond or a double-bonded group selected from O, S, NH— and CH₂.

Within the scope of the medicament combinations according to the invention particularly preferred compounds of formula 2a.12 are those wherein
X⁻ denotes chloride, bromide or methanesulphonate, preferably bromide;
D and B which may be identical or different, preferably identical, denote S or CH═CH;
R¹⁶ denotes hydrogen, hydroxy or methyl;
R^{1″} and R^{2″} which may be identical or different denote methyl or ethyl;
R¹⁷, R¹⁸, R^{17′} and R^{18′}, which may be identical or different, denote hydrogen, —CF₃ or fluorine, preferably hydrogen;
R^{x} and R^{x′} which may be identical or different denote hydrogen, —CF₃ or fluorine, preferably hydrogen, or R^{x} and R^{x′} together denote a single bond or —O.

Within the scope of the medicament combinations according to the invention particularly preferred compounds of formula 2a.12 are also those wherein
X⁻ denotes bromide;
D and B denote —CH═CH—;
R¹⁶ denotes hydrogen, hydroxy or methyl;
R^{1″} and R^{2″} denotes methyl;
R¹⁷, R¹⁸, R^{17′} and R^{18′}, which may be identical or different, denote hydrogen or fluorine, preferably hydrogen;
R^{x} and R^{x′} which may be identical or different denote hydrogen or fluorine, preferably hydrogen, or R^{x} and R^{x′} together denote a single bond or the group —O.

Of particular importance are those medicament combinations which contain in addition to a compound of formula 1 one of the following compounds of formula 2a.12:
cyclopropyltropine benzilate-methobromide (2a.12.1);
cyclopropyltropine 2,2-diphenylpropionate-methobromide (2a.12.2);
cyclopropyltropine 9-hydroxy-xanthene-9-carboxylate-methobromide (2a.12.3);
cyclopropyltropine 9-methyl-fluorene-9-carboxylate-methobromide (2a.12.4);
cyclopropyltropine 9-methyl-xanthene-9-carboxylate-methobromide (2a.12.5);
cyclopropyltropine 9-hydroxy-fluorene-9-carboxylate-methobromide (2a.12.6);
methyl cyclopropyltropine 4,4′-difluorobenzilate-methobromide (2a.12.7).

The compounds of formula 2a.12 may optionally be present in the form of the enantiomers, mixtures of enantiomers or racemates thereof, as well as optionally in the form of the hydrates and/or solvates thereof.

Examples of novel medicament combinations of preferred compounds of formula 1 with the above-mentioned anticholinergics 2a.12 are combinations containing the compounds 1.2 and 2a.12.1; 1.2 and 2a.12.2; 1.2 and 2a.12.3; 1.2 and 2a.12.4; 1.2 and 2a.12.5; 1.2 and 2a.12.6; 1.2 and 2a.12.7; 1.3 and 2a.12.1; 1.3 and 2a.12.2; 1.3 and 2a.12.3; 1.3 and 2a.12.4; 1.3 and 2a.12.5; 1.3 and 2a.12.6; 1.3 and 2a.12.7; 1.6 and 2a.12.1; 1.6 and 2a.12.2; 1.6 and 2a.12.3; 1.6 and 2a.12.4; 1.6 and 2a.12.5; 1.6 and 2a.12.6; 1.6 and 2a.12.7; 1.7 and 2a.12.1; 1.7 and 2a.12.2; 1.7 and 2a.12.3; 1.7 and 2a.12.4; 1.7 and 2a.12.5; 1.7 and 2a.12.6; 1.7 and 2a.12.7; 1.9 and 2a.12.1; 1.9 and 2a.12.2; 1.9 and 2a.12.3; 1.9 and 2a.12.4; 1.9 and 2a.12.5; 1.9 and 2a.12.6; 1.9 and 2a.12.7; in each case optionally in the form of the racemates, enantiomers or diastereomers thereof and optionally in the form of the pharmacologically acceptable acid addition salts, solvates and/or hydrates thereof. Of the above-mentioned combinations the preferred ones according to the invention are also those which contain as compound 2a.12 one of the compounds 2a.12.1, 2a.12.2, 2a.12.5 or 2a.12.7, while those combinations which contain the compounds 2a.12.1 or 2a.12.2 are of particular importance according to the invention.

In another preferred embodiment of the present invention the anticholinergics 2a contained in the medicament combinations according to the invention are selected from the compounds of formula 2a.13

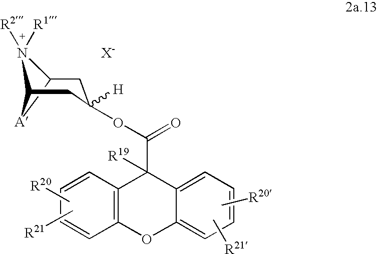

wherein X⁻ may have the meanings given above and wherein
A′ denotes a double-bonded group selected from

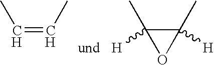

R¹⁹ denotes hydroxy, methyl, hydroxymethyl, ethyl, —CF₃, CHF₂ or fluorine;
R^{1′″} and R^{2′″} which may be identical or different, denote C₁—C₅-alkyl, which may optionally be substituted by C₃—C₆-cycloalkyl, hydroxy or halogen, or
   R^{1′″} and R^{2′″} together denote a —C₃—C₅-alkylene bridge;
R²⁰, R²¹, R^{20′} and R^{21′} which may be identical or different denote hydrogen, —C₁—C₄-alkyl, —C₁—C₄-alkyloxy, hydroxy, —CF₃, —CHF₂, CN, NO₂ or halogen.

The compounds of formula 2a.13 are known in the art (WO 03/064417).

Within the scope of the medicament combinations according to the invention preferred compounds of formula 2a.13 are those wherein
A′ denotes a double-bonded group selected from

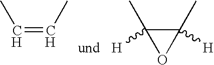

X⁻ denotes chloride, bromide or methanesulphonate, preferably bromide;
R¹⁹ denotes hydroxy or methyl;
R^{1′″} and R^{2′″} which may be identical or different denote methyl or ethyl, preferably methyl;
R²⁰, R²¹, R^{20′} and R^{21′} which may be identical or different denote hydrogen, —CF₃, —CHF₂ or fluorine, preferably hydrogen or fluorine.

Within the scope of the medicament combinations according to the invention particularly preferred compounds of formula 2a.13 are those wherein
A′ denotes a double-bonded group selected from

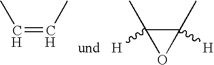

X⁻ denotes bromide;
R¹⁹ denotes hydroxy or methyl, preferably methyl;
R^{1′″} and R^{2′″} which may be identical or different denote methyl or ethyl, preferably methyl;
R³, R⁴, R^{3′} and R^{4′} which may be identical or different denote hydrogen or fluorine.

Of particular importance are those medicament combinations which contain in addition to a compound of formula 1 one of the following compounds of formula 2a.13:
tropenol 9-hydroxy-xanthene-9-carboxylate-methobromide (2a.13.1);
scopine 9-hydroxy-xanthene-9-carboxylate methobromide (2a.13.2);
tropenol 9-methyl-xanthene-9-carboxylate-methobromide (2a.13.3);
scopine 9-methyl-xanthene-9-carboxylate-methobromide (2a.13.4);
tropenol 9-ethyl-xanthene-9-carboxylate methobromide (2a.13.5);
tropenol 9-difluoromethyl-xanthene-9-carboxylate-methobromide (2a.13.6);
scopine 9-hydroxymethyl-xanthene-9-carboxylate-methobromide (2a.13.7).

The compounds of formula 2a.13 may optionally be present in the form of the enantiomers, mixtures of enantiomers or racemates thereof, as well as optionally in the form of the hydrates and/or solvates thereof.

Examples of novel medicament combinations of preferred compounds of formula 1 with the above-mentioned anticholinergics 2a.13 are combinations containing the compounds 1.2 and 2a.13.1; 1.2 and 2a.13.2; 1.2 and 2a.13.3; 1.2 and 2a.13.4; 1.2 and 2a.13.5; 1.2 and 2a.13.6; 1.2 and 2a.13.7; 1.3 and 2a.13.1; 1.3 and 2a.13.2; 1.3 and 2a.13.3; 1.3 and 2a.13.4; 1.3 and 2a.13.5; 1.3 and 2a.13.6; 1.3 and 2a.13.7; 1.6 and 2a.13.1; 1.6 and 2a.13.2; 1.6 and 2a.13.3; 1.6 and 2a.13.4; 1.6 and 2a.13.5; 1.6 and 2a.13.6; 1.6 and 2a.13.7; 1.7 and 2a.13.1; 1.7 and 2a.13.2; 1.7 and 2a.13.3; 1.7 and 2a.13.4; 1.7 and 2a.13.5; 1.7 and 2a.13.6; 1.7 and 2a.13.7; 1.9 and 2a.13.1; 1.9 and 2a.13.2; 1.9 and 2a.13.3; 1.9 and 2a.13.4; 1.9 and 2a.13.5; 1.9 and 2a.13.6; 1.9 and 2a.13.7; in each case optionally in the form of the racemates, enantiomers or diastereomers thereof and optionally in the form of the pharmacologically acceptable acid addition salts, solvates and/or hydrates thereof. Of the above-mentioned combinations the preferred ones according to the invention are also those which contain as compound 2a.11 one of the compounds 2a.13.2, 2a.13.3, 2a.13.4 or 2a.13.5, while those combinations which contain the compounds 2a.13.3 or 2a.13.4 are of particular importance according to the invention.

Within the scope of the present invention any reference to anticholinergics 1′ is to be taken as being a reference to the pharmacologically active cations of the salts in question. These cations are tiotropium (2a.1′), oxitropium (2a.2′), flutropium (2a.3′), ipratropium (2a.4′), glycopyrronium (2a.5′), trospium (2a.6′) and the cations listed below:

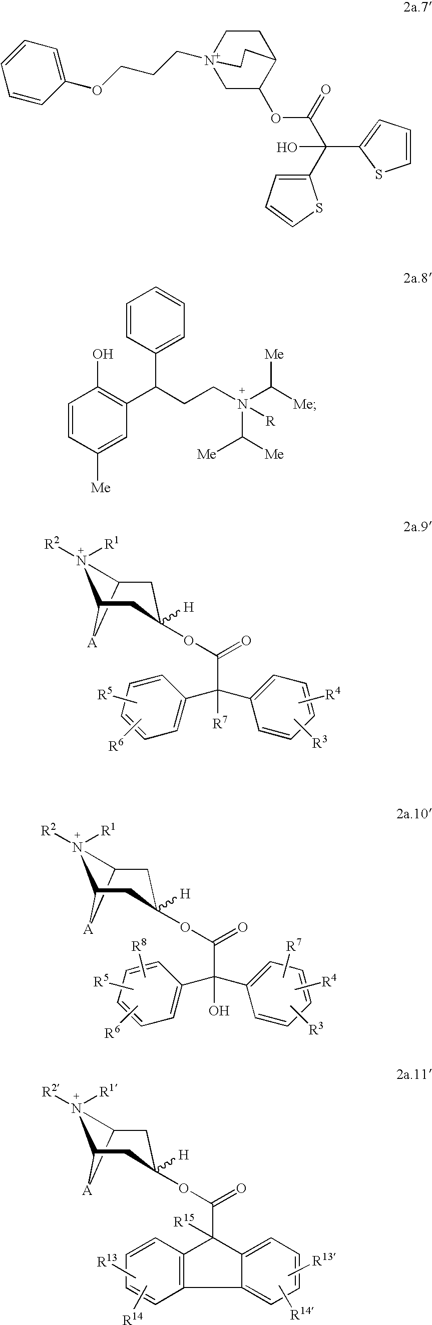

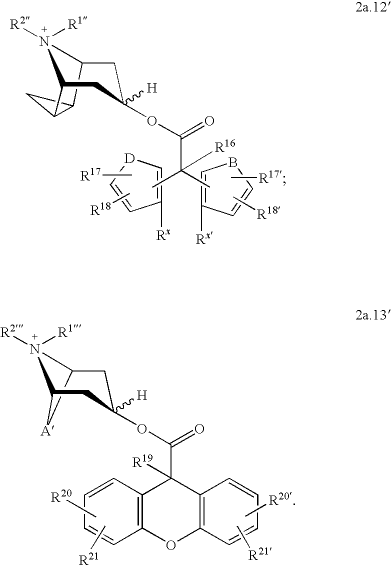

Other preferred medicament combinations according to the invention contain as a further active substance one or more, preferably one PDE IV inhibitor 2b in addition to one or more, preferably one compound of formula 1, optionally in combination with pharmaceutically acceptable excipients.

In medicament combinations of this kind the PDE IV inhibitor 2b is preferably selected from among enprofyllin, theophyllin, roflumilast, ariflo (cilomilast), CP-325,366, BY343, D-4396 (Sch-351591), AWD-12-281 (GW-842470), N-(3,5-dichloro-1-oxo-pyridin-4-yl)-4-difluoromethoxy-3-cyclopropylmethoxybenzamide, NCS-613, pumafentine, (−)ρ-[(4aR*,10bS*)-9-ethoxy-1,2,3,4,4a,10b-hexahydro-8-methoxy-2-methylbenzo[s][1,6]naphthyridin-6-yl]-N,N-diisopropylbenzamide, (R)-(+)-1-(4-bromobenzyl)-4-[(3-cyclopentyloxy)-4-methoxyphenyl]-2-pyrrolidone, 3-(cyclopentyloxy-4-methoxyphenyl)-1-(4-N′-[N-2-cyano-S-methyl-isothioureido]benzyl)-2-pyrrolidone, cis[4-cyano-4-(3-cyclopentyloxy-4-methoxyphenyl)cyclohexane-1-carboxylic acid], 2-carbomethoxy-4-cyano-4-(3-cyclopropyl methoxy-4-difluoromethoxyphenyl)cyclohexan-1-one, cis[4-cyano-4-(3-cyclopropyl methoxy-4-difluoromethoxyphenyl)cyclohexan-1-ol], (R)-(+)-ethyl[4-(3-cyclopentyloxy-4-methoxyphenyl)pyrrolidin-2-ylidene]acetate, (S)-(−)-ethyl[4-(3-cyclopentyloxy-4-methoxyphenyl)pyrrolidin-2-ylidene]acetate, CDP840, Bay-198004, D-4418, PD-168787, T-440, T-2585, arofyllin, atizoram, V-11294A, Cl-1018, CDC-801, CDC-3052, D-22888, YM-58997, Z-15370, 9-cyclopentyl-5,6-dihydro-7-ethyl-3-(2-thienyl)-9H-pyrazolo[3,4-c]-1,2,4-triazolo[4,3-a]pyridine and 9-cyclopentyl-5,6-dihydro-7-ethyl-3-(tert-butyl)-9H-pyrazolo[3.4-c]-1,2,4-triazolo[4,3-a]pyridine, optionally in the form of the racemates, enantiomers or diastereomers thereof and optionally in the form of the pharmacologically acceptable acid addition salts, solvates and/or hydrates thereof.

In particularly preferred medicament combinations the PDE IV inhibitor 2b is selected from among enprofyllin (2b.1), roflumilast (2b.2), ariflo (cilomilast) (2b.3), AWD-12-281 (GW-842470) (2b.4), N-(3,5-dichloro-1-oxo-pyridin-4-yl)-4-difluoromethoxy-3-cyclopropylmethoxybenzamide (2b.5), T-440 (2b.6), T-2585 (2b.7), arofyllin (2b.8), cis[4-cyano-4-(3-cyclopentyloxy-4-methoxyphenyl)cyclohexane-1-carboxylic acid] (2b.9), 2-carbomethoxy-4-cyano-4-(3-cyclopropyl methoxy-4-difluoromethoxyphenyl)cyclohexan-1-one (2b.10), cis[4-cyano-4-(3-cyclopropylmethoxy-4-difluoromethoxyphenyl)cyclohexan-1-ol] (2b.11), PD-168787 (2b.12), atizoram (2b.13), V-11294A (2b.14), Cl-1018 (2b.15), CDC-801 (2b.16), D-22888 (2b.17), YM-58997 (2b.18), Z-15370 (2b.19), 9-cyclopentyl-5,6-dihydro-7-ethyl-3-(2-thienyl)-9H-pyrazolo[3,4-c]-1,2,4-triazolo[4,3-a]pyridine (2b. 20) and 9-cyclopentyl-5,6-dihydro-7-ethyl-3-(tert-butyl)-9H-pyrazolo[3,4-c]-1,2,4-triazolo[4,3-a]pyridine (2b.21), optionally in the form of the racemates, enantiomers or diastereomers thereof and optionally in the form of the pharmacologically acceptable acid addition salts, solvates and/or hydrates thereof.

In particularly preferred medicament combinations the PDE IV inhibitor 2b is selected from among roflumilast (2b.2), ariflo (cilomilast) (2b.3), AWD-12-281 (GW-842470) (2b.4), arofyllin (2b.8), 2-carbomethoxy-4-cyano-4-(3-cyclopropylmethoxy-4-difluoromethoxyphenyl)cyclohexan-1-one (2b.10), cis[4-cyano-4-(3-cyclopropylmethoxy-4-difluoromethoxyphenyl)cyclohexan-1-ol] (2b.11), atizoram (2b.13), Z-15370 (2b.19), 9cyclopentyl-5,6-dihydro-7-ethyl-3-(2-thienyl)-9H-pyrazolo[3,4-c]-1,2,4-triazolo[4,3-a]pyridine (2b.20) and 9-cyclopentyl-5,6-dihydro-7-ethyl-3-(tert-butyl)-9H-pyrazolo[3,4-c]-1,2,4-triazolo[4,3-a]pyridine (2b.21), while roflumilast (2b.2), Z-15370 (2b.19) and AWD-12-281 (2b.4) are of particular importance, optionally in the form of the racemates, enantiomers or diastereomers thereof and optionally in the form of the pharmacologically acceptable acid addition salts, solvates and/or hydrates thereof.

By acid addition salts with pharmacologically acceptable acids, which the compounds 2b are optionally capable of forming, are meant for example salts selected from among the hydrochloride, hydrobromide, hydriodide, hydrosulphate, hydrophosphate, hydromethanesulphonate, hydronitrate, hydromaleate, hydroacetate, hydrobenzoate, hydrocitrate, hydrofumarate, hydrotartrate, hydroxalate, hydrosuccinate, hydrobenzoate and hydro-p-toluenesulphonate, preferably hydrochloride, hydrobromide, hydrosulphate, hydrophosphate, hydrofumarate and hydromethanesulphonate.

Examples of novel preferred medicament combinations of compounds of formula 1 with the above-mentioned PDE IV-inhibitors 2b are combinations containing the compounds 1.2 and 2b.1; 1.2 and 2b.2; 1.2 and 2b.3; 1.2 and 2b.4; 1.2 and 2b.5; 1.2 and 2b.6; 12 and 2b.7; 1.2 and 2b.8; 1.2 and 2b.9; 1.2 and 2b.10; 1.2 and 2b.11; 1.2 and 2b.12; 1.2 and 2b.13; 1.2 and 2b.14; 1.2 and 2b.15; 1.2 and 2b.16; 1.2 and 2b.17; 1.2 and 2b.18; 1.2 and 2b.19; 1.2 and 2b.20; 1.2 and 2b.21; 1.3 and 2b.1; 1.3 and 2b.2; 1.3 and 2b.3; 1.3 and 2b.4; 1.3 and 2b.5; 1.3 and 2b.6; 1.3 and 2b.7; 1.3 and 2b.8; 1.3 and 2b.9; 1.3 and 2b.10; 1.3 and 2b.11; 1.3 and 2b.12; 1.3 and 2b.13; 1.3 and 2b.14; 1.3 and 2b.15; 1.3 and 2b.16; 1.3 and 2b.17; 1.3 and 2b.18; 1.3 and 2b.19; 1.3 and 2b.20; 1.3 and 2b.21; 1.6 and 2b.1; 1.6 and 2b.2; 1.6 and 2b.3; 1.6 and 2b.4; 1.6 and 2b.5; 1.6 and 2b.6; 1.6 and 2b.7; 1.6 and 2b.8; 1.6 and 2b.9; 1.6 and 2b.10; 1.6 and 2b.11; 1.6 and 2b.6; 1.6 and 2b.13; 1.6 and 2b.14; 1.6 and 2b.15; 1.6 and 2b.16; 1.6 and 2b.17; 1.6 and 2b.18; 1.6 and 2b.19; 1.6 and 2b.20; 1.6 and 2b.21; 1.7 and 2b.1; 1.7 and 2b.2; 1.7 and 2b.3; 1.7 and 2b.4; 1.7 and 2b.5; 1.7 and 2b.6; 1.7 and 2b.7; 1.7 and 2b.8; 1.7 and 2b.9; 1.7 and 2b.10; 1.7 and 2b.11; 1.7 and 2b.12; 1.7 and 2b.13; 1.7 and 2b.14; 1.7 and 2b.15; 1.7 and 2b.16; 1.7 and 2b.17; 1.7 and 2b.18; 1.7 and 2b.19; 1.7 and 2b.20; 1.7 and 2b.21; 1.9 and 2b.1; 1.9 and 2b.2; 1.9 and 2b.3; 1.9 and 2b.4; 1.9 and 2b.5; 1.9 and 2b.6; 1.9 and 2b.7; 1.9 and 2b.8; 1.9 and 2b.9; 1.9 and 2b.10; 1.9 and 2b.11; 1.9 and 2b.12; 1.9 and 2b.13; 1.9 and 2b.14; 1.9 and 2b.15; 1.9 and 2b.16; 1.9 and 2b.17; 1.9 and 2b.18; 1.9 and 2b.19; 1.9 and 2b.20; 1.9 and 2b.21, in each case optionally in the form of the racemates, enantiomers or diastereomers thereof and optionally in the form of the pharmacologically acceptable acid addition salts, solvates and/or hydrates thereof. Of the above-mentioned combinations the preferred ones according to the invention are also those which contain as compound 2b one of the compounds 2b.2, 2b.3, 2b.4, 2b.8, 2b.10,2b.11, 2b.13, 2b.19, 2b.20 or 2b.21, while those combinations which contain one of the compounds 2b.2, 2b.4 or 2b.19 are particularly important according to the invention.

Other preferred medicament combinations according to the invention contain as a further active substance one or more, preferably one steroid 2c in addition to one or more, preferably one, compound of formula 1, optionally in combination with pharmaceutically acceptable excipients.

In medicament combinations of this kind the steroid 2c is preferably selected from among prednisolone (2c.1), prednisone (2c.2), butixocortpropionate (2c.3), RPR-106541 (2c.4), flunisolide (2c.5), beclomethasone (2c.6), triamcinolone (2c.7), budesonide (2c.8), fluticasone (2c.9), mometasone (2c.10), ciclesonide (2c.11), rofleponide (2c.12), ST-126 (2c.13), dexamethasone (2c.14), (S)-fluoromethyl 6α,9α-difluoro-17α-[(2-furanylcarbonyl)oxy]-11β-hydroxy-16α-methyl-3-oxo-androsta-1,4-diene-17β-carbothionate (2c.15), (S)-(2-oxo-tetrahydro-furan-3S-yl) 6α,9α-difluoro-11β-hydroxy-16α-methyl-3-oxo-17α-propionyloxy-androsta-1,4-diene-17β-carbothionate (2c.16) and etiprednol-dichloroacetate (BNP-166, 2c.17), optionally in the form of the racemates, enantiomers or diastereomers thereof and optionally in the form of the salts and derivatives thereof, the solvates and/or hydrates thereof.

In particularly preferred medicament combinations the steroid 2c is selected from among flunisolide (2c.5), beclomethasone (2c.6), triamcinolone (2c.7), budesonide (2c.8), fluticasone (2c.9), mometasone (2c.10), ciclesonide (2c.11), rofleponide (2c.12), ST-126 (2c.13), dexamethasone (2c.14), (S)-fluoromethyl 6α,9α-difluoro-17α-[(2-furanylcarbonyl)oxy]-11β-hydroxy-16α-methyl-3-oxo-androsta-1,4-diene-17β-carbothionate (2c.1 5), (S)-(2-oxo-tetrahydro-furan-3S-yl) 6α,9α-difluoro-11β-hydroxy-16α-methyl-3-oxo-17α-propionyloxy-androsta-1,4-diene-17β-carbothionate (2c.16) and etiprednol-dichloroacetate (2c.17), optionally in the form of the racemates, enantiomers or diastereomers thereof and optionally in the form of the salts and derivatives thereof, the solvates and/or hydrates thereof.

In particularly preferred medicament combinations the steroid 2c is selected from among budesonide (2c.8), fluticasone (2c.9), mometasone (2c.10), ciclesonide (2c.11), (S)-fluoromethyl 6α,9α-difluoro-17α-[(2-furanylcarbonyl)oxy]-11β-hydroxy-16α-methyl-3-oxo-androsta-1,4-diene-17β-carbothionate (2c.15) and etiprednol-dichloroacetate (2c.17), optionally in the form of the racemates, enantiomers or diastereomers thereof and optionally in the form of the salts and derivatives thereof, the solvates and/or hydrates thereof.

Any reference to steroids 2c includes a reference to any salts or derivatives, hydrates or solvates thereof which may exist. Examples of possible salts and derivatives of the steroids 2c may be: alkali metal salts, such as for example sodium or potassium salts, sulphobenzoates, phosphates, isonicotinates, acetates, propionates, dihydrogen phosphates, palmitates, pivalates or also furoates.

Examples of novel preferred medicament combinations of preferred compounds of formula 1 with the above-mentioned steroids 2c are combinations containing the compounds 1.2 and 2c.1; 1.2 and 2c.2; 1.2 and 2c.3; 1.2 and 2c.4; 1.2 and 2c.5; 1.2 and 2c.6; 1.2 and 2c.7; 1.2 and 2c.8; 1.2 and 2c.9; 1.2 and 2c.10; 1.2 and 2c.11; 1.2 and 2c.12; 1.2 and 2c.13; 1.2 and 2c.14; 1.2 and 2c.15; 1.2 and 2c.16; 1.2 and 2c.17; 1.3 and 2c.1; 1.3 and 2c.2; 1.3 and 2c.3; 1.3 and 2c.4; 1.3 and 2c.5; 1.3 and 2c.6; 1.3 and 2c.7; 1.3 and 2c.8; 1.3 and 2c.9; 1.3 and 2c.10; 1.3 and 2c.11; 1.3 and 2c.12; 1.3 and 2c.13; 1.3 and 2c.14; 1.3 and 2c.15; 1.3 and 2c.16; 1.3 and 2c.17; 1.6 and 2c.1; 1.6 and 2c.2; 1.6 and 2c.3; 1.6 and 2c.4; 1.6 and 2c.5; 1.6 and 2c.6; 1.6 and 2c.7; 1.6 and 2c.8; 1.6 and 2c.9; 1.6 and 2c.10; 1.6 and 2c.11; 1.6 and 2c.12; 1.6 and 2c.13; 1.6 and 2c.14; 1.6 and 2c.15; 1.6 and 2c.16; 1.6 and 2c.17; 1.7 and 2c.1; 1.7 and 2c.2; 1.7 and 2c.3; 1.7 and 2c.4; 1.7 and 2c.5; 1.7 and 2c.6; 1.7 and 2c.7; 1.7 and 2c.8; 1.7 and 2c.9; 1.7 and 2c.10; 1.7 and 2c.11; 1.7 and 2c.12; 1.7 and 2c.13; 1.7 and 2c.14; 1.7 and 2c.15; 1.7 and 2c.16; 1.7 and 2c.17; 1.9 and 2c.1; 1.9 and 2c.2; 1.9 and 2c.3; 1.9 and 2c.4; 1.9 and 2c.5; 1.9 and 2c.6; 1.9 and 2c.7; 1.9 and 2c.8; 1.9 and 2c.9; 1.9 and 2c.10; 1.9 and 2c.11; 1.9 and 2c.12; 1.9 and 2c.13; 1.9 and 2c.14; 1.9 and 2c.15; 1.9 and 2c.16; 1.9 and 2c.17; in each case optionally in the form of the racemates, enantiomers or diastereomers thereof and optionally in the form of the pharmacologically acceptable acid addition salts, solvates and/or hydrates thereof. Of the above-mentioned combinations the preferred ones according to the invention are also those which contain as compound 2c one of the compounds 2c.5, 2c.6, 2c.7, 2c.8, 2c.9, 2c.10, 2c.11, 2c.12, 2c.13, 2c.14, 2c.15, 2c.16 or 2c.17, while those combinations that contain one of the compounds 2c.8, 2c.9, 2c.10, 2c.11, 2c.15 or 2c.17 are of particular importance according to the invention.

Other preferred medicament combinations according to the invention contain, as an additional active substance, one or more, preferably one, LTD4 antagonist 2d in addition to one or more, preferably one compound of formula 1, optionally in combination with pharmaceutically acceptable excipients.

In such medicament combinations the LTD4 antagonist 2d is preferably selected from among montelukast (2d.1),1-(((R)-(3-(2-(6,7-difluoro-2-quinolinyl)ethenyl)phenyl)-3-(2-(2-hydroxy-2-propyl)phenyl)thio)methylcyclopropane-acetic acid (2d.2), 1-(((1(R)-3(3-(2-(2,3-dichlorothieno[3,2-b]pyridin-5-yl)-(E)-ethenyl)phenyl)-3-(2-(1-hydroxy-1-methylethyl)phenyl)propyl)thio)methyl)cyclopropanacetic acid (2d.3), pranlukast (2d.4), zafirlukast (2d.5), [2-[[2-(4-tert-butyl-2-thiazolyl)-5-benzofuranyl]oxymethyl]-phenyl]acetic acid (2d.6), MCC-847 (ZD-3523) (2d.7), MN-001 (2d.8), MEN-91507 (LM-1507) (2d.9), VUF-5078 (2d.10), VUF-K-8707 (2d.11) and L-733321 (2d.12), optionally in the form of the racemates, enantiomers or diastereomers thereof, optionally in the form of the pharmacologically acceptable acid addition salts thereof as well as optionally in the form of the salts and derivatives thereof, the solvates and/or hydrates thereof.

In preferred medicament combinations the LTD4 antagonist 2d is selected from the group comprising montelukast (2d.1), pranlukast (2d.4), zafirlukast (2d.5), MCC-847 (ZD-3523) (2d.7), MN-001 (2d.8), MEN-91507 (LM-1507) (2d.9), VUF-5078 (2d.10), VUF-K-8707 (2d.11) and L-733321 (2d.12), optionally in the form of the racemates, enantiomers or diastereomers thereof, optionally in the form of the pharmacologically acceptable acid addition salts thereof as well as optionally in the form of the salts and derivatives thereof, the solvates and/or hydrates thereof.

In particularly preferred medicament combinations the LTD4 antagonist 2d is selected from the group comprising montelukast (2d.1), pranlukast (2d.4), zafirlukast (2d.5), MCC-847 (ZD-3523) (2d.7), MN-001 (2d.8) and MEN-91507 (LM-1507) (2d.9), while montelukast (2d.1), pranlukast (2d.4) and zafirlukast (2d.5) are particularly preferred, optionally in the form of the racemates, enantiomers or diastereomers thereof, optionally in the form of the pharmacologically acceptable acid addition salts thereof as well as optionally in the form of the salts and derivatives thereof, the solvates and/or hydrates thereof.

By the acid addition salts with pharmacologically acceptable acids which the compounds 2d may possibly be capable of forming are meant for example salts selected from the group comprising the hydrochloride, hydrobromide, hydriodide, hydrosulphate, hydrophosphate, hydromethanesulphonate, hydronitrate, hydromaleate, hydroacetate, hydrobenzoate, hydrocitrate, hydrofumarate, hydrotartrate, hydroxalate, hydrosuccinate, hydrobenzoate and hydro-p-toluenesulphonate, preferably the hydrochloride, hydrobromide, hydrosulphate, hydrophosphate, hydrofumarate and hydromethanesulphonate.

Examples of possible salts and derivatives which the compounds 2d may possibly be capable of forming include for example: alkali metal salts, such as for example sodium or potassium salts, alkaline earth metal salts, sulphobenzoates, phosphates, isonicotinates, acetates, propionates, dihydrogen phosphates, palmitates, pivalates or furoates.

Examples of novel preferred medicament combinations of preferred compounds of formula 1 with the above-mentioned LTD4-antagonists 2d are combinations containing the compounds 1.2 and 2d.1; 1.2 and 2d.2; 1.2 and 2d.3; 1.2 and 2d.4; 1.2 and 2d.5; 1.2 and

2d.6; 1.2 and 2d.7; 1.2 and 2d.8; 1.2 and 2d.9; 1.2 and 2d.10; 1.2 and 2d.11; 1.2 and 2d.12; 1.3 and 2d.1; 1.3 and 2d.2; 1.3 and 2d.3; 1.3 and 2d.4; 1.3 and 2d.5; 1.3 and 2d.6; 1.3 and 2d.7; 1.3 and 2d.8; 1.3 and 2d.9; 1.3 and 2d.10; 1.3 and 2d.11; 1.3 and 2d.12; 1.6 and 2d.1; 1.6 and 2d.2; 1.6 and 2d.3; 1.6 and 2d.4; 1.6 and 2d.5; 1.6 and 2d.6; 1.6 and 2d.7; 1.6 and 2d.8; 1.6 and 2d.9; 1.6 and 2d.10; 1.6 and 2d.11; 1.6 and 2d.12; 17 and 2d.1; 1.7 and 2d.2; 1.7 and 2d.3; 1.7 and 2d.4; 1.7 and 2d.5; 1.7 and 2d.6; 1.7 and 2d.7; 1.7 and 2d.8; 1.7 and 2d.9; 1.7 and 2d.10; 1.7 and 2d.11; 1.7 and 2d.12; 1.9 and 2d.1; 1.9 and 2d.2; 1.9 and 2d.3; 1.9 and 2d.4; 1.9 and 2d.5; 1.9 and 2d.6; 1.9 and 2d.7; 1.9 and 2d.8; 1.9 and 2d.9; 1.9 and 2d.10; 1.9 and 2d.11; 1.9 and 2d.12; in each case optionally in the form of the racemates, enantiomers or diastereomers thereof and optionally in the form of the pharmacologically acceptable acid addition salts, solvates and/or hydrates thereof. Of the above-mentioned combinations the preferred ones according to the invention are also those which contain as compound 2d one of the compounds 2d.1, 2d.4, 2d.5, 2d.7, 2d.8, 2d.9, 2d.10, 2d.11 or2d.12, while those combinations that contain one of the compounds 2d.1, 2d.4, 2d.5, 2d.7, 2d.8 or 2d.9 are of particular importance according to the invention, while the combinations that contain one of the compounds 2d.1, 2d.4 or 2d.5 are of exceptional importance.

Other preferred medicament combinations according to the invention contain one or more, preferably one, EGFR-inhibitor 2e as an additional active substance in addition to one or more, preferably one compound of formula 1, optionally in combination with pharmaceutically acceptable excipients.

In such medicament combinations the EGFR-inhibitor 2e is selected for example from the group comprising 4-[(3-chloro-4-fluorophenyl)amino]-6-{[4-(morpholin-4-yl)-1-oxo-2-buten-1-yl]amino}-7-cyclopropylmethoxy-quinazoline, 4-[(3-chloro-4-fluorophenyl)amino]-6-{[4-(N,N-diethylamino)-1-oxo-2-buten-1-yl]amino}-7-cyclopropylmethoxy-quinazoline, 4-[(3-chloro-4-fluorophenyl)amino]-6-{[4-(N,N-dimethylamino)-1-oxo-2-buten-1-yl]amino}-7-cyclopropylmethoxy-quinazoline, 4-[(R)-(1-phenyl-ethyl)amino]-6-{[4-(morpholin-4-yl)-1-oxo-2-buten-1-yl]amino}-7-cyclopentyloxy-quinazoline, 4-[(3-chloro-4-fluoro-phenyl)amino]-6-{[4-((R)-6-methyl-2-oxo-morpholin-4-yl)-1-oxo-2-buten-1-yl]amino}-7-cyclopropylmethoxy-quinazoline, 4-[(3-chloro-4-fluoro-phenyl)amino]-6-{[4-((R)-6-methyl-2-oxo-morpholin-4-yl)-1-oxo-2-buten-1-yl]amino}-7-[(S)-(tetrahydrofuran-3-yl)oxy]-quinazoline, 4-[(3-chloro-4-fluoro-phenyl)amino]-6-{[4-((R)-2-methoxymethyl-6-oxo-morpholin-4-yl)-1-oxo-2-buten-1-yl]amino}-7-cyclopropylmethoxy-quinazoline, 4-[(3-chloro-4-fluoro-phenyl)amino]-6-[2-((S)-6-methyl-2-oxo-morpholin-4-yl)-ethoxy]-7-methoxy-quinazoline, 4-[(3-chloro-4-fluorophenyl)amino]-6-({4-[N-(2-methoxy-ethyl)-N-methyl-amino]-1-oxo-2-buten-1-yl}amino)-7-cyclopropylmethoxy-quinazoline, 4-[(3-chloro-4-fluorophenyl)amino]-6-{[4-(N,N-dimethylamino)-1-oxo-2-buten-1-yl]amino}-7-cyclopentyloxy-quinazoline, 4-[(R)-(1-phenyl-ethyl)amino]-6-{[4-(N,N-bis-(2-methoxy-ethyl)-amino)-1-oxo-2-buten-1-yl]amino}-7-cyclopropylmethoxy-quinazoline, 4-[(R)-(1-phenyl-ethyl)amino]-6-({4-[N-(2-methoxy-ethyl)-N-ethyl-amino]-1-oxo-2-buten-1-yl}amino)-7-cyclopropylmethoxy-quinazoline, 4-[(R)-(1-phenyl-ethyl)amino]-6-({4-[N-(2-methoxy-ethyl)-N-methyl-amino]-1-oxo-2-buten-1-yl}amino)-7-cyclopropylmethoxy-quinazoline, 4-[(R)-(1-phenyl-ethyl)amino]-6-({4-[N-(tetrahydropyran-4-yl)-N-methyl-amino]-1-oxo-2-buten-1-yl}amino)-7-cyclopropylmethoxy-quinazoline, 4-[(3-chloro-4-fluorophenyl)amino]-6-{[4-(N,N-dimethylamino)-1-oxo-2-buten-1-yl]amino}-7-((R)-tetrahydrofuran-3-yloxy)-quinazoline, 4-[(3-chloro-4-fluorophenyl)amino]-6-{[4-(N,N-dimethylamino)-1-oxo-2-buten-1-yl]amino}-7-((S)-tetrahydrofuran-3-yloxy)-quinazoline, 4-[(3-chloro-4-fluorophenyl)amino]-6-({4-[N-(2-methoxy-ethyl)-N-methyl-amino]-1-oxo-2-buten-1-yl}amino)-7-cyclopentyloxy-quinazoline, 4-[(3-chloro-4-fluorophenyl)amino]-6-{[4-(N-cyclopropyl-N-methyl-amino)-1-oxo-2-buten-1-yl]amino}-7-cyclopentyloxy-quinazoline, 4-[(3-chloro-4-fluorophenyl)amino]-6-{[4-(N,N-dimethylamino)-1-oxo-2-buten-1-yl]amino}-7-[(R)-(tetrahydrofuran-2-yl)methoxy]-quinazoline, 4-[(3-chloro-4-fluorophenyl)amino]-6-{[4-(N,N-dimethylamino)-1-oxo-2-buten-1-yl]amino}-7-[(S)-(tetrahydrofuran-2-yl)methoxy]-quinazoline, 4-[(3-ethynyl-phenyl)amino]-6,7-bis-(2-methoxy-ethoxy)-quinazoline, 4-[(3-chloro-4-fluorophenyl)amino]-7-[3-(morpholin-4-yl)-propyloxy]-6-[(vinylcarbonyl)amino]-quinazoline, 4-[(R)-(1-phenyl-ethyl)amino]-6-(4-hydroxy-phenyl)-7H-pyrrolo[2,3-d]pyrimidine, 3-cyano-4-[(3-chloro-4-fluorophenyl)amino]-6-{[4-(N,N-dimethylamino)-1-oxo-2-buten-1-yl]amino}-7-ethoxy-quinoline, 4-{[3-chloro-4-(3-fluoro-benzyloxy)-phenyl]amino}-6-(5-{[(2-methanesulphonyl-ethyl)amino]methyl}-furan-2-yl)quinazoline, 4-[(R)-( 1-phenyl-ethyl)amino]-6-{[4-((R)-6-methyl-2-oxo-morpholin-4-yl)-1-oxo-2-buten-1-yl]amino}-7-methoxy-quinazoline, 4-[(3-chloro-4-fluorophenyl)amino]-6-{[4-(morpholin-4-yl)-1-oxo-2-buten-1-yl]amino}-7-[(tetrahydrofuran-2-yl)methoxy]-quinazoline, 4-[(3-chloro-4-fluorophenyl)amino]-6-({4-[N,N-bis-(2-methoxy-ethyl)-amino]-1-oxo-2-buten-1-yl}amino)-7-[(tetrahydrofuran-2-yl)methoxy]-quinazoline, 4-[(3-ethynyl-phenyl)amino]-6-{[4-(5,5-dimethyl-2-oxo-morpholin-4-yl)-1-oxo-2-buten-1-yl]amino}-quinazoline, 4-[(3-chloro-4-fluoro-phenyl)amino]-6-[2-(2,2-dimethyl-6-oxo-morpholin-4-yl)-ethoxy]-7-methoxy-quinazoline, 4-[(3-chloro-4-fluoro-phenyl)amino]-6-[2-(2,2-dimethyl-6-oxo-morpholin-4-yl)-ethoxy]-7-[(R)-(tetrahydrofuran-2-yl)methoxy]-quinazoline, 4-[(3-chloro-4-fluoro-phenyl)amino]-7-[2-(2,2-dimethyl-6-oxo-morpholin-4-yl)-ethoxy]-6-[(S)-(tetrahydrofuran-2-yl)methoxy]-quinazoline, 4-[(3-chloro-4-fluoro-phenyl)amino]-6-{2-[4-(2-oxo-morpholin-4-yl)-piperidin-1-yl]-ethoxy}-7-methoxy-quinazoline, 4-[(3-chloro-4-fluoro-phenyl)amino]-6-[1-(tert-butyloxycarbonyl)-piperidin-4-yloxy]-7-methoxy-quinazoline, 4-[(3-chloro-4-fluoro-phenyl)amino]-6-(trans-4-amino-cyclohexan-1-yloxy)-7-methoxy-quinazoline, 4-[(3-chloro-4-fluoro-phenyl)amino]-6-(trans-4-methanesulphonylamino-cyclohexan-1-yloxy)-7-methoxy-quinazoline, 4-[(3-chloro-4-fluoro-phenyl)amino]-6-(tetrahydropyran-3-yloxy)-7-methoxy-quinazoline, 4-[(3-chloro-4-fluoro-phenyl)amino]-6-(1-methyl-piperidin-4-yloxy)-7-methoxy-quinazoline, 4-[(3-chloro-4-fluoro-phenyl)amino]-6-{1-[(morpholin-4-yl)carbonyl]-piperidin-4-yloxy}-7-methoxy-quinazoline, 4-[(3-chloro-4-fluoro-phenyl)amino]-6-{1-[(methoxymethyl)carbonyl]-piperidin-4-yloxy}-7-methoxy-quinazoline, 4-[(3-chloro-4-fluoro-phenyl)amino]-6-(piperidin-3-yloxy)-7-methoxy-quinazoline, 4-[(3-chloro-4-fluoro-phenyl)amino]-6-[1-(2-acetylamino-ethyl)-piperidin-4-yloxy]-7-methoxy-quinazoline, 4-[(3-chloro-4-fluoro-phenyl)amino]-6-(tetrahydropyran-4-yloxy)-7-ethoxy-quinazoline, 4-[(3-chloro-4-fluoro-phenyl)amino]-6-((S)-tetrahydrofuran-3-yloxy)-7-hydroxy-quinazoline, 4-[(3-chloro-4-fluoro-phenyl)amino]-6-(tetrahydropyran-4-yloxy)-7-(2-methoxy-ethoxy)-quinazoline, 4-[(3-chloro-4-fluoro-phenyl)amino]-6-{trans-4-[(dimethylamino)sulphonylamino]-cyclohexan-1-yloxy}-7-methoxy-quinazoline, 4-[(3-chloro-4-fluoro-phenyl)amino]-6-{trans-4-[(morpholin-4-yl)carbonylamino]-cyclohexan-1-yloxy}-7-methoxy-quinazoline, 4-[(3-chloro-4-fluoro-phenyl)amino]-6-{trans-4-[(morpholin-4-yl)sulphonylamino]-cyclohexan-1-yloxy}-7-methoxy-quinazoline, 4-[(3-chloro-4-fluoro-phenyl)amino]-6-(tetrahydropyran-4-yloxy)-7-(2-acetylamino-ethoxy)-quinazoline, 4-[(3-chloro-4-fluoro-phenyl)amino]-6-(tetrahydropyran-4-yloxy)-7-(2-methanesulphonylamino-ethoxy)-quinazoline, 4-[(3-chloro-4-fluoro-phenyl)amino]-6-{1-[(piperidin-1-yl)carbonyl]-piperidin-4-yloxy}-7-methoxy-quinazoline, 4-[(3-chloro-4-fluoro-phenyl)amino]-6-(1-aminocarbonylmethyl-piperidin-4-yloxy)-7-methoxy-quinazoline, 4-[(3-chloro-4-fluoro-phenyl)amino]-6-(cis-4-{N-[(tetrahydropyran-4-yl)carbonyl]-N-methyl-amino}-cyclohexan-1-yloxy)-7-methoxy-quinazoline, 4-[(3-chloro-4-fluoro-phenyl)amino]-6-(cis-4-{N-[(morpholin-4-yl)carbonyl]-N-methyl-amino}-cyclohexan-1-yloxy)-7-methoxy-quinazoline, 4-[(3-chloro-4-fluoro-phenyl)amino]-6-(cis-4-{N-[(morpholin-4-yl)sulphonyl]-N-methyl-amino}-cyclohexan-1-yloxy)-7-methoxy- quinazoline, 4-[(3-chloro-4-fluoro-phenyl)amino]-6-(trans-4-ethanesulphonylamino-cyclohexan-1-yloxy)-7-methoxy-quinazoline, 4-[(3-chloro-4-fluoro-phenyl)amino]-6-(1-methanesulphonyl-piperidin-4-yloxy)-7-ethoxy-quinazoline, 4-[(3-chloro-4-fluoro-phenyl)amino]-6-(1-methanesulphonyl-piperidin-4-yloxy)-7-(2-methoxy-ethoxy)-quinazoline, 4-[(3-chloro-4-fluoro-phenyl)amino]-6-[1-(2-methoxy-acetyl)-piperidin-4-yloxy]-7-(2-methoxy-ethoxy)-quinazoline, 4-[(3-chloro-4-fluoro-phenyl)amino]-6-(cis-4-acetylamino-cyclohexan-1-yloxy)-7-methoxy-quinazoline, 4-[(3-ethynyl-phenyl)amino]-6-[1-(tert.-butyloxycarbonyl)-piperidin-4-yloxy]-7-methoxy-quinazoline, 4-[(3-ethynyl-phenyl)amino]-6-(tetrahydropyran-4-yloxy]-7-methoxy-quinazoline, 4-[(3-chloro-4-fluoro-phenyl)amino]-6-(cis-4-{N-[(piperidin-1-yl)carbonyl]-N-methyl-amino}-cyclohexan-1-yloxy)-7-methoxy-quinazoline, 4-[(3-chloro-4-fluoro-phenyl)amino]-6-(cis-4-{N-[(4-methyl-piperazin-1-yl)carbonyl]-N-methyl-amino}-cyclohexan-1-yloxy)-7-methoxy-quinazoline, 4-[(3-chloro-4-fluoro-phenyl)amino]-6-{cis-4-[(morpholin-4-yl)carbonylamino]-cyclohexan-1-yloxy}-7-methoxy-quinazoline, 4-[(3-chloro-4-fluoro-phenyl)amino]-6-{1-[2-(2-oxopyrrolidin-1-yl)ethyl]-piperidin-4-yloxy}-7-methoxy-quinazoline, 4-[(3-chloro-4-fluoro-phenyl)amino]-6-{1-[(morpholin-4-yl)carbonyl]-piperidin-4-yloxy}-7-(2-methoxy-ethoxy)-quinazoline, 4-[(3-ethynyl-phenyl)amino]-6-(1-acetyl-piperidin-4-yloxy)-7-methoxy-quinazoline, 4-[(3-ethynyl-phenyl)amino]-6-(1-methyl-piperidin-4-yloxy)-7-methoxy-quinazoline, 4-[(3-ethynyl-phenyl)amino]-6-(1-methanesulphonyl-piperidin-4-yloxy)-7-methoxy-quinazoline, 4-[(3-chloro-4-fluoro-phenyl)amino]-6-(1-methyl-piperidin-4-yloxy)-7(2-methoxy-ethoxy)-quinazoline, 4-[(3-chloro-4-fluoro-phenyl)amino]-6-(1-isopropyloxycarbonyl-piperidin-4-yloxy)-7-methoxy-quinazoline, 4-[(3-chloro-4-fluoro-phenyl)amino]-6-(cis-4-methylamino-cyclohexan-1-yloxy)-7-methoxy-quinazoline, 4-[(3-chloro-4-fluoro-phenyl)amino]-6-{cis-4-[N-(2-methoxy-acetyl)-N-methyl-amino]-cyclohexan-1-yloxy}-7-methoxy-quinazoline, 4-[(3-ethynyl-phenyl)amino]-6-(piperidin-4-yloxy)-7-methoxy-quinazoline, 4-[(3-ethynyl-phenyl)amino]-6-[1-(2-methoxy-acetyl)-piperidin-4-yloxy]-7-methoxy-quinazoline, 4-[(3-ethynyl-phenyl)amino]-6-{1-[(morpholin-4-yl)carbonyl]-piperidin-4-yloxy}-7-methoxy-quinazoline, 4-[(3-chloro-4-fluoro-phenyl)amino]-6-{1-[(cis-2.6-dimethyl-morpholin-4-yl)carbonyl]-piperidin-4-yloxy}-7-methoxy-quinazoline, 4-[(3-chloro-4-fluoro-phenyl)amino]-6-{1-[(2-methyl-morpholin-4-yl)carbonyl]-piperidin-4-yloxy}-7-methoxy-quinazoline, 4-[(3-chloro-4-fluoro-phenyl)amino]-6-{1-[(S,S)-(2-oxa-5-aza-bicyclo[2,2,1]hept-5-yl)carbonyl]-piperidin-4-yloxy}-7-methoxy-quinazoline, 4-[(3-chloro-4-fluoro-phenyl)amino]-6-{1-[(N-methyl-N-2-methoxyethyl-amino)carbonyl]-piperidin-4-yloxy-}7-methoxy-quinazoline, 4-[(3-chloro-4-fluoro-phenyl)amino]-6-(1-ethyl-piperidin-4-yloxy)-7-methoxy-quinazoline, 4-[(3-chloro-4-fluoro-phenyl)amino]-6-{1-[(2-methoxyethyl)carbonyl]-piperidin-4-yloxy}-7-methoxy-quinazoline, 4-[(3-chloro-4-fluoro-phenyl)amino]-6-{1-[(3-methoxypropyl-amino)-carbonyl]-piperidin-4-yloxy}-7-methoxy-quinazoline, 4-[(3-chloro-4-fluoro-phenyl)amino]-6-[cis-4-(N-methanesulphonyl-N-methyl-amino)-cyclohexan-1-yloxy]-7-methoxy-quinazoline, 4-[(3-chloro-4-fluoro-phenyl)amino]-6-[cis-4-(N-acetyl-N-methyl-amino)-cyclohexan-1-yloxy]-7-methoxy-quinazoline, 4-[(3-chloro-4-fluoro-phenyl)amino]-6-(trans-4-methylamino-cyclohexan-1-yloxy)-7-methoxy-quinazoline, 4-[(3-chloro-4-fluoro-phenyl)amino]-6-[trans-4-(N-methanesulphonyl-N-methyl-amino)-cyclohexan-1-yloxy]-7-methoxy-quinazoline, 4-[(3-chloro-4-fluoro-phenyl)amino]-6-(trans-4-dimethylamino-cyclohexan-1-yloxy)-7-methoxy-quinazoline, 4-[(3-chloro-4-fluoro-phenyl)amino]-6-(trans-4-{N-[(morpholin-4-yl)carbonyl]-N-methyl-amino}-cyclohexan-1-yloxy)-7-methoxy-quinazoline, 4-[(3-chloro-4-fluoro-phenyl)amino]-6-[2-(2,2-dimethyl-6-oxo-morpholin-4-yl)-ethoxy]-7-[(S)-(tetrahydrofuran-2-yl)methoxy]-quinazoline, 4-[(3-chloro-4-fluoro-phenyl)amino]-6-(1-methanesulphonyl-piperidin-4-yloxy)-7-methoxy-quinazoline, 4-[(3-chloro-4-fluoro-phenyl)amino]-6-(1-cyano-piperidin-4-yloxy)-7-methoxy-quinazoline, cetuximab, trastuzumab, ABX-EGF and Mab ICR-62, optionally in the form of the racemates, enantiomers or diastereomers thereof, optionally in the form of the pharmacologically acceptable acid addition salts thereof, the solvates and/or hydrates thereof.

In such medicament combinations the EGFR-inhibitor 2e is preferably selected from among 4-[(3-chloro-4-fluorophenyl)amino]-6-{[4-(morpholin-4-yl)-1-oxo-2-buten-1-yl]-amino}-7-cyclopropylmethoxy-quinazoline, 4-[(3-chloro-4-fluorophenyl)amino]-6-{[4-(N,N-diethylamino)-1-oxo-2-buten-1-yl]amino}-7-cyclopropylmethoxy-quinazoline, 4-[(3-chloro-4-fluorophenyl)amino]-6-{[4-(N,N-dimethylamino)-1-oxo-2-buten-1-yl]amino}-7-cyclopropylmethoxy-quinazoline, 4-[(R)-(1-phenyl-ethyl)amino]-6-{[4-(morpholin-4-yl)-1-oxo-2-buten-1-yl]amino}-7-cyclopentyloxy-quinazoline, 4-[(3-chloro-4-fluoro-phenyl)amino]-6-{[4-((R)-6-methyl-2-oxo-morpholin-4-yl)-1-oxo-2-buten-1-yl]amino}-7-cyclopropylmethoxy-quinazoline, 4-[(3-chloro-4-fluoro-phenyl)amino]-6-{[4-((R)-6-methyl-2-oxo-morpholin-4-yl)-1-oxo-2-buten-1-yl]amino}-7-[(S)-(tetrahydrofuran-3-yl)oxy]-quinazoline, 4-[(3-chloro-4-fluoro-phenyl)amino]-6-{[4-((R)-2-methoxymethyl-6-oxo-morpholin-4-yl)-1-oxo-2-buten-1-yl]amino}-7-cyclopropylmethoxy-quinazoline, 4-[(3-chloro-4-fluoro-phenyl)amino]-6-[2-((S)-6-methyl-2-oxo-morpholin-4-yl)-ethoxy]-7-methoxy-quinazoline, 4-[(3-chloro-4-fluorophenyl)amino]-6-({4-[N-(2-methoxy-ethyl)-N-methyl-amino]-1-oxo-2-buten-1-yl}amino)-7-cyclopropylmethoxy-quinazoline, 4-[(3-chloro-4-fluorophenyl)amino]-6-{[4-(N,N-dimethylamino)-1-oxo-2-buten-1-yl]amino}-7-cyclopentyloxy-quinazoline, 4-[(R)-(1-phenyl-ethyl)amino]-6-{[4-(N,N-bis-(2-methoxy-ethyl)-amino)-1-oxo-2-buten-1-yl]amino}-7-cyclopropylmethoxy-quinazoline, 4-[(R)-(1-phenyl-ethyl)amino]-6-({4-[N-(2-methoxy-ethyl)-N-ethyl-amino]-1-oxo-2-buten-1-yl}amino)-7-cyclopropylmethoxy-quinazoline, 4-[(R)-(1-phenyl-ethyl)amino]-6-({4-[N-(2-methoxy-ethyl)-N-methyl-amino]-1-oxo-2-buten-1-yl}amino)-7-cyclopropylmethoxy-quinazoline, 4-[(R)-(1-phenyl-ethyl)amino]-6-({4-[N-(tetrahydropyran-4-yl)-N-methyl-amino]-1-oxo-2-buten-1-yl}amino)-7-cyclopropylmethoxy-quinazoline, 4-[(3-chloro-4-fluorophenyl)amino]-6-{[4-(N,N-dimethylamino)-1-oxo-2-buten-1-yl]amino}-7-((R)-tetrahydrofuran-3-yloxy)-quinazoline, 4-[(3-chloro-4-fluorophenyl)amino]-6-{[4-(N,N-dimethylamino)-1-oxo-2-buten-1-yl]amino}-7-((S)-tetrahydrofuran-3-yloxy)-quinazoline, 4-[(3-chloro-4-fluorophenyl)amino]-6-({4-[N-(2-methoxy-ethyl)-N-methyl-amino]-1-oxo-2-buten-1-yl}amino)-7-cyclopentyloxy-quinazoline, 4-[(3-chloro-4-fluorophenyl)amino]-6-{[4-(N-cyclopropyl-N-methyl-amino)-1-oxo-2-buten-1-yl]amino}-7-cyclopentyloxy-quinazoline, 4-[(3-chloro-4-fluorophenyl)amino]-6-{[4-(N,N-dimethylamino)-1-oxo-2-buten-1-yl]amino}-[(R)-(tetrahydrofuran-2-yl)methoxy]-quinazoline, 4-[(3-chloro-4-fluorophenyl)amino]-6-{[4-(N,N-dimethylamino)-1-oxo-2-buten-1-yl]amino}-7-[(S)-(tetrahydrofuran-2-yl)methoxy]-quinazoline, 4-[(3-ethynyl-phenyl)amino]-6,7-bis-(2-methoxy-ethoxy)-quinazoline, 4-[(3-chloro-4-fluorophenyl)amino]-7-[3-(morpholin-4-yl)-propyloxy]-6-[(vinylcarbonyl)amino]-quinazoline, 4-[(R)-(1-phenyl-ethyl)amino]-6-(4-hydroxy-phenyl)-7H-pyrrolo[2,3-d]pyrimidine, 3-cyano-4-[(3-chloro-4-fluorophenyl)amino]-6-{[4-(N,N-dimethylamino)-1-oxo-2-buten-1-yl]amino}-7-ethoxy-quinoline, 4-{[3-chloro-4-(3-fluoro-benzyloxy)-phenyl]amino}-6-(5-{[(2-methanesulphonyl-ethyl)amino]methyl}-furan-2-yl)quinazoline, 4-[(R)-( 1-phenyl-ethyl)amino]-6-{[4-((R)-6-methyl-2-oxo-morpholin-4-yl)-1-oxo-2-buten-1-yl]amino}-7-methoxy-quinazoline, 4-[(3-chloro-4-fluorophenyl)amino]-6-{[4-(morpholin-4-yl)-1-oxo-2-buten-1-yl]amino}-7-[(tetrahydrofuran-2-yl)methoxy]-quinazoline, 4-[(3-chloro-4-fluorophenyl)amino]-6-({4-[N,N-bis-(2-methoxy-ethyl)-amino]-1-oxo-2-buten-1-yl}amino)-7-[(tetrahydrofuran-2-yl)methoxy]-quinazoline, 4-[(3-ethynyl-phenyl)amino]-6-{[4-(5,5-dimethyl-2-oxo-morpholin-4-yl)-1-oxo-2-buten-1-yl]amino}-quinazoline, 4-[(3-chloro-4-fluoro-phenyl)amino]-6-[2-(2,2-dimethyl-6-oxo-morpholin-4-yl)-ethoxy]-7-methoxy-quinazoline, 4-[(3-chloro-4-fluoro-phenyl)amino]-6-[2-(2,2-dimethyl-6-oxo-morpholin-4-yl)-ethoxy]-7-[(R)-(tetrahydrofuran-2-yl)methoxy]-quinazoline, 4-[(3-chloro-4-fluoro-phenyl)amino]-7-[2-(2,2-dimethyl-6-oxo-morpholin-4-yl)-ethoxy]-6-[(S)-(tetrahydrofuran-2-yl)methoxy]-quinazoline, 4-[(3-chloro-4-fluoro-phenyl)amino]-6-{2-[4-(2-oxo-morpholin-4-yl)-piperidin-1-yl]-ethoxy}-7-methoxy-quinazoline, 4-[(3-chloro-4-fluoro-phenyl)amino]-6-[1-(tert-butyloxycarbonyl)-piperidin-4-yloxy]-7-methoxy-quinazoline, 4-[(3-chloro-4-fluoro-phenyl)amino]-6-(trans-4-amino-cyclohexan-1-yloxy)-7-methoxy-quinazoline, 4-[(3-chloro-4-fluoro-phenyl)amino]-6-(trans-4-methanesulphonylamino-cyclohexan-1-yloxy)-7-methoxy-quinazoline, 4-[(3-chloro-4-fluoro-phenyl)amino]-6-(tetrahydropyran-3-yloxy)-7-methoxy-quinazoline, 4-[(3-chloro-4-fluoro-phenyl)amino]-6-(1-methyl-piperidin-4-yloxy)-7-methoxy-quinazoline, 4-[(3-chloro-4-fluoro-phenyl)amino]-6-{1-[(morpholin-4-yl)carbonyl]-piperidin-4-yloxy}-7-methoxy-quinazoline, 4-[(3-chloro-4-fluoro-phenyl)amino]-6-{1-[(methoxymethyl)carbonyl]-piperidin-4-yloxy}-7-methoxy-quinazoline, 4-[(3-chloro-4-fluoro-phenyl)amino]-6-(piperidin-3-yloxy)-7-methoxy-quinazoline, 4-[(3-chloro-4-fluoro-phenyl)amino]-6-[1-(2-acetylamino-ethyl)-piperidin-4-yloxy]-7-methoxy-quinazoline, 4-[(3-chloro-4-fluoro-phenyl)amino]-6-(tetrahydropyran-4-yloxy)-7-ethoxy-quinazoline, 4-[(3-chloro-4-fluoro-phenyl)amino]-6-((S)-tetrahydrofuran-3-yloxy)-7-hydroxy-quinazoline, 4-[(3-chloro-4-fluoro-phenyl)amino]-6-(tetrahydropyran-4-yloxy)-7-(2-methoxy-ethoxy)-quinazoline, 4-[(3-chloro-4-fluoro-phenyl)amino]-6-{trans-4-[(dimethylamino)sulphonylamino]-cyclohexan-1-yloxy}-7-methoxy-quinazoline, 4-[(3-chloro-4-fluoro-phenyl)amino]-6-{trans-4-[(morpholin-4-yl)carbonylamino]-cyclohexan-1-yloxy}-7-methoxy-quinazoline, 4-[(3-chloro-4-fluoro-phenyl)amino]-6-{trans-4-[(morpholin-4-yl)sulphonylamino]-cyclohexan-1-yloxy}-7-methoxy-quinazoline, 4-[(3-chloro-4-fluoro-phenyl)amino]-6-(tetrahydropyran-4-yloxy)-7-(2-acetylamino-ethoxy)-quinazoline, 4-[(3-chloro-4-fluoro-phenyl)amino]-6-(tetrahydropyran-4-yloxy)-7-(2-methanesulphonylamino-ethoxy)-quinazoline, 4-[(3-chloro-4-fluoro-phenyl)amino]-6-{1-[(piperidin-1-yl)carbonyl]-piperidin-4-yloxy}-7-methoxy-quinazoline, 4-[(3-chloro-4-fluoro-phenyl)amino]-6-(1-aminocarbonylmethyl-piperidin-4-yloxy)-7-methoxy-quinazoline, 4-[(3-chloro-4-fluoro-phenyl)amino]-6-(cis-4-1N-[(tetrahydropyran-4-yl)carbonyl]-N-methyl-amino}-cyclohexan-1-yloxy)-7-methoxy-quinazoline, 4-[(3-chloro-4-fluoro-phenyl)amino]-6-(cis-4-{N-[(morpholin-4-yl)carbonyl]-N-methyl-amino}-cyclohexan-1-yloxy)-7-methoxy-quinazoline, 4-[(3-chloro-4-fluoro-phenyl)amino]-6-(cis-4-{N-[(morpholin-4-yl)sulphonyl]-N-methyl-amino}-cyclohexan-1-yloxy)-7-methoxy-quinazoline, 4-[(3-chloro-4-fluoro-phenyl)amino]-6-(trans-4-ethanesulphonylamino-cyclohexan-1-yloxy)-7-methoxy-quinazoline, 4-[(3-chloro-4-fluoro-phenyl)amino]-6-(1-methanesulphonyl-piperidin-4-yloxy)-7-ethoxy-quinazoline, 4-[(3-chloro-4-fluoro-phenyl)amino]-6-(1-methanesulphonyl-piperidin-4-yloxy)-7-(2-methoxy-ethoxy)-quinazoline, 4-[(3-chloro-4-fluoro-phenyl)amino]-6-[1-(2-methoxy-acetyl)-piperidin-4-yloxy]-7-(2-methoxy-ethoxy)-quinazoline, 4-[(3-chloro-4-fluoro-phenyl)amino]-6-(cis-4-acetylamino-cyclohexan-1-yloxy)-7-methoxy-quinazoline, 4-[(3-ethynyl-phenyl)amino]-6-[1-(tert.-butyloxycarbonyl)-piperidin-4-yloxy]-7-methoxy-quinazoline, 4-[(3-ethynyl-phenyl)amino]-6-(tetrahydropyran-4-yloxy]-7-methoxy-quinazoline, 4-[(3-chloro-4-fluoro-phenyl)amino]-6-(cis-4-{N-[(piperidin-1-yl)carbonyl]-N-methyl-amino}-cyclohexan-1-yloxy)-7-methoxy-quinazoline, 4-[(3-chloro-4-fluoro-phenyl)amino]-6-(cis-4-{N-[(4-methyl-piperazin-1-yl)carbonyl]-N-methyl-amino}-cyclohexan-1-yloxy)-7-methoxy-quinazoline, 4-[(3-chloro-4-fluoro-phenyl)amino]-6-{cis-4-[(morpholin-4-yl)carbonylamino]-cyclohexan-1-yloxy}-7-methoxy-quinazoline, 4-[(3-chloro-4-fluoro-phenyl)amino]-6-{1-[2-(2-oxopyrrolidin-1-yl)ethyl]-piperidin-4-yloxy}-7-quinazoline, 4-[(3-chloro-4-fluoro-phenyl)amino]-6-{1-[(morpholin-4-yl)carbonyl]-piperidin-4-yloxy}-7-(2-methoxy-ethoxy)-quinazoline, 4-[(3-ethynyl-phenyl)amino]-6-( 1-acetyl-piperidin-4-yloxy)-7-methoxy-quinazoline, 4-[(3-ethynyl-phenyl)amino]-6-(1-methyl-piperidin-4-yloxy)-7-methoxy-quinazoline, 4-[(3-ethynyl-phenyl)amino]-6-(1-methanesulphonyl-piperidin-4-yloxy)-7-methoxy-quinazoline, 4-[(3-chloro-4-fluoro-phenyl)amino]-6-(1-methyl-piperidin-4-yloxy)-7(2-methoxy-ethoxy)-quinazoline, 4-[(3-chloro-4-fluoro-phenyl)amino]-6-( 1-isopropyloxycarbonyl-piperidin-4-yloxy)-7-methoxy-quinazoline, 4-[(3-chloro-4-fluoro-phenyl)amino]-6-(cis-4-methylamino-cyclohexan-1-yloxy)-7-methoxy-quinazoline, 4-[(3-chloro-4-fluoro-phenyl)amino]-6-{cis-4-[N-(2-methoxy-acetyl)-N-methyl-amino]-cyclohexan-1-yloxy}-7-methoxy-quinazoline, 4-[(3-ethynyl-phenyl)amino]-6-(piperidin-4-yloxy)-7-methoxy-quinazoline, 4-[(3-ethynyl-phenyl)amino]-6-[1-(2-methoxy-acetyl)-piperidin-4-yloxy]-7-methoxy-quinazoline, 4-[(3-ethynyl-phenyl)amino]-6-{1-[(morpholin-4-yl)carbonyl]-piperidin-4-yloxy}-7-methoxy-quinazoline, 4-[(3-chloro-4-fluoro-phenyl)amino]-6-{1-[(cis-2.6-dimethyl-morpholin-4-yl)carbonyl]-piperidin-4-yloxy}-7-methoxy-quinazoline, 4-[(3-chloro-4-fluoro-phenyl)amino]-6-{1-[(2-methyl-morpholin-4-yl)carbonyl]-piperidin-4-yloxy}-7-methoxy-quinazoline, 4-[(3-chloro-4-fluoro-phenyl)amino]-6-{1-[(S,S)-(2-oxa-5-aza-bicyclo[2,2, 1 ]hept-5-yl)carbonyl]-piperidin-4-yloxy}-7-methoxy-quinazoline, 4-[(3-chloro-4-fluoro-phenyl)amino]-6-{1-[(N-methyl-N-2-methoxyethyl-amino)carbonyl]-piperidin-4-yloxy}-7-methoxy-quinazoline, 4-[(3-chloro-4-fluoro-phenyl)amino]-6-(1-ethyl-piperidin-4-yloxy)-7-methoxy-quinazoline, 4-[(3-chloro-4-fluoro-phenyl)amino]-6-{1-[(2-methoxyethyl)carbonyl]-piperidin-4-yloxy}-7-methoxy-quinazoline, 4-[(3-chloro-4-fluoro-phenyl)amino]-6-{1-[(3-methoxypropyl-amino)-carbonyl]-piperidin-4-yloxy}-7-methoxy-quinazoline, 4-[(3-chloro-4-fluoro-phenyl)amino]-6-[cis-4-(N-methanesulphonyl-N-methyl-amino)-cyclohexan-1-yloxy]-7-methoxy-quinazoline, 4-[(3-chloro-4-fluoro-phenyl)amino]-6-[cis-4-(N-acetyl-N-methyl-amino)-cyclohexan-1-yloxy]-7-methoxy-quinazoline, 4-[(3-chloro-4-fluoro-phenyl)amino]-6-(trans-4-methylamino-cyclohexan-1-yloxy)-7-methoxy-quinazoline, 4-[(3-chloro-4-fluoro-phenyl)amino]-6-[trans-4-(N-methanesulphonyl-N-methyl-amino)-cyclohexan-1-yloxy]-7-methoxy-quinazoline, 4-[(3-chloro-4-fluoro-phenyl)amino]-6-(trans-4-dimethylamino-cyclohexan- -yloxy)-7-methoxy-quinazoline, 4-[(3-chloro-4-fluoro-phenyl)amino]-6-(trans-4-{N-[(morpholin-4-yl)carbonyl]-N-methyl-amino}-cyclohexan-1-yloxy)-7-methoxy-quinazoline, 4-[(3-chloro-4-fluoro-phenyl)amino]-6-[2-(2,2-dimethyl-6-oxo-morpholin-4-yl)-ethoxy]-7-[(S)-(tetrahydrofuran-2-yl)methoxy]-quinazoline, 4-[(3-chloro-4-fluoro-phenyl)amino]-6-(1-methanesulphonyl-piperidin-4-yloxy)-7-methoxy-quinazoline, 4-[(3-chloro-4-fluoro-phenyl)amino]-6-(1-cyano-piperidin-4-yloxy)-7-methoxy-quinazoline, and cetuximab, optionally in the form of the racemates, enantiomers or diastereomers thereof, optionally in the form of the pharmacologically acceptable acid addition salts thereof, the solvates and/or hydrates thereof.

Particularly preferably, the EGFR-inhibitors 2e used within the scope of the medicament combinations according to the invention are selected from the group comprising 4-[(3-chloro-4-fluorophenyl)amino]-6-{[4-(morpholin-4-yl)-1-oxo-2-buten-1-yl]amino}-7-cyclopropylmethoxy-quinazoline, 4-[(R)-(1-phenyl-ethyl)amino]-6-{[4-(morpholin-4-yl)-1-oxo-2-buten-1-yl]amino}-7-cyclopentyloxy-quinazoline, 4-[(3-chloro-4-fluoro-phenyl)amino]-6-{[4-((R)-6-methyl-2-oxo-morpholin-4-yl)-1-oxo-2-buten-1-yl]amino}-7-[(S)-(tetrahydrofuran-3-yl)oxy]-quinazoline, 4-[(3-chloro-4-fluoro-phenyl)amino]-6-[2-((S)-6-methyl-2-oxo-morpholin-4-yl)-ethoxy]-7-methoxy-quinazoline, 4-[(3-chloro-4-fluorophenyl)amino]-6-({4-[N-(2-methoxy-ethyl)-N-methyl-amino]-1-oxo-2-buten-1-yl}amino)-7-cyclopropylmethoxy-quinazoline, 4-[(R)-(1-phenyl-ethyl)amino]-6-({4-[N-(tetrahydropyran-4-yl)-N-methyl-amino]-1-oxo-2-buten-1-yl}amino)-7-cyclopropylmethoxy-quinazoline, 4-[(3-chloro-4-fluorophenyl)amino]-6-({4-[N-(2-methoxy-ethyl)-N-methyl-amino]-1-oxo-2-buten-1-yl}amino)-7-cyclopentyloxy-quinazoline, 4-[(3-chloro-4-fluorophenyl)amino]-6-{[4-(N,N-dimethylamino)-1-oxo-2-buten-1-yl]amino}-7-[(R)-(tetrahydrofuran-2-yl)methoxy]-quinazoline, 4-[(3-ethynyl-phenyl)amino]-6,7-bis-(2-methoxy-ethoxy)-quinazoline, 4-[(R)-(1-phenyl-ethyl)amino]-6-(4-hydroxy-phenyl)-7H-pyrrolo[2,3-d]pyrimidine, 3-cyano-4-[(3-chloro-4-fluorophenyl)amino]-6-{[4-(N,N-dimethylamino)-1-oxo-2-buten-1-yl]amino}-7-ethoxy-quinoline, 4-[(R)-(1-phenyl-ethyl)amino]-6-{[4-((R)-6-methyl-2-oxo-morpholin-4-yl)-1-oxo-2-buten-1-yl]amino}-methoxy-quinazoline, 4-[(3-chloro-4-fluorophenyl)amino]-6-{[4-(morpholin-4-yl)-1-oxo-2-buten-1-yl]amino}-7-[(tetrahydrofuran-2-yl)methoxy]-quinazoline, 4-[(3-ethynyl-phenyl)amino]-6-{[4-(5,5-dimethyl-2-oxo-morpholin-4-yl)-1-oxo-2-buten-1-yl]amino}-quinazoline, 4-[(3-chloro-4-fluoro-phenyl)amino]-6-{2-[4-(2-oxo-morpholin-4-yl)-piperidin-1-yl]-ethoxy}-7-methoxy-quinazoline, 4-[(3-chloro-4-fluoro-phenyl)amino]-6-(trans-4-amino-cyclohexan-1-yloxy)-7-methoxy-quinazoline, 4-[(3-chloro-4-fluoro-phenyl)amino]-6-(trans-4-methanesulphonylamino-cyclohexan-1-yloxy)-7-methoxy-quinazoline, 4-[(3-chloro-4-fluoro-phenyl)amino]-6-(tetrahydropyran-3-yloxy)-7-methoxy-quinazoline, 4-[(3-chloro-4-fluoro-phenyl)amino]-6-{1-[(morpholin-4-yl)carbonyl]-piperidin-4-yloxy}-7-methoxy-quinazoline, 4-[(3-chloro-4-fluoro-phenyl)amino]-6-(piperidin-3-yloxy)-7-methoxy-quinazoline, 4-[(3-chloro-4-fluoro-phenyl)amino]-6-[1-(2-acetylamino-ethyl)-piperidin-4-yloxy]-7-methoxy-quinazoline, 4-[(3-chloro-4-fluoro-phenyl)amino]-6-(tetrahydropyran-4-yloxy)-7-ethoxy-quinazoline, 4-[(3-chloro-4-fluoro-phenyl)amino]-6-{trans-4-[(morpholin-4-yl)carbonylamino]-cyclohexan-1-yloxy}-7-methoxy-quinazoline, 4-[(3-chloro-4-fluoro-phenyl)amino]-6-{1-[(piperidin-1-yl)carbonyl]-piperidin-4-yloxy}-7-methoxy-quinazoline, 4-[(3-chloro-4-fluoro-phenyl)amino]-6-(cis-4-{N-[(morpholin-4-yl)carbonyl]-N-methyl-amino}-cyclohexan-1-yloxy)-7-methoxy-quinazoline, 4-[(3-chloro-4-fluoro-phenyl)amino]-6-(trans-4-ethanesulphonylamino-cyclohexan-1-yloxy)-7-methoxy-quinazoline, 4-[(3-chloro-4-fluoro-phenyl)amino]-6-(1-methanesulphonyl-piperidin-4-yloxy)-7-(2-methoxy-ethoxy)-quinazoline, 4-[(3-chloro-4-fluoro-phenyl)amino]-6-[1-(2-methoxy-acetyl)-piperidin-4-yloxy]-7-(2-methoxy-ethoxy)-quinazoline, 4-[(3-ethynyl-phenyl)amino]-6-(tetrahydropyran-4-yloxy]-7-methoxy-quinazoline, 4-[(3-chloro-4-fluoro-phenyl)amino]-6-(cis-4-{N-[(piperidin-1-yl)carbonyl]-N-methyl-amino}-cyclohexan-1-yloxy)-7-methoxy-quinazoline, 4-[(3-chloro-4-fluoro-phenyl)amino]-6-{cis-4-[(morpholin-4-yl)carbonylamino]-cyclohexan-1-yloxy}-7-methoxy-quinazoline, 4-[(3-chloro-4-fluoro-phenyl)amino]-6-{1-[2-(2-oxopyrrolidin-1-yl)ethyl]-piperidin-4-yloxy}-7-methoxy-quinazoline, 4-[(3-ethynyl-phenyl)amino]-6-(1-acetyl-piperidin-4-yloxy)-7-methoxy-quinazoline, 4-[(3-ethynyl-phenyl)amino]-6-(1-methyl-piperidin-4-yloxy)-7-methoxy-quinazoline, 4-[(3-ethynyl-phenyl)amino]-6-(1-methanesulphonyl-piperidin-4-yloxy)-7-methoxy-quinazoline, 4-[(3-chloro-4-fluoro-phenyl)amino]-6-(1-methyl-piperidin-4-yloxy)-7(2-methoxy-ethoxy)-quinazoline, 4-[(3-ethynyl-phenyl)amino]-6-{1-[(morpholin-4-yl)carbonyl]-piperidin-4-yloxy}-7-methoxy-quinazoline, 4-[(3-chloro-4-fluoro-phenyl)amino]-6-{1-[(N-methyl-N-2-methoxyethyl-amino)carbonyl]-piperidin-4-yloxy}-7-methoxy-quinazoline, 4-[(3-chloro-4-fluoro-phenyl)amino]-6-(1-ethyl-piperidin-4-yloxy)-7-methoxy-quinazoline, 4-[(3-chloro-4-fluoro-phenyl)amino]-6-[cis-4-(N-methanesulphonyl-N-methyl-amino)-cyclohexan-1-yloxy]-7-methoxy-quinazoline, 4-[(3-chloro-4-fluoro-phenyl)amino]-6-[cis-4-(N-acetyl-N-methyl-amino)-cyclohexan-1-yloxy]-7-methoxy-quinazoline, 4-[(3-chloro-4-fluoro-phenyl)amino]-6-(trans-4-methylamino-cyclohexan-1-yloxy)-7-methoxy-quinazoline, 4-[(3-chloro-4-fluoro-phenyl)amino]-6-[trans-4-(N-methanesulphonyl-N-methyl-amino)-cyclohexan-1-yloxy]-7-methoxy-quinazoline, 4-[(3-chloro-4-fluoro-phenyl)amino]-6-(trans-4-dimethylamino-cyclohexan-1-yloxy)-7-methoxy-quinazoline, 4-[(3-chloro-4-fluoro-phenyl)amino]-6-(trans-4-{N-[(morpholin-4-yl)carbonyl]-N-methyl-amino}-cyclohexan-1-yloxy)-7-methoxy-quinazoline, 4-[(3-chloro-4-fluoro-phenyl)amino]-6-[2-(2,2-dimethyl-6-oxo-morpholin-4-yl)-ethoxy]-7-[(S)-(tetrahydrofuran-2-yl)methoxy]-quinazoline, 4-[(3-chloro-4-fluoro-phenyl)amino]-6-(1-methanesulphonyl-piperidin-4-yloxy)-7-methoxy-quinazoline, 4-[(3-chloro-4-fluoro-phenyl)amino]-6-( 1-cyano-piperidin-4-yloxy)-7-methoxy-quinazoline, and 4-[(3-chloro-4-fluoro-phenyl)amino]-6-{1-[(2-methoxyethyl)carbonyl]-piperidin-4-yloxy}-7-methoxy-quinazoline, optionally in the form of the racemates, enantiomers or diastereomers thereof, optionally in the form of the pharmacologically acceptable acid addition salts thereof, the solvates and/or hydrates thereof.

Particularly preferred medicament combinations according to the invention contain as EGFR-inhibitors 2e those compounds which are selected from the group comprising
4-[(3-chloro-4-fluorophenyl)amino]-6-{[4-(morpholin-4-yl)-1-oxo-2-buten-1-yl]amino}-7-cyclopropylmethoxy-quinazoline (2e.1),
4-[(3-chloro-4-fluoro-phenyl)amino]-6-{[4-((R)-6-methyl-2-oxo-morpholin-4-yl)-1-oxo-2-buten-1-yl]amino}-7-[(S)-(tetrahydrofuran-3-yl)oxy]-quinazoline (2e.2),
4-[(3-chloro-4-fluoro-phenyl)amino]-6-[2-((S)-6-methyl-2-oxo-morpholin-4-yl)-ethoxy]-7-methoxy-quinazoline (2e.3),
4-[(3-chloro-4-fluorophenyl)amino]-6-({4-[N-(2-methoxy-ethyl)-N-methyl-amino]-1-oxo-2-buten-1-yl}amino)-7-cyclopropylmethoxy-quinazoline (2e.4),
4-[(3-ethynyl-phenyl)amino]-6,7-bis-(2-methoxy-ethoxy)-quinazoline (2e.5),
4-[(3-chloro-4-fluorophenyl)amino]-6-{[4-(morpholin-4-yl)-1-oxo-2-buten-1-yl]amino}-7-[(tetrahydrofuran-2-yl)methoxy]-quinazoline (2e.6),
4-[(3-ethynyl-phenyl)amino]-6-{[4-(5,5-dimethyl-2-oxo-morpholin-4-yl)-1-oxo-2-buten-1-yl]amino}-quinazoline (2e.7),
4-[(3-chloro-4-fluoro-phenyl)amino]-6-(trans-4-methanesulphonylamino-cyclohexan-1-yloxy)-7-methoxy-quinazoline (2e.8),
4-[(3-chloro-4-fluoro-phenyl)amino]-6-(tetrahydropyran-3-yloxy)-7-methoxy-quinazoline (2e.9),
4-[(3-chloro-4-fluoro-phenyl)amino]-6-{1-[(morpholin-4-yl)carbonyl]-piperidin-4-yloxy}-7-methoxy-quinazoline (2e.10),
4-[(3-chloro-4-fluoro-phenyl)amino]-6-{1-[2-(2-oxopyrrolidin-1-yl)ethyl]-piperidin-4-yloxy}-7-methoxy-quinazoline (2e.11),
4-[(3-ethynyl-phenyl)amino]-6-(1-acetyl-piperidin-4-yloxy)-7-methoxy-quinazoline (2e.12),
4-[(3-ethynyl-phenyl)amino]-6-(1-methyl-piperidin-4-yloxy)-7-methoxy-quinazoline (2e.1 3),
4-[(3-ethynyl-phenyl)amino]-6-(1-methanesulphonyl-piperidin-4-yloxy)-7-methoxy-quinazoline (2e.14),
4-[(3-ethynyl-phenyl)amino]-6-{1-[(morpholin-4-yl)carbonyl]-piperidin-4-yloxy}-7-methoxy-quinazoline (2e.15),
4-[(3-chloro-4-fluoro-phenyl)amino]-6-{1-[(2-methoxyethyl)carbonyl]-piperidin-4-yloxy}-7-methoxy-quinazoline (2e.16),
4-[(3-chloro-4-fluoro-phenyl)amino]-6-[cis-4-(N-methanesulphonyl-N-methyl-amino)-cyclohexan-1-yloxy]-7-methoxy-quinazoline (2e.17),
4-[(3-chloro-4-fluoro-phenyl)amino]-6-[cis-4-(N-acetyl-N-methyl-amino)-cyclohexan-1-yloxy]-7-methoxy-quinazoline (2e.18),
4-[(3-chloro-4-fluoro-phenyl)amino]-6-(trans-4-methylamino-cyclohexan-1-yloxy)-7-methoxy-quinazoline (2e.19),
4-[(3-chloro-4-fluoro-phenyl)amino]-6-[trans-4-(N-methanesulphonyl-N-methyl-amino)-cyclohexan-1-yloxy]-7-methoxy-quinazoline (2e.20),
4-[(3-chloro-4-fluoro-phenyl)amino]-6-(trans-4-dimethylamino-cyclohexan-1-yloxy)-7-methoxy-quinazoline (2e.21),
4-[(3-chloro-4-fluoro-phenyl)amino]-6-(trans-4-{N-[(morpholin-4-yl)carbonyl]-N-methyl-amino}-cyclohexan-1-yloxy)-7-methoxy-quinazoline (2e.22),
4-[(3-chloro-4-fluoro-phenyl)amino]-6-[2-(2,2-dimethyl-6-oxo-morpholin-4-yl)-ethoxy]-7-[(S)-(tetrahydrofuran-2-yl)methoxy]-quinazoline (2e.23),
4-[(3-chloro-4-fluoro-phenyl)amino]-6-(1-methanesulphonyl-piperidin-4-yloxy)-7-methoxy-quinazoline (2e.24) and
4-[(3-chloro-4-fluoro-phenyl)amino]-6-(1-cyano-piperidin-4-yloxy)-7-methoxy-quinazoline (2e.25),
   optionally in the form of the racemates, enantiomers or diastereomers thereof, optionally in the form of the pharmacologically acceptable acid addition salts thereof, the solvates and/or hydrates thereof.

By the acid addition salts with pharmacologically acceptable acids which the compounds 2e may possibly be capable of forming are meant for example salts selected from the group comprising the hydrochloride, hydrobromide, hydriodide, hydrosulphate, hydrophosphate, hydromethanesulphonate, hydronitrate, hydromaleate, hydroacetate, hydrobenzoate, hydrocitrate, hydrofumarate, hydrotartrate, hydroxalate, hydrosuccinate, hydrobenzoate and hydro-p-toluenesulphonate, preferably the hydrochloride, hydrobromide, hydrosulphate, hydrophosphate, hydrofumarate and hydromethanesulphonate.

Examples of novel preferred medicament combinations of preferred compounds of formula 1 with the above-mentioned EGFR-inhibitors 2e are combinations containing the compounds 1.2 and 2e.1; 1.2 and 2e.2; 1.2 and 2e.3; 1.2 and 2e.4; 1.2 and 2e.5; 1.2 and 2e.6; 1.2 and 2e.7; 1.2 and 2e.8; 1.2 and 2e.9; 1.2 and 2e.10; 1.2 and 2e.11; 1.2 and 2e.12; 1.2 and 2e.13; 1.2 and 2e.14; 1.2 and 2e.15; 1.2 and 2e.16; 1.2 and 2e.17; 1.2 and 2e.18; 1.2 and 2e.19; 1.2 and 2e.20; 1.2 and 2e.21; 1.2 and 2e.22; 1.2 and 2e.23; 1.2 and 2e.24; 1.2 and 2e.25; 1.3 and 2e.1; 1.3 and 2e.2; 1.3 and 2e.3; 1.3 and 2e.4; 1.3 and 2e.5; 1.3 and 2e.6; 1.3 and 2e.7; 1.3 and 2e.8; 1.3 and 2e.9; 1.3 and 2e.10; 1.3 and 2e.11; 1.3 and 2e.12; 1.3 and 2e.13; 1.3 and 2e.14; 1.3 and 2e.15; 1.3 and 2e.16; 1.3 and 2e.17; 1.3 and 2e.18; 1.3 and 2e.19; 1.3 and 2e.20; 1.3 and 2e.21; 1.3 and 2e.22; 1.3 and 2e.23; 1.3 and 2e.24; 1.3 and 2e.25; 1.6 and 2e.1; 1.6 and 2e.2; 1.6 and 2e.3; 1.6 and 2e.4; 1.6 and 2e.5; 1.6 and 2e.6; 1.6 and 2e.7; 1.6 and 2e.8; 1.6 and 2e.9; 1.6 and 2e.10; 1.6 and 2e.11; 1.6 and 2e.12; 1.6 and 2e.13; 1.6 and 2e.14; 1.6 and 2e.15; 1.6 and 2e.16; 1.6 and 2e.17; 1.6 and 2e.18; 1.6 and 2e.19; 1.6 and 2e.20; 1.6 and 2e.21; 1.6 and 2e.22; 1.6 and 2e.23; 1.6 and 2e.24; 1.6 and 2e.25; 1.7 and 2e.1; 1.7 and 2e.2; 1.7 and 2e.3; 1.7 and 2e.4; 1.7 and 2e.5; 1.7 and 2e.6; 1.7 and 2e.7; 1.7 and 2e.8; 1.7 and 2e.9; 1.7 and 2e.10; 1.7 and 2e.11; 1.7 and 2e.12; 1.7 and 2e.13; 1.7 and 2e.14; 1.7 and 2e.15; 1.7 and 2e.16; 1.7 and 2e.17; 1.7 and 2e.18; 1.7 and 2e.19; 1.7 and 2e.20; 1.7 and 2e.21; 1.7 and 2e.22; 1.7 and 2e.23; 1.7 and 2e.24; 1.7 and 2e.25; 1.9 and 2e.1; 1.9 and 2e.2; 1.9 and 2e.3; 1.9 and 2e.4; 1.9 and 2e.5; 1.9 and 2e.6; 1.9 and 2e.7; 1.9 and 2e.8; 1.9 and 2e.9; 1.9 and 2e.10; 1.9 and 2e.11; 1.9 and 2e.12; 1.9 and 2e.13; 1.9 and 2e.14; 1.9 and 2e.15; 1.9 and 2e.16; 1.9 and 2e.17; 1.9 and 2e.18; 1.9 and 2e.19; 1.9 and 2e.20; 1.9 and 2e.21; 1.9 and 2e.22; 1.9 and 2e.23; 1.9 and 2e.24; 1.9 and 2e.25; in each case optionally in the form of the racemates, enantiomers or diastereomers thereof and optionally in the form of the pharmacologically acceptable acid addition salts, solvates and/or hydrates thereof. Of the above-mentioned combinations the preferred ones according to the invention are also those which contain as compound 2e one of the compounds 2e.1, 2e.2, 2e.3, 2e.4, 2e.10, 2e.11, 2e.14, 2e.16, 2e.17, 2e.18, 2e.19, 2e.20, 2e.21, 2e.22, 2e.23, 2e.24 or 2e.25, while those combinations that contain one of the compounds 2e.2, 2e.3 or 2e.4 are of particular importance according to the invention.

The novel medicament combinations comprising compounds of formula 1 with at least one other active substance 2 are not restricted to binary combinations of active substances. The combinations mentioned above, partly by way of example, which contain in addition to a compound of formula 1 one other active substance 2, may also contain a third or fourth, preferably a third active substance, which is also selected from the above-mentioned group of anticholinergics (2a), PDE IV inhibitors (2b), steroids (2c), LTD4-antagonists (2d) and EGFR-inhibitors (2e).

Particularly preferred combinations which contain two other active substances in addition to a compound of formula 1 are selected from the active substance combinations listed below. These are medicament combinations which may contain, for example

A) a compound of formula 1, an anticholinergic (2a), a PDE IV inhibitor (2b);

B) a compound of formula 1, an anticholinergic (2a), a steroid (2c);

C) a compound of formula 1, an anticholinergic (2a), an LTD4 antagonist (2d);

D) a compound of formula 1, an anticholinergic (2a), an EGFR inhibitor (2e);

E) a compound of formula 1, a PDEIV inhibitor (2b), a steroid (2c);

F) a compound of formula 1, a PDEIV inhibitor (2b), an LTD4 antagonist (2d);

G) a compound of formula 1, a PDEIV inhibitor (2b), an EGFR inhibitor (2e);

H) a compound of formula 1, a steroid (2c), an LTD4 antagonist (2d);

I) a compound of formula 1, a steroid (2c), an EGFR inhibitor (2e);

J) a compound of formula 1, an LTD4 antagonist (2d), an EGFR inhibitor (2e).

Of outstanding importance according to the invention are all those medicament combinations disclosed within the scope of the present invention which contain the compounds of formula 1 in the form of the R-enantiomers thereof.

Terms and Definitions Used

Within the scope of the present invention, by a medicament combination of components 1 and 2 is meant the joint administration of both active substances in a single preparation or formulation or the separate administration of the two active substances in separate formulations. If the active substances 1 and 2 are administered in separate formulations, this separate administration may be done simultaneously or at different times, i.e. successively. In one aspect the present invention relates to the above-mentioned medicament combinations which contain in addition to therapeutically effective amounts of 1 and 2 a pharmaceutically acceptable carrier. In one aspect the present invention relates to the above-mentioned pharmaceutical compositions which do not contain a pharmaceutically acceptable carrier in addition to therapeutically effective amounts of 1 and 2.

By the term “C₁₋₆-alkyl” (including those which are part of other groups) are meant branched and unbranched alkyl groups with 1 to 6 carbon atoms and by the term “C₁₋₄-alkyl” are meant branched and unbranched alkyl groups with 1 to 4 carbon atoms. Alkyl groups with 1 to 4 carbon atoms are preferred. Examples include: methyl, ethyl, n-propyl, iso-propyl, n-butyl, iso-butyl, sec-butyl, tert-butyl, n-pentyl, iso-pentyl, neo-pentyl or hexyl. The abbreviations Me, Et, n-Pr, i-Pr, n-Bu, i-Bu, t-Bu, etc. may optionally also be used for the above-mentioned groups. Unless stated otherwise, the definitions propyl, butyl, pentyl and hexyl include all the possible isomeric forms of the groups in question. Thus, for example, propyl includes n-propyl and iso-propyl, butyl includes iso-butyl, sec-butyl and tert-butyl etc.

By the term “C₂₋₆-alkenyl” (including those which are part of other groups) are meant branched and unbranched alkenyl groups with 2 to 6 carbon atoms and by the term “C₂₋₄-alkenyl” are meant branched and unbranched alkenyl groups with 2 to 4 carbon atoms, provided that they have at least one double bond. Alkenyl groups with 2 to 4 carbon atoms are preferred. Examples of these include: ethenyl or vinyl, propenyl, butenyl, pentenyl, or hexenyl. Unless stated otherwise, the definitions propenyl, butenyl, pentenyl and hexenyl include all the possible isomeric forms of the groups in question.

Thus, for example, propenyl includes 1-propenyl and 2-propenyl, butenyl includes 1-, 2- and 3-butenyl, 1-methyl-1-propenyl, 1-methyl-2-propenyl etc.

By the term “C₂₋₆-alkynyl” (including those which are part of other groups) are meant branched and unbranched alkynyl groups with 2 to 6 carbon atoms and by the term “C₂₋₄-alkynyl” are meant branched and unbranched alkynyl groups with 2 to 4 carbon atoms, provided that they have at least one triple bond. Alkynyl groups with 2 to 4 carbon atoms are preferred. Examples of these include: ethynyl, propynyl, butynyl, pentynyl or hexynyl. Unless stated otherwise, the definitions propynyl, butynyl, pentynyl and hexynyl include all the possible isomeric forms of the groups in question. Thus for example propynyl includes 1-propynyl and 2-propynyl, butynyl includes 1, 2- and 3-butynyl, 1-methyl-1-propynyl, 1-methyl-2-propynyl etc.

By the term “C₅₋₆-cycloalkyl” (including those which are part of other groups) are meant cyclic alkyl groups with 5 or 6 carbon atoms. Examples of these include: cyclopentyl or cyclohexyl. Unless otherwise stated, the cyclic alkyl groups may be substituted by one or more groups selected from among methyl, ethyl, iso-propyl, tert-butyl, hydroxy, fluorine, chlorine, bromine and iodine.

By the term “C₁₋₆-haloalkyl” (including those which are part of other groups) are meant branched and unbranched alkyl groups with 1 to 6 carbon atoms, which are substituted by one or more halogen atoms. By the term “C₁₋₄-alkyl” are meant branched and unbranched alkyl groups with 1 to 4 carbon atoms, which are substituted by one or more halogen atoms. Alkyl groups with 1 to 4 carbon atoms are preferred. Preferred halogen atoms are fluorine, chlorine, particularly preferably fluorine. Examples of these include: CF₃, CHF₂, CH₂F, CH₂CF₃.

Halogen within the scope of the present invention represents fluorine, chlorine, bromine or iodine. Unless stated to the contrary, fluorine, chlorine and bromine are regarded as preferred halogens.

Compounds of general formula 1 may have acid groups, predominantly carboxyl groups, and/or basic groups such as e.g. amino functions. Compounds of general formula 1 may therefore be in the form of internal salts, salts with pharmaceutically acceptable inorganic acids such as hydrochloric acid, sulphuric acid, phosphoric acid, sulphonic acid or organic acids (such as for example maleic acid, fumaric acid, citric acid, tartaric acid or acetic acid) or as salts with pharmaceutically acceptable bases such as alkali or alkaline earth metal hydroxides or carbonates, zinc or ammonium hydroxides or organic amines such as e.g. diethylamine, triethylamine, triethanolamine etc.

By acid addition salts with pharmacologically acceptable acids of the compounds 1 are meant for example salts selected from among hydrochloride, hydrobromide, hydriodide, hydrosulphate, hydrophosphate, hydromethanesulphonate, hydronitrate, hydromaleate, hydroacetate, hydrobenzoate, hydrocitrate, hydrofumarate, hydrotartrate, hydroxalate, hydrosuccinate, hydrobenzoate and hydro-p-toluenesulphonate, preferably hydrochloride, hydrobromide, hydrosulphate, hydrophosphate, hydrofumarate and hydromethanesulphonate. Of the above-mentioned acid addition salts, the salts of hydrochloric acid, methanesulphonic acid, benzoic acid and acetic acid are particularly preferred according to the invention.

Indications

The present invention also relates to the use of therapeutically effective amounts of the active substances 1 for preparing a pharmaceutical composition also containing one or more, preferably one active substance 2 for the treatment of inflammatory and obstructive respiratory complaints, for inhibiting premature labour in midwifery (tocolysis), for restoring sinus rhythm in the heart in atrioventricular block, for correcting bradycardic heart rhythm disorders (antiarrhythmic), for treating circulatory shock (vasodilatation and increasing the heart volume) as well as for the treatment of skin irritations and inflammation.

In a preferred aspect the present invention relates to the use of therapeutically effective amounts of the active substances 1 for preparing a pharmaceutical composition also containing one or more, preferably one, active substance 2 for the treatment of respiratory complaints selected from the group comprising obstructive pulmonary diseases of various origins, pulmonary emphysema of various origins, restrictive pulmonary diseases, interstitial pulmonary diseases, cystic fibrosis, bronchitis of various origins, bronchiectasis, ARDS (adult respiratory distress syndrome) and all forms of pulmonary oedema.

Preferably the medicament combinations according to the invention are used as specified above for preparing a pharmaceutical composition for the treatment of obstructive pulmonary diseases selected from among bronchial asthma, paediatric asthma, severe asthma, acute asthma attacks, chronic bronchitis and COPD (chronic obstructive pulmonary disease), while it is particularly preferable according to the invention to use them for preparing a pharmaceutical composition for the treatment of bronchial asthma and COPD.

It is also preferable to use the medicament combinations according to the invention for preparing a pharmaceutical composition for the treatment of pulmonary emphysema which has its origins in COPD (chronic obstructive pulmonary disease) or α1-proteinase inhibitor deficiency.

It is also preferable to use the medicament combinations according to the invention for preparing a pharmaceutical composition for the treatment of restrictive pulmonary diseases selected from among allergic alveolitis, restrictive pulmonary diseases triggered by work-related noxious substances, such as asbestosis or silicosis, and restriction caused by lung tumours, such as for example lymphangiosis carcinomatosa, bronchoalveolar carcinoma and lymphomas.

It is also preferable to use the medicament combinations according to the invention for preparing a pharmaceutical composition for the treatment of interstitial pulmonary diseases selected from among pneumonia caused by infections, such as for example infection by viruses, bacteria, fungi, protozoa, helminths or other pathogens, pneumonitis caused by various factors, such as for example aspiration and left heart insufficiency, radiation-induced pneumonitis or fibrosis, collagenoses, such as for example lupus erythematodes, systemic sclerodermy or sarcoidosis, granulomatoses, such as for example Boeck's disease, idiopathic interstitial pneumonia or idiopathic pulmonary fibrosis (IPF).

It is also preferable to use the medicament combinations according to the invention for preparing a pharmaceutical composition for the treatment of cystic fibrosis or mucoviscidosis.

It is also preferable to use the medicament combinations according to the invention for preparing a pharmaceutical composition for the treatment of bronchitis, such as for example bronchitis caused by bacterial or viral infection, allergic bronchitis and toxic bronchitis.

It is also preferable to use the medicament combinations according to the invention for preparing a pharmaceutical composition for the treatment of bronchiectasis.

It is also preferable to use the medicament combinations according to the invention for preparing a pharmaceutical composition for the treatment of ARDS (adult respiratory distress syndrome).

It is also preferable to use the medicament combinations according to the invention for preparing a pharmaceutical composition for the treatment of pulmonary oedema, for example toxic pulmonary oedema after aspiration or inhalation of toxic substances and foreign substances.

It is particularly preferable to use the compounds detailed above for preparing a pharmaceutical composition for the treatment of asthma or COPD. Also of particular importance is the above-mentioned use of medicament combinations according to the invention for preparing a pharmaceutical composition for once-a-day treatment of inflammatory and obstructive respiratory complaints, particularly for the once-a-day treatment of asthma or COPD.

Formulation

The present invention also relates to the use of therapeutically effective amounts of an active substance of formula 1 in combination with therapeutically effective amounts of an active substance 2 for preparing a pharmaceutical composition for the treatment of one of the above-mentioned diseases.

The present invention also relates to a process for treating one of the above-mentioned diseases, which is characterised in that therapeutically effective amounts of active substance of formula 1 are administered in combination with therapeutically effective amounts of active substance 2.

Within the scope of the medicament combinations according to the invention, for example, 0.1-1000 μg of a compound of formula 1 may be administered per single dose.

Preferably, 1-500 μg, particularly preferably 3-100 μg of the compound of formula 1 are administered per single dose, while a dosage range of from 5-75 μg, preferably from 7-50 μg is preferred according to the invention. Particularly preferably, the pharmaceutical compositions according to the invention are administered in an amount such that 9-40 μg, particularly preferably 11-30 μg, more preferably 12-25 μg of the compound of formula 1 are administered per single dose. For example, and without restricting the present invention thereto, 5 μg, 7.5 μg, 10 μg, 12.5 μg, 15 μg, 17.5 μg, 20 μg, 22.5 μg, 25 μg, 27.5 μg, 30 μg, 32.5 μg, 35 μg, 37.5 μg, 40 μg, 42.5 μg, 45 μg, 47.5 μg, 50 μg, 52.5 μg, 55 μg, 57.5 μg, 60 μg, 62.5 μg, 65 μg, 67.5 μg, 70 μg, 72.5 μg or 75 μg of a compound of formula 1 may be administered per single dose.

The above-mentioned dosages relate to the compounds of formula 1 in the form of their free bases. If the compounds of formula 1 are administered in the form of their pharmaceutically acceptable acid addition salts, the skilled man can easily calculate the corresponding dosage ranges for the acid addition salts from the dosage ranges specified above, taking into account the molecular weight of the acids used. Particularly preferably, the compounds of formula 1 are administered in the above-mentioned dosage ranges in the form of the enantiomerically pure compounds, particularly preferably in the form of the R-enantiomers thereof.

If the compounds of formula 1 are administered in conjunction with an anticholinergic 2a, the amount of anticholinergic used will fluctuate considerably depending on the choice of active substance.

Without restricting the invention thereto, in the case of tiotropium 2a.1′ amounts of anticholinergic (2a.1′) may be administered such that each single dose contains 0.1-80 μg, preferably 0.5-60 μg, particularly preferably about 1-50 μg of 2a.1′. For example and without restricting the present invention thereto, 2.5 μg, 5 μg, 10 μg, 18 μg, 20 μg, 36 μg or 40 μg 2a.1′ may be administered per single dose. The corresponding amount of salt 2a.1 or of any hydrate or solvate used in each case can easily be calculated by the skilled man, depending on the choice of anion. If for example tiotropium bromide is used as the preferred tiotropium salt 2a.1 according to the invention, the amounts of the active substance 2a.1′ administered per single dose as specified by way of example hereinbefore correspond to the following amounts of 2a.1 administered per single dose: 3 μg, 6 μg, 12 μg, 21.7 μg, 24.1 μg, 43.3 μg and 48.1 μg of 2a.1. In the case of tiotropium 2a.1′ the dosages specified above are preferably administered once or twice a day, while administration once a day is particularly preferred according to the invention.

Without restricting the invention thereto, in the case of the cation 2a.2′ amounts of anticholinergic (2a.2′) may be administered such that each single dose contains 1-500 μg, preferably 5-300 μg, particularly preferably 15-200 μg 2a.2′. For example and without restricting the present invention thereto, 15 μg, 20 μg, 25 μg, 30 μg, 35 μg, 40 μg, 45 μg, 50 μg, 55 μg, 60 μg, 65 μg, 70 μg, 75 μg, 80 μg, 85 μg, 90 μg, 95 μg, 100 μg, 105 μg, 110 μg, 115 μg, 120 μg, 125 μg, 130 μg, 135 μg, 140 μg, 145 μg, 150 μg, 155 μg, 160 μg, 165 μg, 170 μg, 175 μg, 180 μg, 185 μg, 190 μg, 195 μg or 200 μg of 2a.2′ may be administered per single dose. The corresponding amount of salt 2a.2 used in each case or of any hydrate or solvate used can easily be calculated by the skilled man, depending on the choice of anion. In the case of oxitropium 2a.2′ the dosages specified above are preferably administered one to four times a day, while administration two to three times a day is particularly preferred according to the invention.

Without restricting the invention thereto, in the case of the cation 2a.3′ amounts of anticholinergic (2a.3′) may be administered such that each single dose contains 1-500 μg, preferably 5-300 μg, particularly preferably 15-200 μg 2a.3′. For example and without restricting the present invention thereto, 15 μg, 20 μg, 25 μg, 30 μg, 35 μg, 40 μg, 45 μg, 50 μg, 55 μg, 60 μg, 65 μg, 70 μg, 75 μg, 80 μg, 85 μg, 90 μg, 95 μg, 100 μg, 105 μg, 110 μg, 115 μg, 120 μg, 125 μg, 130 μg, 135 μg, 140 μg, 145 μg, 150 μg, 155 μg, 160 μg, 165 μg, 170 μg, 175 μg, 180 μg, 185 μg, 190 μg, 195 μg or 200 μg of 2a.3′ may be administered per single dose. The corresponding amount of salt 2a.3 used in each case or of any hydrate or solvate used can easily be calculated by the skilled man, depending on the choice of anion. In the case of flutropium 2a.3′ the dosages specified above are preferably administered one to four times a day, while administration two to three times a day is particularly preferred according to the invention.

Without restricting the invention thereto, in the case of the cation 2a.4′ amounts of anticholinergic (2a.4′) may be administered such that each single dose contains 1-500 μg, preferably 5-300 μg, particularly preferably 20-200 μg 2a.4′. For example and without restricting the present invention thereto, 20 μg, 25 μg, 30 μg, 35 μg, 40 μg, 45 μg, 50 μg, 55 μg, 60 μg, 65 μg, 70 μg, 75 μg, 80 μg, 85 μg, 90 μg, 95 μg, 100 μg, 105 μg, 110 μg, 115 μg, 120 μg, 125 μg, 130 μg, 135 μg, 140 μg, 145 μg, 150 μg, 155 μg, 160 μg, 165 μg, 170 μg, 175 μg, 180 μg, 185 μg, 190 μg, 195 μg or 200 μg of 2a.4′ may be administered per single dose. The corresponding amount of salt 2a.4 used in each case or of any hydrate or solvate used can easily be calculated by the skilled man, depending on the choice of anion. In the case of ipratropium 2a.4′ the dosages specified above are preferably administered one to four times a day, while administration two to three times a day, more preferably three times a day, is particularly preferred according to the invention.

Without restricting the invention thereto, in the case of the cation 2a.5′ amounts of anticholinergic (2a.5′) may be administered such that each single dose contains 1-500 μg, preferably 5-300 μg, particularly preferably 15-200 μg. For example and without restricting the present invention thereto, 15 μg, 20 μg, 25 μg, 30 μg, 35 μg, 40 μg, 45 μg, 50 μg, 55 μg, 60 μg, 65 μg, 70 μg, 75 μg, 80 μg, 85 μg, 90 μg, 95 μg, 100 μg, 105 μg, 110 μg, 115 μg, 120 μg, 125 μg, 130 μg, 135 μg, 140 μg, 145 μg, 150 μg, 155 μg, 160 μg, 165 μg, 170 μg, 175 μg, 180 μg, 185 μg, 190 μg, 195 μg or 200 μg of 2a.5′ may be administered per single dose. The corresponding amount of salt 2a.5 used in each case or of any hydrate or solvate used can easily be calculated by the skilled man, depending on the choice of anion. In the case of glycopyrronium 2a.5′ the dosages specified above are preferably administered one to four times a day, while administration two to three times a day is particularly preferred according to the invention.

Without restricting the invention thereto, in the case of the cation 2a.6′ amounts of anticholinergic (2a.6′) may be administered such that each single dose contains 1000-6500 μg, preferably 2000-6000 μg, particularly preferably 3000-5500 μg, particularly preferably 4000-5000 μg 2a.6′. For example and without restricting the present invention thereto, 3500 μg, 3750 μg, 4000 μg, 4250 μg, 4500 μg, 4750 μg, or 5000 μg of 2a.6′ may be administered per single dose. The corresponding amount of salt 2a.6 used in each case or of any hydrate or solvate used can easily be calculated by the skilled man, depending on the choice of anion. In the case of trospium 2a.6′ the dosages specified above are preferably administered one to four times a day, while administration two to three times a day is particularly preferred according to the invention.

Without restricting the invention thereto, in the case of the cation 2a.7′ amounts of anticholinergic (2a.7′) may be administered such that each single dose contains 50-1000 μg, preferably 100-800 μg, particularly preferably 200-700 μg, particularly preferably 300-600 μg 2a.7′. For example and without restricting the present invention thereto, 300 μg, 350 μg, 400 μg, 450 μg, 500 μg, 550 μg, or 600 μg of 2a.7′ may be administered per single dose. The corresponding amount of salt 2a.7 used in each case or of any hydrate or solvate used can easily be calculated by the skilled man, depending on the choice of anion. In the case of the cation 2a.7′ the dosages specified above are preferably administered one to three times a day, while administration once or twice a day, more preferably once a day, is particularly preferred according to the invention.

Without restricting the invention thereto, in the case of the cations 2a.9′ and 2a.10′, amounts of anticholinergic (2a.9′ or 2a.10′) may be administered such that each single dose contains 1-500 μg, preferably 5-300 μg, particularly preferably 15-200 μg 2a.9′ or 2a.10′. For example and without restricting the present invention thereto, 15 μg, 20 μg, 25 μg, 30 μg, 35 μg, 40 μg, 45 μg, 50 μg, 55 μg, 60 μg, 65 μg, 70 μg, 75 μg, 80 μg, 85 μg, 90 μg, 95 μg, 100 μg, 105 μg, 110 μg, 115 μg, 120 μg, 125 μg, 130 μg, 135 μg, 140 μg, 145 μg, 150 μg, 155 μg, 160 μg, 165 μg, 170 μg, 175 μg, 180 μg, 185 μg, 190 μg, 195 μg, or 200 μg of 2a.9′ or 2a.10′ may be administered per single dose. The corresponding amount of salt 2a.9′ or 2a.10′ or of any hydrate or solvate used in each case can easily be calculated by the skilled man, depending on the choice of anion. In the case of the cations 2a.9′ or 2a.10′ the dosages specified above are preferably administered one to three times a day, while administration once or twice a day, more preferably once a day, is particularly preferred according to the invention.

Without restricting the invention thereto, in the case of the cations 2a.11′ to 2a.13′ amounts of anticholinergic (2a.11′, 2a.12′ or 2a.13′) may be administered such that each single dose contains 1-500 μg, preferably 5-300 μg, particularly preferably 10-200 μg 2a.11′, 2a.12′ or 2a.13′. For example and without restricting the present invention thereto, 10 μg, 15 μg, 20 μg, 25 μg, 30 μg, 35 μg, 40 μg, 45 μg, 50 ∥g, 55 μg, 60 μg, 65 μg, 70 μg, 75 μg, 80 μg, 85 μg, 90 μg, 95 μg, 100 μg, 105 μg, 110 μg, 115 μg, 120 μg, 125 μg, 130 μg, 135 μg, 140 μg, 145 μg, 150 μg, 155 μg, 160 μg, 165 μg, 170 μg, 175 μg, 180 μg, 185 μg, 190 μg, 195 μg or 200 μg of 2a.11′, 2a.12′ or 2a.13′ may be any per single dose. The corresponding amount of salt 2a.11, 2a.12 or 2a.13 or of any hydrate or solvate used in each case can easily be calculated by the skilled man, depending on the choice of anion.

In the case of the cations 2a.11, 2a.12 or 2a.13 the dosages specified above are preferably administered one to three times a day, while administration once or twice a day, more preferably once a day, is particularly preferred according to the invention.

If the compounds of formula 1 are administered in combination with a PDE IV-inhibitor 2b, preferably about 1-10000 μg 2b are administered per single dose. Preferably, amounts of 2b are administered such that each single dose contains 10-5000 μg, preferably 50-2500 μg, particularly preferably 100-1000 μg of 2b. For example and without restricting the present invention thereto, 100 μg, 115 μg, 120 μg, 125 μg, 130 μg, 135 μg, 140 μg, 145 μg, 150 μg, 155 μg, 160 μg, 165 μg, 170 μg, 175 μg, 180 μg, 185 μg, 190 μg, 195 μg, 200 μg, 205 μg, 210 μg, 215 μg, 220 μg, 225 μg, 230 μg, 235 μg, 240 μg, 245 μg, 250 μg, 255 μg, 260 μg, 265 μg, 270 μg, 275 μg, 280 μg, 285 μg, 290 μg, 295 μg, 300 μg, 305 μg, 310 μg, 315 μg, 320 μg, 325 μg, 330 μg, 335 μg, 340 μg, 345 μg, 350 μg, 355 μg, 360 μg, 365 μg, 370 μg, 375 μg, 380 μg, 385 μg, 390 μg, 395 μg, 400 μg, 405 μg, 410 μg, 415 μg, 420 μg, 425 μg, 430 μg, 435 μg, 440 μg, 445 μg, 450 μg, 455 μg, 460 μg, 465 μg, 470 μg, 475 μg, 480 μg, 485 μg, 490 μg, 495 μg, 500 μg, 505 μg, 510 μg, 515 μg, 520 μg, 525 μg, 530 μg, 535 μg, 540 μg, 545 μg, 550 μg, 555 μg, 560 μg, 565 μg, 570 μg, 575 μg, 580 μg, 585 μg, 590 μg, 595 μg, 600 μg, 605 μg, 610 μg, 615 μg, 620 μg, 625 μg, 630 μg, 635 μg, 640 μg, 645 μg, 650 μg, 655 μg, 660 μg, 665 μg, 670 μg, 675 μg, 680 μg, 685 μg, 690 μg, 695 μg, 700 μg, 705 μg, 710 μg, 715 μg, 720 μg, 725 μg, 730 μg, 735 μg, 740 μg, 745 μg, 750 μg, 755 μg, 760 μg, 765 μg, 770 μg, 775 μg, 780 μg, 785 μg, 790 μg, 795 μg, 800 μg, 805 μg, 810 μg, 815 μg, 820 μg, 825 μg, 830 μg, 835 μg, 840 μg, 845 μg, 850 μg, 855 μg, 860 μg, 865 μg, 870 μg, 875 μg, 880 μg, 885 μg, 890 μg, 895 μg, 900 μg, 905 μg, 910 μg, 915 μg, 920 μg, 925 μg, 930 μg, 935 μg, 940 μg, 945 μg, 950 μg, 955 μg, 960 μg, 965 μg, 970 μg, 975 μg, 980 μg, 985 μg, 990 μg, 995 μg or 1000 μg of 2b may be administered per single dose. In the event that acid addition salts of 2b are used, the corresponding amount of salt used can easily be calculated by the skilled man from the values given hereinbefore, depending on the choice of acid.

If the compounds of formula 1 are administered in combination with a steroid 2c, preferably about 1-10000 μg of 2c are administered per single dose. Preferably, amounts of 2c are administered such that each single dose contains 5-5000 μg, preferably 5-2500 μg, particularly preferably 10-1000 μg of 2c. For example and without restricting the present invention thereto, 10 μg, 15 μg, 20 μg, 25 μg, 30 μg, 35 μg, 40 μg, 45 μg, 50 μg, 55 μg, 60 μg, 65 μg, 70 μg, 75 μg, 80 μg, 85 μg, 90 μg, 95 μg, 100 μg, 115 μg, 120 μg, 125 μg, 130 μg, 135 μg, 140 μg, 145 μg, 150 μg, 155 μg, 160 μg,165 μg, 170 μg, 175 μg, 180 μg, 185 μg, 190 μg, 195 μg, 200 μg, 205 μg, 210 μg, 215 μg, 220 μg, 225 μg, 230 μg, 235 μg, 240 μg, 245 μg, 250 μg, 255 μg, 260 μg, 265 μg, 270 μg, 275 μg, 280 μg, 285 μg, 290 μg, 295 μg, 300 μg, 305 μg, 310 μg, 315 μg, 320 μg, 325 μg, 330 μg, 335 μg, 340 μg, 345 μg, 350 μg, 355 μg, 360 μg, 365 μg, 370 μg, 375 μg, 380 μg, 385 μg, 390 μg, 395 μg, 400 μg, 405 μg, 410 μg, 415 μg, 420 μg, 425 μg, 430 μg, 435 μg, 440 μg, 445 μg, 450 μg, 455 μg, 460 μg, 465 μg, 470 μg, 475 μg, 480 μg, 485 μg, 490 μg, 495 μg, 500 μg, 505 μg, 510 μg, 515 μg, 520 μg, 525 μg, 530 μg, 535 μg, 540 μg, 545 μg, 550 μg, 555 μg, 560 μg, 565 μg, 570 μg, 575 μg, 580 μg, 585 μg, 590 μg, 595 μg, 600 μg, 605 μg, 610 μg, 615 μg, 620 μg, 625 μg, 630 μg, 635 μg, 640 μg, 645 μg, 650 μg, 655 μg, 660 μg, 665 μg, 670 μg, 675 μg, 680 μg, 685 μg, 690 μg, 695 μg, 700 μg, 705 μg, 710 μg, 715 μg, 720 μg, 725 μg, 730 μg, 735 μg, 740 μg, 745 μg, 750 μg, 755 μg, 760 μg, 765 μg, 770 μg, 775 μg, 780 μg, 785 μg, 790 μg, 795 μg, 800 μg, 805 μg, 810 μg, 815 μg, 820 μg, 825 μg, 830 μg, 835 μg, 840 μg, 845 μg, 850 μg, 855 μg, 860 μg, 865 μg, 870 μg, 875 μg, 880 μg, 885 μg, 890 μg, 895 μg, 900 μg, 905 μg, 910 μg, 915 μg, 920 μg, 925 μg, 930 μg, 935 μg, 940 μg, 945 μg, 950 μg, 955 μg, 960 μg, 965 μg, 970 μg, 975 μg, 980 μg, 985 μg, 990 μg, 995 μg or 1000 μg of 2c may be administered per single dose. In the event that salts or derivatives of 2c are used, the corresponding amount of salt/derivative used can easily be calculated by the skilled man from the values given hereinbefore, depending on the choice of salt/derivative.

If the compounds of formula 1 are administered in combination with an LTD4-antagonist 2d, preferably about 0.01-500 mg 2d are administered per single dose. Preferably, amounts of 2d are administered such that each single dose contains 0.1-250 mg, preferably 0.5-100 mg, particularly preferably 1-50 mg of 2d. For example and without restricting the present invention thereto, 1 mg, 2.5 mg, 5 mg, 5.5 mg, 7 mg, 7, 5 mg, 10 mg, 12.5 mg, 15 mg, 17.5 mg, 20 mg, 22.5 mg, 25 mg, 27.5 mg, 30 mg, 32.5 mg, 35 mg, 37.5 mg, 40 mg, 42.5 mg, 45 mg, 47.5 mg or 50 mg of 2d may be administered per single dose. In the event that acid addition salts, salts or derivatives of 2d are used, the corresponding amount of salt/derivative used can easily be calculated by the skilled man from the values given hereinbefore, depending on the choice of salt/derivative. # If the compounds of formula 1 are administered in combination with an EGFR-inhibitor 2e, preferably about 100-15000 μg of 2e are administered per single dose. Preferably, amounts of 2e are administered such that each single dose contains 500-10000 μg, preferably 750-8000 μg, particularly preferably 1000-7000 μg of 2e. For example and without restricting the present invention thereto, 1000 μg, 1150 μg, 1200 μg, 1250 μg, 1300 μg, 1350 μg, 1400 μg, 1450 μg, 1500 μg, 1550 μg, 1600 μg, 1650 μg, 1700 μg, 1750 μg, 1800 μg, 1850 μg, 1900 μg, 1950 μg, 2000 μg, 2050 μg, 2100 μg, 2150 μg, 2200 μg, 2250 μg, 2300 μg, 2350 μg, 2400 μg, 2450 μg, 2500 μg, 2550 μg, 2600 μg, 2650 μg, 2700 μg, 2750 μg, 2800 μg, 2850 μg, 2900 μg, 2950 μg, 3000 μg, 3050 μg, 3100 μg, 3150 μg, 3200 μg, 3250 μg, 3300 μg, 3350 μg, 3400 μg, 3450 μg, 3500 μg, 3550 μg, 3600 μg, 3650 μg, 3700 μg, 3750 μg, 3800 μg, 3850 μg, 3900 μg, 3950 μg, 4000 μg, 4050 μg, 4100 μg, 4150 μg, 4200 μg, 4250 μg, 4300 μg, 4350 μg, 4400 μg, 4450 μg, 4500 μg, 4550 μg, 4600 μg, 4650 μg, 4700 μg, 4750 μg, 4800 μg, 4850 μg, 4900 μg, 4950 μg, 5000 μg, 5050 μg, 5100 μg, 5150 μg, 5200 μg, 5250 μg, 5300 μg, 5350 μg, 5400 μg, 5450 μg, 5500 μg, 5550 μg, 5600 μg, 5650 μg, 5700 μg, 5750 μg, 5800 μg, 5850 μg, 5900 μg, 5950 μg, 6000 μg, 6050 μg, 6100 μg, 6150 μg, 6200 μg, 6250 μg, 6300 μg, 6350 μg, 6400 μg, 6450 μg, 6500 μg, 6550 μg, 6600 μg, 6650 μg, 6700 μg, 6750 μg, 6800 μg, 6850 μg, 6900 μg, 6950 μg, or 700 μg of 2e may be administered per single dose. In the event that acid addition salts of 2e are used, the corresponding amount of the salt used can easily be calculated by the skilled man from the values given hereinbefore, depending on the choice of acid.

The two active substance components 1 and 2 may be administered—together or separately—in each case by inhalation or by oral, parenteral or some other route, in known manner, in substantially conventional formulations such as for example plain or coated tablets, pills, granules, aerosols, syrups, emulsions, suspensions, powders and solutions, using inert, non-toxic, pharmaceutically suitable carriers or solvents.

Suitable preparations for administering the compounds of formula 1 and 2 include tablets, capsules, suppositories, solutions, powders, etc. The proportion of pharmaceutically active compound or compounds should be in the range from 0.05 to 90% by weight, preferably 0.1 to 50% by weight of the total composition. Suitable tablets may be obtained, for example, by mixing the active substance(s) with known excipients, for example inert diluents such as calcium carbonate, calcium phosphate or lactose, disintegrants such as corn starch or alginic acid, binders such as starch or gelatine, lubricants such as magnesium stearate or talc and/or agents for delaying release, such as carboxymethyl cellulose, cellulose acetate phthalate, or polyvinyl acetate. The tablets may also comprise several layers.

Coated tablets may be prepared accordingly by coating cores produced analogously to the tablets with substances normally used for tablet coatings, for example collidone or shellac, gum arabic, talc, titanium dioxide or sugar. To achieve delayed release or prevent incompatibilities the core may also consist of a number of layers. Similarly the tablet coating may consist of a number or layers to achieve delayed release, possibly using the excipients mentioned above for the tablets.

Syrups or elixirs containing the active substances or combinations of active substances according to the invention may additionally contain a sweetener such as saccharine, cyclamate, glycerol or sugar and a flavour enhancer, e.g. a flavouring such as vanillin or orange extract. They may also contain suspension adjuvants or thickeners such as sodium carboxymethyl cellulose, wetting agents such as, for example, condensation products of fatty alcohols with ethylene oxide, or preservatives such as p-hydroxybenzoates.

Solutions are prepared in the usual way, e.g. with the addition of isotonic agents, preservatives such as p-hydroxybenzoates, or stabilisers such as alkali metal salts of ethylenediamine tetraacetic acid, optionally using emulsifiers and/or dispersants, whilst if water is used as the diluent, for example, organic solvents may optionally be used as solvating agents or dissolving aids, and transferred into injection vials or ampoules or infusion bottles.

Capsules containing one or more active substances or combinations of active substances may for example be prepared by mixing the active substances with inert carriers such as lactose or sorbitol and packing them into gelatine capsules. Suitable suppositories may be made for example by mixing with carriers provided for this purpose, such as neutral fats or polyethyleneglycol or the derivatives thereof. Excipients which may be used include, for example, water, pharmaceutically acceptable organic solvents such as paraffins (e.g. petroleum fractions), vegetable oils (e.g. groundnut or sesame oil), mono- or polyfunctional alcohols (e.g. ethanol or glycerol), carriers such as e.g. natural mineral powders (e.g. kaolins, clays, talc, chalk), synthetic mineral powders (e.g. highly dispersed silicic acid and silicates), sugars (e.g. cane sugar, lactose and glucose), emulsifiers (e.g. lignin, spent sulphite liquors, methylcellulose, starch and polyvinylpyrrolidone) and lubricants (e.g. magnesium stearate, talc, stearic acid and sodium lauryl sulphate).

For oral administration the tablets may, of course, contain, apart from the abovementioned carriers, additives such as sodium citrate, calcium carbonate and dicalcium phosphate together with various additional substances such as starch, preferably potato starch, gelatine and the like. Moreover, lubricants such as magnesium stearate, sodium lauryl sulphate and talc may be used at the same time for the tabletting process. In the case of aqueous suspensions the active substances may be combined with various flavour enhancers or colourings in addition to the excipients mentioned above.

Preferably, even when the two components 1 and 2 are administered separately, at least component 1 is administered by inhalation. If component 1 is administered by inhalation, when the two active substances are taken separately, component 2 may also be administered for example by oral or parenteral route using formulations conventional in the art such as plain or coated tablets, pills, granules, aerosols, syrups, emulsions, suspensions, powders and solutions, using inert, non-toxic, pharmaceutically suitable carriers or solvents.

Preferably, however, the medicament combinations according to the invention are administered by inhalation by means of a single preparation containing both active substances 1 and 2 or by means of separate preparations each containing only one of the active substances 1 and 2, suitable for administration by inhalation.

Inhalable preparations include inhalable powders, propellant-containing metered dose aerosols or propellant-free inhalable solutions. Inhalable powders according to the invention containing the combination of active substances 1 and 2 may consist of the active substances on their own or of a mixture of the active substances with physiologically acceptable excipients. Within the scope of the present invention, the term propellant-free inhalable solutions also includes concentrates or sterile inhalable solutions ready for use. The preparations according to the invention may contain the combination of active substances 1 and 2 either together in one formulation or in two separate formulations. These formulations which may be used within the scope of the present invention are described in more detail in the next part of the specification.

A) Inhalable Powder Containing the Combinations of Active Substances According to the Invention:

The inhalable powders according to the invention may contain 1 and 2 either on their own or in admixture with suitable physiologically acceptable excipients. If the active substances 1 and 2 are present in admixture with physiologically acceptable excipients, the following physiologically acceptable excipients may be used to prepare these inhalable powders according to the invention: monosaccharides (e.g. glucose or arabinose), disaccharides (e.g. lactose, saccharose, maltose, trehalose), oligo- and polysaccharides (e.g. dextrans), polyalcohols (e.g. sorbitol, mannitol, xylitol), salts (e.g. sodium chloride, calcium carbonate) or mixtures of these excipients with one another. Preferably, mono- or disaccharides are used, while the use of lactose, trehalose or glucose is preferred, particularly, but not exclusively, in the form of their hydrates.

Within the scope of the inhalable powders according to the invention the excipients have a maximum average particle size of up to 250 μm, preferably between 10 and 150 μm, most preferably between 15 and 80 μm. It may sometimes seem appropriate to add finer excipient fractions with an average particle size of 1 to 9 μm to the excipients mentioned above. These finer excipients are also selected from the group of possible excipients listed hereinbefore. Finally, in order to prepare the inhalable powders according to the invention, micronised active substance 1 and 2, preferably with an average particle size of 0.5 to 10 μm, more preferably from 1 to 6 μm, is added to the excipient mixture. Processes for producing the inhalable powders according to the invention by grinding and micronising and finally mixing the ingredients together are known from the prior art. The inhalable powders according to the invention may be prepared and administered either in the form of a single powder mixture which contains both 1 and 2 or in the form of separate inhalable powders which contain only 1 or 2.

The inhalable powders according to the invention may be administered using inhalers known from the prior art. Inhalable powders according to the invention which contain a physiologically acceptable excipient in addition to 1 and 2 may be administered, for example, by means of inhalers which deliver a single dose from a supply using a measuring chamber as described in U.S. Pat. No. 4,570,630A, or by other means as described in DE 36 25 685 A. The inhalable powders according to the invention which contain 1 and 2 optionally in conjunction with a physiologically acceptable excipient may be administered, for example, using the inhaler known by the name Turbohaler® or using inhalers as disclosed for example in EP 237507 A. Preferably, the inhalable powders according to the invention which contain physiologically acceptable excipients in addition to 1 and 2 are packed into capsules (to produce so-called inhalettes) which are used in inhalers as described, for example, in WO 94/28958.

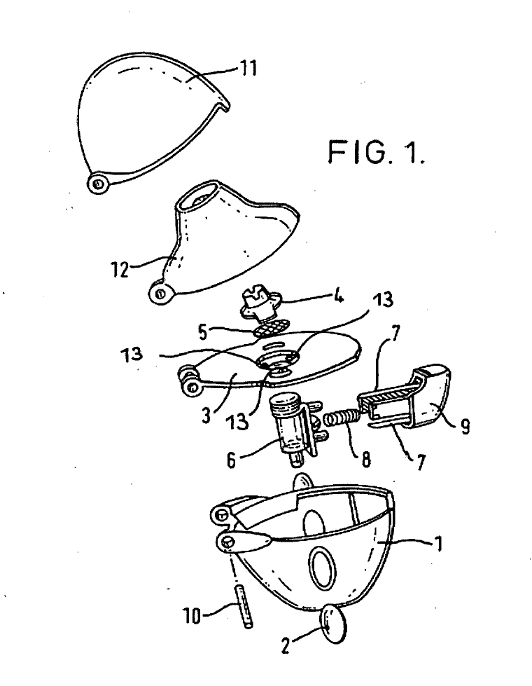

A particularly preferred inhaler for using the pharmaceutical combination according to the invention in inhalettes is shown in FIG. 1. This inhaler (Handihaler®) for inhaling powdered pharmaceutical compositions from capsules is characterised by a housing 1 containing two windows 2, a deck 3 in which there are air inlet ports and which is provided with a screen 5 secured by a screen housing 4, an inhalation chamber 6 connected to the deck 3 on which there is a push button 9 provided with two sharpened pins 7 and movable counter to a spring 8, and a mouthpiece 12 which is connected to the housing 1, the deck 3 and a cover 11 via a spindle 10 to enable it to be flipped open or shut, and air through-holes 13 for adjusting the flow resistance.

If the inhalable powders according to the invention are to be packaged in capsules, in accordance with the preferred method of administration described above, the capsules should preferably contain from 1 to 30 mg each. According to the invention they contain either together or separately the dosages per single dose specified for 1 and 2 hereinbefore.

B) Propellant Gas-Driven Inhalation Aerosols Containing the Combinations of Active Substances According to the Invention:

Inhalation aerosols containing propellant gas according to the invention may contain substances 1 and 2 dissolved in the propellant gas or in dispersed form. 1 and 2 may be present in separate formulations or in a single preparation, in which 1 and 2 are either both dissolved, both dispersed or only one component is dissolved and the other is dispersed. The propellant gases which may be used to prepare the inhalation aerosols according to the invention are known from the prior art. Suitable propellant gases are selected from among hydrocarbons such as n-propane, n-butane or isobutane and halohydrocarbons such as preferably chlorinated and fluorinated derivatives of methane, ethane, propane, butane, cyclopropane or cyclobutane. The propellant gases mentioned above may be used on their own or in mixtures thereof. Particularly preferred propellant gases are halogenated alkane derivatives selected from TG11, TG12, TG134a (1,1,1,2-tetrafluoroethane), TG227 (1,1,1,2,3,3,3-heptafluoropropane) and mixtures thereof, the propellant gases TG134a, TG227 and mixtures thereof being preferred.

The propellant-driven inhalation aerosols according to the invention may also contain other ingredients such as co-solvents, stabilisers, surfactants, antioxidants, lubricants and pH adjusters. All these ingredients are known in the art.

The inhalation aerosols containing propellant gas according to the invention may contain up to 5 wt.-% of active substance 1 and/or 2. Aerosols according to the invention contain, for example, 0.002 to 5 wt.-%, 0.01 to 3 wt.-%, 0.015 to 2 wt.-%, 0.1 to 2 wt.-%, 0.5 to 2 wt.-% or 0.5 to 1 wt.-% of active substance 1 and/or 2.

If the active substances 1 and/or 2 are present in dispersed form, the particles of active substance preferably have an average particle size of up to 10 μm, preferably from 0.1 to 6 μm, more preferably from 1 to 5 μm.

The propellant-driven inhalation aerosols according to the invention mentioned above may be administered using inhalers known in the art (MDIs=metered dose inhalers). Accordingly, in another aspect, the present invention relates to pharmaceutical compositions in the form of propellant-driven aerosols as hereinbefore described combined with one or more inhalers suitable for administering these aerosols. In addition, the present invention relates to inhalers which are characterised in that they contain the propellant gas-containing aerosols described above according to the invention.

The present invention also relates to cartridges which are fitted with a suitable valve and can be used in a suitable inhaler and which contain one of the above-mentioned propellant gas-containing inhalation aerosols according to the invention. Suitable cartridges and methods of filling these cartridges with the inhalable aerosols containing propellant gas according to the invention are known from the prior art.

C) Propellant-Free Inhalable Solutions or Suspensions Containing the Combinations of Active Substances 1 and 2 According to the Invention:

Propellant-free inhalable solutions according to the invention contain for example aqueous or alcoholic, preferably ethanolic solvents, possibly ethanolic solvents in admixture with aqueous solvents. In the case of aqueous/ethanolic solvent mixtures the relative proportion of ethanol to water is not restricted, but the maximum limit is up to 70 percent by volume, more particularly up to 60 percent by volume of ethanol. The remainder of the volume is made up of water. The solutions or suspensions containing 1 and 2, separately or together, are adjusted to a pH of 2 to 7, preferably 2 to 5, using suitable acids. The pH may be adjusted using acids selected from inorganic or organic acids. Examples of particularly suitable inorganic acids include hydrochloric acid, hydrobromic acid, nitric acid, sulphuric acid and/or phosphoric acid. Examples of particularly suitable organic acids include ascorbic acid, citric acid, malic acid, tartaric acid, maleic acid, succinic acid, fumaric acid, acetic acid, formic acid and/or propionic acid, etc. Preferred inorganic acids are hydrochloric acid and sulphuric acid. It is also possible to use the acids which have already formed an acid addition salt with one of the active substances. Of the organic acids, ascorbic acid, fumaric acid and citric acid are preferred. If desired, mixtures of the above acids may also be used, particularly in the case of acids which have other properties in addition to their acidifying qualities, e.g. as flavourings, antioxidants or complexing agents, such as citric acid or ascorbic acid, for example. According to the invention, it is particularly preferred to use hydrochloric acid to adjust the pH.

According to the invention, the addition of edetic acid (EDTA) or one of the known salts thereof, sodium edetate, as stabiliser or complexing agent is unnecessary in the present formulation. Other embodiments may contain this compound or these compounds. In a preferred embodiment the content based on sodium edetate is less than 100 mg/100 ml, preferably less than 50 mg/100 ml, more preferably less than 20 mg/100 ml. Generally, inhalable solutions in which the content of sodium edetate is from 0 to 10 mg/100 ml are preferred.

Co-solvents and/or other excipients may be added to the propellant-free inhalable solutions according to the invention. Preferred co-solvents are those which contain hydroxyl groups or other polar groups, e.g. alcohols—particularly isopropyl alcohol, glycols—particularly propyleneglycol, polyethyleneglycol, polypropyleneglycol, glycolether, glycerol, polyoxyethylene alcohols and polyoxyethylene fatty acid esters. The terms excipients and additives in this context denote any pharmacologically acceptable substance which is not an active substance but which can be formulated with the active substance or substances in the pharmacologically suitable solvent in order to improve the qualitative properties of the active substance formulation. Preferably, these substances have no pharmacological effect or, in connection with the desired therapy, no appreciable or at least no undesirable pharmacological effect. The excipients and additives include, for example, surfactants such as soya lecithin, oleic acid, sorbitan esters, such as polysorbates, polyvinylpyrrolidone, other stabilisers, complexing agents, antioxidants and/or preservatives which guarantee or prolong the shelf life of the finished pharmaceutical formulation, flavourings, vitamins and/or other additives known in the art. The additives also include pharmacologically acceptable salts such as sodium chloride as isotonic agents.

The preferred excipients include antioxidants such as ascorbic acid, for example, provided that it has not already been used to adjust the pH, vitamin A, vitamin E, tocopherols and similar vitamins and provitamins occurring in the human body.

Preservatives may be used to protect the formulation from contamination with pathogens. Suitable preservatives are those which are known in the art, particularly cetyl pyridinium chloride, benzalkonium chloride or benzoic acid or benzoates such as sodium benzoate in the concentration known from the prior art. The preservatives mentioned above are preferably present in concentrations of up to 50 mg/100 ml, more preferably between 5 and 20 mg/100 ml.

Preferred formulations contain, in addition to the solvent water and the combination of active substances 1 and 2, only benzalkonium chloride and sodium edetate. In another preferred embodiment, no sodium edetate is present.

The propellant-free inhalable solutions according to the invention are administered in particular using inhalers of the kind which are capable of nebulising a small amount of a liquid formulation in the therapeutic dose within a few seconds to produce an aerosol suitable for therapeutic inhalation. Within the scope of the present invention, preferred inhalers are those in which a quantity of less than 100 μL, preferably less than 50 μL, more preferably between 10 and 30 μL of active substance solution can be nebulised in preferably one spray action to form an aerosol with an average particle size of less than 20 μm, preferably less than 10 μm, such that the inhalable part of the aerosol corresponds to the therapeutically effective quantity.

An apparatus of this kind for propellant-free delivery of a metered quantity of a liquid pharmaceutical composition for inhalation is described for example in International Patent Application WO 91/14468 and also in WO 97/12687 (cf. in particular FIGS. 6a and 6b). The nebulisers (devices) described therein are known by the name Respimat®.

The above-mentioned examples of the active substances 2 are known in the art. The compounds of formula 1 by contrast are not known in the art.

The examples of synthesis described hereinafter serve to illustrate possible methods of synthesising the new compounds of formula 1. However, they are intended only as examples of procedures as an illustration of the invention without restricting the invention to the subject-matter described by way of example.

The compounds according to the invention may be prepared analogously to the methods already known in the art. Suitable methods of preparation are known for example U.S. Pat. Nos. 4,460,581 and 4,154,829 bekannt, the contents of which are hereby incorporated by reference.

### Examples

Synthesis of the Intermediate Products

### Intermediate Product 1: 1,1-dimethyl-3-(5-methyl-3-p-tolyl-[1,2,4]triazol-1-yl)-propylamine

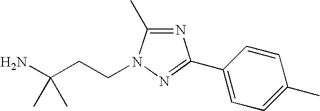

a) 4-methyl-benzoic acid-(1-imino-ethyl)-hydrazide:1.65 g (72 mmol) sodium are dissolved in 80 mL ethanol. 8.89 g (72 mmol) ethylacetimidate hydrochloride in 160 mL ethanol are added at ambient temperature and the sodium chloride precipitated is filtered off. The filtrate is combined with 6.00 g (40 mmol) 4-methyl-benzoic acid hydrazide and stirred overnight. The reaction mixture is evaporated down and cooled. The precipitated solid is filtered off and washed with cold ethanol and diethyl ether (5.7 g). After the solvents have been distilled off and the residue recrystallised from ethanol a further 1.2 g solid are obtained from the filtrate. Yield: 6.93 g (91%); mass spectroscopy [M+H]⁺=192.

b) 5-methyl-3-p-tolyl-[1,2,4]triazole: 7.58 g (40 mmol) 4-methyl-benzoic acid-(1-imino-ethyl)-hydrazide are heated to 180° C. for 30 minutes with stirring. After cooling the solid is dissolved in chloroform. The precipitate that settles out on cooling is suction filtered and recrystallised from chloroform. Yield: 4.82 g (70%); mass spectroscopy [M+H]⁺=174.

c) tert-butyl[1,1-dimethyl-3-(5-methyl-3-p-tolyl-[1,2,4]triazol-1-yl)-propyl]-carbamate: 1.35 g (34 mmol, 60%) sodium hydride are added at 0° C. to a solution of 4.87 g (28 mmol) 5-methyl-3-p-tolyl-[1,2,4]triazole in 40 mL DMPU. The reaction mixture is heated to ambient temperature and then stirred for one hour. 9.35 g (42 mmol) tert-butyl(3-chloro-1,1-dimethyl-propyl)-carbamate and 1.87 g (5 mmol) tetrabutylammonium iodide are added and the mixture is stirred overnight at ambient temperature and then again for 2 hours at 80° C. Water and ethyl acetate are added, the aqueous phase is separated off and extracted with ethyl acetate. The combined organic phases are washed with water and sodium chloride solution, dried on sodium sulphate and evaporated down. The residue is purified by column chromatography (silica gel; petroleum ether/ethyl acetate=1:1). Oil. Yield: 2.97 g (30%); mass spectroscopy [M+H]⁺=359.

d) 1,1-dimethyl-3-(5-methyl-3-p-tolyl-[1,2,4]triazol-1-yl)-propylamine: A total of 11 mL trifluoroacetic acid are added dropwise to a solution of 2.97 g (8.3 mmol) tert-butyl[1,1-dimethyl-3-(5-methyl-3-p-tolyl-[1,2,4]triazol-1-yl)-propyl]-carbamate in 80 mL dichloromethane and the mixture is stirred overnight at ambient temperature. The solvent is distilled off and the residue is combined with diethyl ether and stirred. The precipitated solid is filtered off and washed. Yield: 2.11 g (68%, trifluoroacetate); mass spectroscopy [M+H]⁺=259.

### Intermediate Product 2: 3-[3-(4-fluoro-phenyl)-5-methyl-[1,2,4]triazol-1-yl]-1,1-dimethyl-propylamine

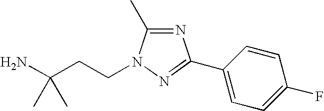

a) 4-fluoro-benzoic acid-(1-imino-ethyl)-hydrazide: prepared from 7.2 g (58 mmol) ethylacetimidate hydrochloride and 5.00 g (32 mmol) 4-fluoro-benzoic acid hydrazide analogously to the method described for intermediate product 1a). Yield: 5.78 g (91%); mass spectroscopy [M+H]⁺=196.

b) 3-(4-fluoro-phenyl)-5-methyl-[1,2,4]triazole: The preparation is carried out analogously to the method used for intermediate product 1b) from 5.77 g (30 mmol) 4-fluoro-benzoic acid-(1-imino-ethyl)-hydrazide. Yield: 4.11 g (78%); mass spectroscopy [M+H]⁺=178.

c) tert-butyl{3-[3-(4-fluoro-phenyl)-5-methyl-[1,2,4]triazol-1-yl]-1,1-dimethyl-propyl}-carbamate: 5.88 g (33 mmol) 3-(4-fluoro-phenyl)-5-methyl-[1,2,4]triazole are dissolved in 40 mL DMPU and reacted in the manner described for intermediate product 1c) with 11.04 g (50 mmol) tert-butyl(3-chloro-1,1-dimethyl-propyl)-carbamate, 1.59 g (40 mmol, 60%) sodium hydride and 2.21 g (6 mmol) tetrabutylammonium iodide. Yield: 4.22 g (35%); mass spectroscopy [M+H]⁺=363.

d) 3-[3-(4-fluoro-phenyl)-5-methyl-[1,2,4]triazol-1-yl]-1,1-dimethyl-propylamine: obtained by reacting 4.22 g (11.6 mmol) tert-butyl{3-[3-(4-fluoro-phenyl)-5-methyl-[1,2,4]triazol-1-yl]-1,1-dimethyl-propyl}-carbamate in 100 mL dichloromethane and 15 mL trifluoroacetic acid. Yield: 4.43 g (trifluoroacetate); mass spectroscopy [M+H]⁺=263.

### Intermediate Product 3: 3-[3-(3,5-difluoro-phenyl)-5-methyl-[1,2,4]triazol-1-yl]-1,1-dimethyl-propylamine

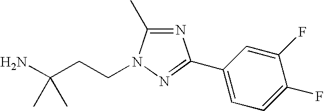

a) 3,5-difluoro-benzoic acid-(1-imino-ethyl)-hydrazide: obtained from 4.91 g (40 mmol) ethylacetimidate hydrochloride and 3.80 g (22 mmol) 3,5-difluoro-benzoic acid hydrazide analogously to the method described for intermediate product 1a). Yield: 4.49 g (95%); mass spectroscopy [M+H]⁺=214.

b) 3-(3,5-difluoro-phenyl)-5-methyl-[1,2,4]triazole: prepared from 4.61 g (22 mmol) 3,5-difluoro-benzoic acid-(1-imino-ethyl)-hydrazide. Yield: 3.81 g (91%); mass spectroscopy [M+H]⁺=196.

c) tert-butyl{3-[3-(3,5-difluoro-phenyl)-5-methyl-[1,2,4]triazol-1-yl]-1,1-dimethyl-propyl}-carbamate: 3.74 g (19 mmol) 3-(3,5-difluoro-phenyl)-5-methyl-[1,2,4]triazole in 25 mL DMPU are reacted with 0.92 g (23 mmol, 60%) sodium hydride, 6.37 g (29 mmol) tert-butyl(3-chloro-1,1-dimethyl-propyl)-carbamate and 1.27 g (3.5 mmol) tetrabutylammonium iodide analogously to intermediate product 1c). Yield: 2.62 g (36%); mass spectroscopy [M+H]⁺=381.

d) 3-[3-(3,5-difluoro-phenyl)-5-methyl-[1,2,4]triazol-1-yl]-1,1-dimethyl-propylamine: 2.62 g (6.9 mmol) tert-butyl{3-[3-(3,5-difluoro-phenyl)-5-methyl-[1,2,4]triazol-1-yl]-11-dimethyl-propyl}-carbamate in 65 mL dichloromethane are reacted with 9 mL trifluoroacetic acid in the manner described for intermediate product 1d). Yield: 2.11 g (trifluoroacetate); mass spectroscopy [M+H]⁺=281.

### Intermediate Product 4: 3-[5-ethyl-3-(4-methoxy-phenyl)-[1,2,4]triazol-1-yl]-1,1-dimethyl-propylamine

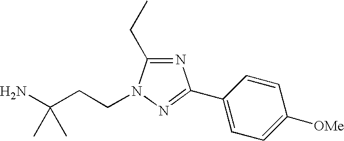

a) 4-methoxy-benzoic acid-(1-imino-propyl)-hydrazide: prepared from 4.90 g (45 mmol) propioamidine hydrochloride and 5.00 g (30 mmol) 4-methoxy-benzoic acid hydrazide analogously to the method described for intermediate product 1a). After the ethanol has been distilled off 10.0 g crude product are obtained, which are reacted without further purification.

b) 5-ethyl-3-(4-methoxy-phenyl)-[1,2,4]triazole: 9.99 g (60%, approx. 28 mmol) 4-methoxy-benzoic acid-(1-imino-propyl)-hydrazide are heated to 150° C. for two hours. After cooling the melt is purified by chromatography on a silica gel column (petroleum ether/ethyl acetate=3:7). Yield: 4.56 g (75% over two steps); mass spectroscopy [M+H]⁺=204.

c) tert-butyl{3-[5-ethyl-3-(4-methoxy-phenyl)-[1,2,4]triazol-1-yl]-1,1-dimethyl-propyl-carbamate: 4.30 g (21.2 mmol) 5-ethyl-3-(4-methoxy-phenyl)-[1,2,4]triazole are dissolved in 30 mL DMPU and cooled to 0° C. Under a protective gas atmosphere, 1.02 g (24 mmol, 60%) sodium hydride are then added batchwise and the reaction mixture is slowly heated to ambient temperature and then stirred for one hour. 6.10 g (27.5 mmol) tert-butyl(3-chloro-1,1-dimethyl-propyl)-carbamate and 1.41 g (3.8 mmol) tetrabutylammonium iodide are added. The mixture is stirred overnight and then the reaction is ended by the addition of water and ethyl acetate. The aqueous phase is separated off and extracted with ethyl acetate. The combined organic phases are washed with sodium chloride solution, dried on sodium sulphate and evaporated down. The residue is purified by chromatography on a silica gel column (petroleum ether/ethyl acetate=3:7). Yield: 6.82 g (83%); mass spectroscopy [M+H]⁺=389.

d) 3-[5-ethyl-3-(4-methoxy-phenyl)-[1,2,4]triazol-1-yl]-1,1-dimethyl-propylamine: A total of 20 mL trifluoroacetic acid are added dropwise to a solution of 6.81 g (17.5 mmol) tert-butyl{3-[5-ethyl-3-(4-methoxy-phenyl)-[1,2,4]triazol-1-yl]-1,1-dimethyl-propyl-carbamate in 150 mL dichloromethane. After three hours stirring at ambient temperature the solution is evaporated down and the residue is combined with diethyl ether. The precipitated solid is filtered off, washed with diethyl ether and dried. Yield: 7.86 g (trifluoroacetate); mass spectroscopy [M+H]⁺=289.

### Intermediate Product 5: methyl 3-[1-(3-amino-3-methyl-butyl)-5-methyl-1H-[1,2,4]triazol-3-yl]-benzoate

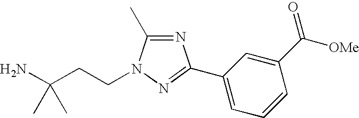

a) Methyl 3-[N′-benzyloxycarbonyl-hydrazinocarbonyl)-benzoate: 10.80 g (54.4 mmol) methyl 3-chlorocarbonyl-benzoate in 100 mL diethyl ether are added dropwise to a solution of 9.04 g (54.4 mmol) benzyl hydrazinecarboxylate in 100 mL diethyl ether, 100 mL dichloromethane and 4.83 mL pyridine while being cooled with an ice bath. The reaction mixture is stirred overnight at ambient temperature and then mixed with water. The precipitated solid is filtered off and washed with diethyl ether. Yield: 14.1 g (79%); mass spectroscopy [M−H]⁺=327.

b) methyl 3-hydrazinocarbonyl-benzoate: 14.6 g (44.5 mmol) methyl 3-[N′-benzyloxycarbonyl-hydrazinocarbonyl)-benzoate are dissolved in 75 mL methanol and hydrogenated in the presence of palladium on charcoal (10%) at ambient temperature and 3 bar hydrogen pressure. The catalyst is filtered off and the filtrate is freed from the solvent. Yield: 7.98 g (92%); mass spectroscopy [M+H]⁺=195.

c) methyl 3-[N′-(1-imino-ethyl)-hydrazinocarbonyl]-benzoate: prepared analogously to the method described for intermediate product 1a) from methyl 3-hydrazinocarbonyl-benzoate and ethylacetimidate hydrochloride. Yield: 8.60 g (90%); mass spectroscopy [M+H]⁺=236.

d) methyl 3-(5-methyl-1H-[1.24]triazol-3-yl)-benzoate: 8.10 g (34.4 mmol) methyl 3-[N′-(1-imino-ethyl)-hydrazinocarbonyl]-benzoate are heated to 180° C. for 30 minutes. 80 mL chloroform are added to the solid obtained after cooling. The suspension is filtered and the product is dried. Yield: 4.03 g (55%); mass spectroscopy [M+H]⁺=218.

e) methyl 3-[1-(3-tert-butoxycarbonylamino-3-methyl-butyl)-5-methyl-1H-[1,2,4]triazol-3-yl-benzoate: 6.00 g (27.6 mmol) methyl 3-(5-methyl-1H-[1.24]triazol-3-yl)-benzoate and 9.19 g (41.4 mmol) tert-butyl(3-chloro-1,1-dimethyl-propyl)-carbamate are reacted and worked up in the manner described for intermediate product 1c). Yield: 5.96 g (54%); mass spectroscopy [M+H]⁺=403.

f) methyl 3-[1-(3-amino-3-methyl-butyl)-5-methyl-1H-[1,2,4]triazol-3-yl]-benzoate: obtained from methyl 3-[1-(3-tert-butoxycarbonylamino-3-methyl-butyl)-5-methyl-1H-[1,2,4]triazol-3-yl-benzoate analogously to the method described for intermediate product 1d). Yield: 5.36 g (68%, ditrifluoroacetate); mass spectroscopy [M+H]⁺=303.

### Intermediate product 6: methyl 3-[1-(3-amino-3-methyl-butyl)-5-methyl-1H-[1,2,4]triazol-3-yl]-benzoate

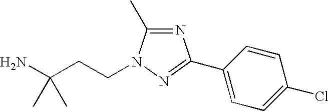

a) 4-chloro-benzoic acid N′-(1-imino-ethyl)-hydrazide: 1.09 g (20 mmol) sodium methoxide in 20 mL ethanol are added to a solution of 1.91 g (20 mmol) acetamidine hydrochloride in 30 mL ethanol. The mixture is stirred for 30 minutes at ambient temperature and then filtered. The filtrate is combined with 2.3 g (13.5 mmol) 4-chlorobenzoic acid hydrazide, stirred overnight at ambient temperature, cooled with an ice bath and then filtered. The precipitate is washed with cold ethanol and dried. Yield: 1.45 g (51%); mass spectroscopy [M+H]⁺=212/214.

b) 3-(4-chloro-phenyl)-5-methyl-1H-[1.24]triazole: 6.10 g (28.8 mmol) 4-chloro-benzoic acid N′-(1-imino-ethyl)-hydrazide are heated for 30 minutes to 180° C. After cooling 2.3 g product are obtained from the residue by recrystallisation in chloroform. Evaporation of the mother liquor and subsequent purification of the residue by chromatography (silica gel, petroleum ether/ethyl acetate=1:6) yield an additional 1.22 g of product. Yield: 3.51 g (63%); mass spectroscopy [M+H]⁺=194/196.

c) tert-butyl{3-[3-(4-chloro-phenyl)-5-methyl-[1,2,4]triazol-1-yl]-1,1-dimethyl-propyl}-carbamate: 3.48 g (18.0 mmol) 3-(4-chloro-phenyl)-5-methyl-1H-[1.24]triazole and 5.98 g (27.0 mmol) tert-butyl(3-chloro-1,1-dimethyl-propyl)-carbamate are reacted and worked up in the manner described for intermediate product 1c). Yield: 3.89 g (57%); mass spectroscopy [M+H]⁺=379/381.

d) methyl 3-[1-(3-amino-3-methyl-butyl)-5-methyl-1H-[1,2,4]triazol-3-yl]-benzoate: obtained from tert-butyl{3-[3-(4-chloro-phenyl)-5-methyl-[1,2,4]triazol-1-yl]-1,1-dimethyl-propyl}-carbamate according to the method described for intermediate product 1d). Yield: 3.65 g (trifluoroacetate); mass spectroscopy [M+H]⁺=279/281.

### Intermediate Product 7: 1,1-dimethyl-3-[5-methyl-3-(4-trifluoromethyl-phenyl)-[1,2,4]triazol-1-yl]-propylamine

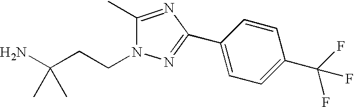

a) 4-trifluoromethyl-benzoic acid N′-(1-imino-ethyl)-hydrazide: 4.78 g (23.4 mmol) 4-(trifluoromethyl)benzoic acid hydrazide and 5.21 g (42.1 mmol) ethylacetimidate hydrochloride are reacted in the manner described for intermediate product 1a). Yield: 6.02 g; mass spectroscopy [M+H]⁺=246.

b) 5-methyl-3-(4-trifluoromethyl-phenyl)-1H-[1,2,4]triazole: prepared from 6.02 g (24.6 mmol) 4-trifluoromethyl-benzoic acid N′-(1-imino-ethyl)-hydrazide analogously to the method described for intermediate product 1b). Yield: 4.76 g (85%); mass spectroscopy [M+H]⁺=228.

c) tert-butyl{1,1-dimethyl-3-[5-methyl-3-(4-trifluoromethyl-phenyl)-[1,2,4]triazol-1-yl]-propyl}-carbamate: The target compound is obtained analogously to the method described for intermediate product 1c) from 4.90 g (21.6 mmol) 5-methyl-3-(4-trifluoromethyl-phenyl)-1H-[1,2,4]triazole and 7.17 g (32.4 mmol) tert-butyl(3-chloro-1,1-dimethyl-propyl)-carbamate. Yield: 5.06 g (57%); mass spectroscopy [M+H]⁺=413.

d) 1,1-dimethyl-3-[5-methyl-3-(4-trifluoromethyl-phenyl)-[1,2,4]triazol-1-yl]-propylamine: Prepared according to the method described for intermediate product 1d) from tert-butyl{1,1-dimethyl-3-[5-methyl-3-(4-trifluoromethyl-phenyl)-[1,2,4]triazol-1-yl]-propyl}-carbamate. Yield: 4.72 g (trifluoroacetate); mass spectroscopy [M+H]⁺=313.

### Intermediate Product 8: 3-(3-benzo[1,3]dioxol-5-yl-5-methyl-[1,2,4]triazol-1-yl)-1,1-dimethyl-propylamine

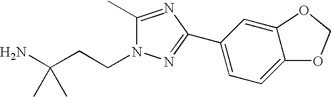

This is prepared analogously to the syntheses described hereinbefore. Mass spectroscopy [M+H]⁺=289.

### Intermediate Product 9: 3-[3-(4-methoxy-phenyl)-5-methyl-[1,2,4]triazol-1-yl]-1,1-dimethyl-propylamine

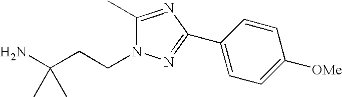

a) 4-methoxy-benzoic acid N′-(1-imino-ethyl)-hydrazide: 4.6 g (0.20 mol) sodium in 200 mL ethanol are combined at ambient temperature with a solution of 25 g (0.20 mol) ethylacetimidate hydrochloride in 200 mL ethanol. The sodium chloride precipitated is suction filtered and 33.2 g (0.20 mol) 4-methoxybenzoic acid hydrazide are added to the filtrate. The reaction mixture is stirred overnight at ambient temperature and then cooled. The precipitate formed is separated off and washed with ethanol and diethyl ether. Yield: 33.6 g (81%); melting range=179-181° C.

b) 3-(4-methoxy-phenyl)-5-methyl-1H-[1,2,4]triazole: 33.6 g (162 mmol) 4-methoxy-benzoic acid N′-(1-imino-ethyl)-hydrazide are heated to 180° C. for 30 minutes. After cooling the residue is dissolved in 250 mL chloroform and extracted repeatedly with aqueous sodium hydroxide solution. The aqueous phases are combined, washed with chloroform, filtered and adjusted to an acidic pH by the addition of glacial acetic acid. The precipitated solid is suction filtered, washed with water and dissolved by heating in chloroform. The solvent is evaporated down and the residue is filtered. The solid is washed with chloroform and diethyl ether. Yield: 23.1 g (75%); melting range=169-171° C.

c) 3-[3-(4-methoxy-phenyl)-5-methyl-[1,2,4]triazol-1-yl]-1,1-dimethyl-propylamine: The target compound is obtained from the reaction of 21.4 g (113 mmol) 3-(4-methoxy-phenyl)-5-methyl-1H-[1,2,4]triazole and 25 g (119 mmol) (3-chloro-1,1-dimethyl-propyl)-[1-phenyl-methylidene]-amine. The product is dissolved in 100 mL acetone and acidified with 8.5 mL 32% aqueous hydrochloric acid and cooled. The hydrochloride precipitated is suction filtered and washed with acetone and diethyl ether. Yield: 20.3 g; melting range=190-194° C.

### Synthesis of the Examples

General method 1: 1 mmol of 6-benzyloxy-8-(2-ethoxy-2-hydroxy-acetyl)-4H-benzo[1,4]oxazin-3-one and 1 mmol amine are stirred for 15 minutes in 5 mL tetrahydrofuran at 60° C. The mixture is cooled to 0° C. and under an argon atmosphere 1.5 mL of a 2 molar solution of lithium borohydride in tetrahydrofuran is added dropwise. The mixture is stirred for 15 min at 0° C., combined with 10 mL dichloromethane and 3 mL water, stirred for another hour at ambient temperature and then filtered through kieselguhr, while eluting with dichloromethane. The eluate is freed from the solvent and the residue is purified by chromatography, if necessary. The benzylether thus obtained is dissolved in methanol and hydrogenated with palladium on charcoal (10%) as catalyst at 2.5 bar and ambient temperature. Then the catalyst is separated off and the crude product is purified by chromatography (reverse phase, acetonitrile/water gradient with 0.1% trifluoroacetic acid).

General method 2: 1 mmol 6-benzyloxy-8-(2-ethoxy-2-hydroxy-acetyl)-4H-benzo[1,4]oxazin-3-one and 1 mmol amine are suspended in 5 mL ethanol and heated to 70° C. The resulting solution is stirred for one hour at 70° C. and then cooled to ambient temperature. After the addition of 113 mg (3 mmol) sodium borohydride the mixture is stirred for 3 hours at ambient temperature, combined with 0.7 mL saturated potassium carbonate solution and stirred for another 30 minutes. It is filtered through aluminium oxide (basic), repeatedly washed with dichloromethane/methanol=15:1, evaporated down and chromatographed (silica gel; dichloromethane with 0-10% methanol:ammonia=9:1). The benzylether thus obtained is dissolved in 10 mL methanol and hydrogenated with palladium on charcoal as catalyst at 1 bar hydrogen pressure. Then the catalyst is filtered off and the filtrate is evaporated down.

### Example 1

### 8-(2-{3-[3-(4-fluoro-phenyl)-5-methyl-[1,2,4]triazol-1-yl]-1,1-dimethyl-propylamino}-1-hydroxy-ethyl)-6-hydroxy-4H-benzo[1,4]oxazin-3-one

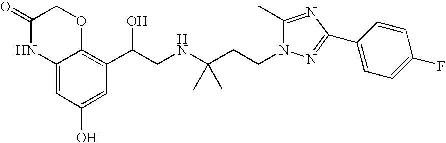

Obtained by reacting 6-benzyloxy-8-(2-ethoxy-2-hydroxy-acetyl)-4H-benzo[1,4]oxazin-3-one and 3-[3-(4-fluoro-phenyl)-5-methyl-[1,2,4]triazol-1-yl]-1,1-dimethyl-propylamine according to General Method 1. The final purification is carried out by chromatography (reverse phase, acetonitrile/water gradient with 0.1% trifluoroacetic acid). Yield: 134 mg (29%, trifluoroacetate); mass spectroscopy [M+H]⁺=470.

### Example 2

### 8-{2-[1,1-dimethyl-3-(5-methyl-3-p-tolyl-[1,2,4]triazol-1yl)-propylamino]-1-hydroxy-ethyl}-6-hydroxy-4H-benzo[1,4]oxazin-3-one

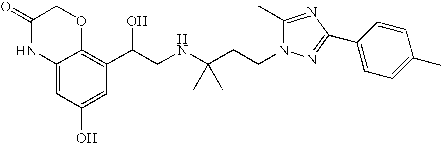

Prepared from 6-benzyloxy-8-(2-ethoxy-2-hydroxy-acetyl)-4H-benzo[1,4]oxazin-3-one and 1,1-dimethyl-3-(5-methyl-3-p-tolyl-[1,2,4]triazol-1-yl]-propylamine according to General Method 1. Yield: 283 mg (49%, trifluoroacetate); mass spectroscopy [M+H]⁺=466.

### Example 3

### 8-(2-{1,1-dimethyl-3-[5-methyl-3-(4-trifluoromethyl-phenyl)-[1,2,4]triazol-1-yl]-propylamino}-1-hydroxy-ethyl)-6-hydroxy-4H-benzo[1,4]oxazin-3-one

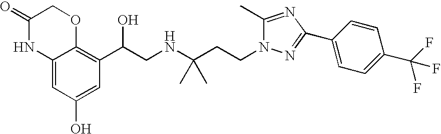

Prepared from 6-benzyloxy-8-(2-ethoxy-2-hydroxy-acetyl)-4H-benzo[1,4]oxazin-3-one and 1,1-dimethyl-3-[5-methyl-3-(4-trifluoromethyl-phenyl)-[1,2,4]triazol-1-yl]-propylamine analogously to General Method 1. Yield: 234 mg (37%, trifluoroacetate); mass spectroscopy [M+H]⁺=520.

### Example 4

### 8-(2-{3-[3-(3,5-difluoro-phenyl)-5-methyl-[1,2,4]triazol-1-yl]-1,1-dimethyl-propylamino}-1-hydroxy-ethyl)-6-hydroxy-4H-benzo[1,4]oxazin-3-one

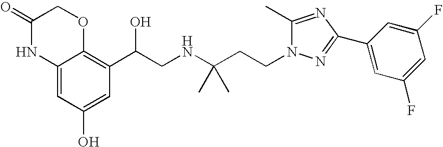

Prepared from 6-benzyloxy-8-(2-ethoxy-2-hydroxy-acetyl)-4H-benzo[1,4]oxazin-3-one and 3-[3-(3,5-difluoro-phenyl)-5-methyl-[1,2,4]triazol-1-yl]-1,1-dimethyl-propylamine according to General Method 1. Yield: 208 mg (35%, trifluoroacetate); mass spectroscopy [M+H]⁺=488.

### Example 5

### 3-(1-{3-[2-hydroxy-2-(6-hydroxy-3-oxo-3,4-dihydro-2H-benzo[1,4]oxazin-8-yl)-ethylamino]-3-methyl-butyl}-5-methyl-1H-[1,2,4]triazol-3-yl-benzoic acid

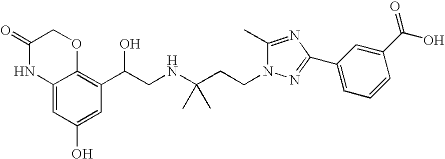

a) methyl 3-(1-{3-[2-(6-benzyloxy-3-oxo-3,4-dihydro-2H-benzo[1,4]oxazin-8-yl)-2-hydroxy-ethylamino}-3-methyl-butyl}-5-methyl-1H-[1,2,4]triazol-3-yl)-benzoate: prepared from 6-benzyloxy-8-(2-ethoxy-2-hydroxy-acetyl)-4H-benzo[1,4]oxazin-3-one and methyl 3-[1-(3-amino-3-methyl-butyl)-5-methyl-1H-[1,2,4]triazol-3-yl]-benzoate analogously to General Method 1. Final purification is carried out by chromatography (reverse phase, acetonitrile/water gradient with 0.1% trifluoroacetic acid). Yield: 550 mg (77%, trifluoroacetate); mass spectroscopy [M+H]⁺=510.

b) 3(1-{3-[2-(6-benzyloxy-3-oxo-3,4-dihydro-2H-benzo[1,4]oxazin-8-yl)-2-hydroxy-ethylamino]-3-methyl-butyl}-5-methyl-1H-[1,2,4]triazol-3-yl)-benzoic acid: a solution of 550 mg (0.72 mmol) methyl 3-(1-{3-[2-(6-benzyloxy-3-oxo-3,4-dihydro-2H-benzo[1,4]oxazin-8-yl)-2-hydroxy-ethylamino]-3-methyl-butyl}-5-methyl-1H-[1,2,4]triazol-3-yl)-benzoate trifluoroacetate in 10 mL methanol is combined with 2 mL of a 2 molar sodium hydroxide solution and refluxed for 30 minutes. After the methanol has been distilled off, 5 mL water, 10 mL n-butanol and 5 mL acetic acid are added. The precipitate formed is suction filtered and washed with diethyl ether. Yield: 300 mg (56%, trifluoroacetate); mass spectroscopy [M+H]⁺=586.

c) 3-(1-{3-[2-hydroxy-2-(6-hydroxy-3-oxo-3,4-dihydro-2H-benzo[1,4]oxazin-8-yl)-ethylamino]-3-methyl-butyl}-5-methyl-1H-[1,2,4]triazol-3-yl-benzoic acid: 250 mg (0.36 mmol) 3-(1-{3-[2-(6-benzyloxy-3-oxo-3,4-dihydro-2H-benzo[1,4]oxazin-8-yl)-2-hydroxy-ethylamino]-3-methyl-butyl}-5-methyl-1H-[1,2,4]triazol-3-yl)-benzoic acid trifluoroacetate are dissolved in 5 mL methanol and hydrogenated in the presence of palladium on charcoal (10%) at ambient temperature and 2.5 bar hydrogen pressure. The catalyst is suction filtered, the filtrate is evaporated down and the residue is purified by chromatography (reverse phase, acetonitrile/water gradient with 0.1% trifluoroacetic acid). Yield: 62 mg (28%, trifluoroacetate); mass spectroscopy [M+H]⁺=496.

### Example 6

### 8-(2-{3-[3-(4-chloro-phenyl)-5-methyl-[1,2,4]triazol-1-yl]-1,1-dimethyl-propylamino}-1-hydroxy-ethyl)-6-hydroxy-4H-benzo[1,4]oxazin-3-one

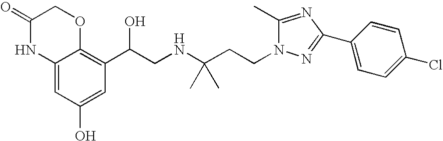

a) 6-benzyloxy-8-(2-{3-[3-(4-chloro-phenyl)-5-methyl-[1,2,4]triazol-1-yl]-1,1-dimethyl-propylamino}-1-hydroxy-ethyl)-4H-benzo[1,4]oxazin-3-one: prepared from 6-benzyloxy-8-(2-ethoxy-2-hydroxy-acetyl)-4H-benzo[1,4]oxazin-3-one and 3-[3-(4-chloro-phenyl)-5-methyl-[1,2,4]triazol-1-yl]-1,1-dimethyl-propylamine analogously to General Method 1. The crude product is purified by chromatography (reverse phase, acetonitrile/water gradient with 0.1% trifluoroacetic acid). Yield: 550 mg (80%, trifluoroacetate); mass spectroscopy [M+H]⁺=576.

8-(2-{3-[3-(4-chloro-phenyl)-5-methyl-[1,2,4]triazol-1-yl]-1,1-dimethyl-propylamino}-1-hydroxy-ethyl)-6-hydroxy-4H-benzo[1,4]oxazin-3-one: 550 mg (0.80 mmol) 6-benzyloxy-8-(2-{3-[3-(4-chloro-phenyl)-5-methyl-[1,2,4]triazol-1-yl]-1,1-dimethyl-propylamino}-4H-benzo[1,4]oxazin-3-one are dissolved in 3 mL dichloromethane and cooled to −78° C. 2 mL of a 1 molar solution of boron tribromide in dichloromethane are added dropwise and the mixture is heated to ambient temperature. It is stirred for 10 minutes at this temperature and then 10 mL dichloromethane and 3 mL water are added and the mixture is stirred for 30 minutes. It is filtered through kieselguhr, during which time it is eluted with dichloromethane and methanol. The eluate is evaporated down and the residue is purified by chromatography (reverse phase, acetonitrile/water gradient with 0.1% trifluoroacetic acid). Yield: 29 mg (6%, trifluoroacetate); mass spectroscopy [M+H]⁺=486/8.

### Example 7

### 8-(2-{3-[5-ethyl-3-(4-methoxy-phenyl)-[1,2,4]triazol-1-yl]-1,1-dimethyl-propylamino}-1-hydroxy-ethyl)-6-hydroxy-4H-benzo[1,4]oxazin-3-one

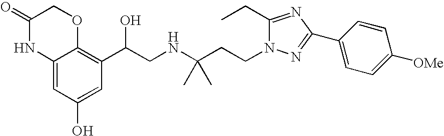

Prepared from 6-benzyloxy-8-(2-ethoxy-2-hydroxy-acetyl)-4H-benzo[1,4]oxazin-3-one and 3-[5-ethyl-3-(4-methoxy-phenyl)-[1,2,4]triazol-1-yl]-1,1-dimethyl-propylamine according to General Method 1. Yield: 267 mg (44%, trifluoroacetate); mass spectroscopy [M+H]⁺=496.

### Example 8

### 6-hydroxy-8-(1-hydroxy-2-{3-[3-(4-methoxy-phenyl)-5-methyl-[1,2,4]triazol-1-yl]-1,1-dimethyl-propylamino}-ethyl)-4H-benzo[1,4]oxazin-3-one

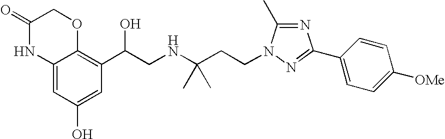

Prepared from 6-benzyloxy-8-(2-ethoxy-2-hydroxy-acetyl)-4H-benzo[1,4]oxazin-3-one and 3-[3-(4-methoxy-phenyl)-5-methyl-[1,2,4]triazol-1-yl]-1,1-dimethyl-propylamine according to General Method 2. Yield: 217 mg (45%); mass spectroscopy [M+H]⁺=482.

### Example 9

### 8-{2-[3-(3-benzo[1,3]dioxol-5-yl-5-methyl-[1,2,4]triazol-1-yl)-1,1-dimethyl-propylamino]-1-hydroxy-ethyl}-6-hydroxy-4H-benzo[1,4]oxazin-3-one

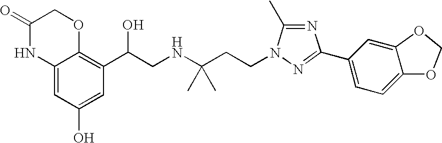

Prepared from 6-benzyloxy-8-(2-ethoxy-2-hydroxy-acetyl)-4H-benzo[1,4]oxazin-3-one and 3-(3-benzo[1,3]dioxol-5-yl-5-methyl-[1,2,4]triazol-1-yl)-1,1-dimethyl-propylamine according to General Method 2. Yield: 236 mg (48%); mass spectroscopy [M+H]⁺=496.

### Example 10

### 7-{2-[3-(3-benzo[1,3]dioxol-5-yl-5-methyl-[1,2,4]triazol-1-yl)-1,1-dimethyl-propylamino]-1-hydroxy-ethyl}-5-hydroxy-3H-benzoxazol-2-one

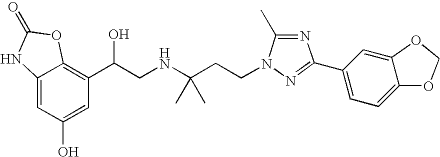

a) 7-acetyl-5-benzyloxy-3H-benzoxazol-2-one: At 20 to 40° C. 52 g (0.53 mol) phosgene are piped into a solution of 121 g (0.47 mol) 1-(3-amino-5-benzyloxy-2-hydroxy-phenyl)-ethanon in 800 mL pyridine. The reaction mixture is heated to 50° C. for 2 hours, then poured onto ice and acidified with conc. hydrochloric acid. The precipitated solid is repeatedly recrystallised from ethanol with the addition of activated charcoal. Yield: 67.5 g (51%); melting range: 163-166° C.

b) 5-benzyloxy-7-(2-ethoxy-2-hydroxy-acetyl)-3H-benzoxazol-2-one: 20 g (71 mmol) 7-acetyl-5-benzyloxy-3H-benzoxazol-2-one and 8 g (72 mmol) selenium dioxide are refluxed in the presence of activated charcoal in 100 mL dioxane and 3.1 mL water for 8 hours with stirring. The solid is filtered off, the solvent is distilled off and the residue is combined with 50 mL ethanol. The mixture is refluxed for 15 minutes and then filtered through activated charcoal. The solid precipitated on cooling is suction filtered after 3 hours and washed with ethanol and diethyl ether. Yield: 7 g (29%); melting range: 140-143° C.

c) 17-{2-[3-(3-benzo[1,3]dioxol-5-yl-5-methyl-[1,2,4]triazol-1-yl)-1,1-dimethyl-propylamino]-1-hydroxy-ethyl}-5-hydroxy-3H-benzoxazol-2-one: 72 mg (0.5 mmol) 5-benzyloxy-7-(2-ethoxy-2-hydroxy-acetyl)-3H-benzoxazol-2-one and 144 mg (0.5 mmol) 3-(3-benzo[1,3]dioxol-5-yl-5-methyl-[1,2,4]triazol-1-yl)-1,1-dimethyl-propylamine are stirred in 8 mL ethanol for 90 minutes at 80° C. After cooling to ambient temperature 19 mg (0.5 mmol) sodium borohydride are added and the mixture is stirred for 2 hours at ambient temperature. It is acidified with 1 N hydrochloric acid, stirred for 10 minutes and then made alkaline with potassium carbonate solution. It is diluted with ethyl acetate and filtered through kieselguhr. The organic phase remaining is evaporated down and the residue is purified by chromatography. The benzylether thus obtained is dissolved in ethanol and hydrogenated with palladium on charcoal (10%) as catalyst at 2.5 bar and ambient temperature. Then the catalyst is separated off and the crude product is purified by chromatography (reverse phase, acetonitrile/water gradient with 0.1% trifluoroacetic acid). Yield: 8 mg (3%, trifluoroacetate); mass spectroscopy [M+H]⁺=482.

### Example 11

### 8-{2-[3-(3-benzo[1,3]dioxol-5-yl-5-methyl-[1,2,4]triazol-1-yl)-1,1-dimethyl-propylamino]-1-hydroxy-ethyl}-6-hydroxy-2,2-dimethyl-4H-benzo[1,4]oxazin-3-one

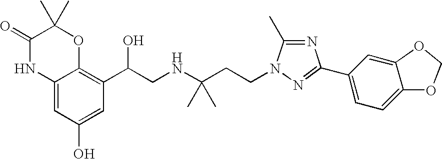

a) N-(3-acetyl-5-benzyloxy-2-hydroxy-phenyl)-2-bromo-2-methyl-propionamide: 4.64 g (25 mmol) 2-bromo-2-methyl-propionyl chloride are added dropwise at 5-20° C. to a solution of 5.15 g (20 mmol) 1-(3-amino-5-benzyloxy-2-hydroxy-phenyl)-ethanone in 20 mL pyridine. After the addition has ended the mixture is stirred for 15 minutes, combined with ice water and 100 mL ethyl acetate and acidified with conc. hydrochloric acid. The organic phase is separated off, washed with water and dried on sodium sulphate. After the solvent has been distilled off the residue is crystallised from a diethyl ether/petroleum ether mixture. Yield: 6.8 g (84%); melting range: 88-90° C.

b) 8-acetyl-6-benzyloxy-2,2-dimethyl-4H-benzo[1,4]oxazin-3-one: 6.60 g (16.2 mmol) N-(3-acetyl-5-benzyloxy-2-hydroxy-phenyl)-2-bromo-2-methyl-propionamide and 2.76 g (20 mmol) potassium carbonate are refluxed for 1 hour in 70 mL acetonitrile with stirring. The solid is suction filtered, the filtrate is evaporated down and the residue is combined with 30 mL ethyl acetate. After further filtration and distillation of the solvent the crude product is crystallised from a little methanol. Yield: 1.00 g (19%); mass spectroscopy [M+H]⁺=326; melting range =148-150° C.

c) 6-benzyloxy-8-(2-ethoxy-2-hydroxy-acetyl)-2,2-dimethyl-4H-benzo[1,4]oxazin-3-one: This is prepared analogously to the method described for Example 10b) from 8-acetyl-6-benzyloxy-2,2-dimethyl-4H-benzo[1,4]oxazin-3-one.

d) 8-{2-[3-(3-benzo[1,3]dioxol-5-yl-5-methyl-[1,2,4]triazol-1-yl)-1,1-dimethyl-propylamino]-1-hydroxy-ethyl}-6-hydroxy-2,2-dimethyl-4H-benzo[1,4]oxazin-3-one: prepared from 385 mg (1 mmol) 6-benzyloxy-8-(2-ethoxy-2-hydroxy-acetyl)-2,2-dimethyl-4H-benzo[1,4]-oxazin-3-one and 402 mg (1 mmol) 3-(3-benzo[1,3]dioxol-5-yl-5-methyl-[1,2,4]triazol-1-yl)-1,1-dimethyl-propylamine according to General Method 1. Yield: 37 mg (6%, trifluoroacetate); mass spectroscopy [M+H]⁺=524.

1. Pharmaceutical combinations which contain in addition to one or more compounds of general formula 1 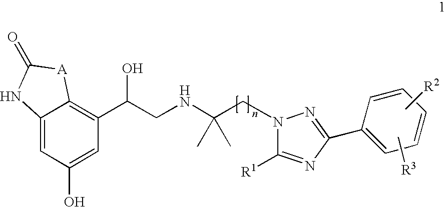
wherein
n denotes 1, 2, 3 or 4;
A denotes a double-bonded group selected from among O, S, NR⁶; CR⁴R⁵—S, CR⁴R⁵—O, CR⁴R⁵—NR⁶, CH═CH or CH₂—CH₂;
R¹ denotes C₁₋₆-alkyl;
R² and R³ which may be identical or different denote H, C₁₋₆-alkyl, C₂₋₆-alkenyl, C₂₋₆-alkynyl, C₃₋₆-cycloalkyl, C₁₋₆-haloalkyl, O—C₁₋₆-haloalkyl, halogen, OH, CN, NO₂, O—C₁₋₆-alkyl, C₂₋₆-alkyl-OH, NH₂, NH—C₁₋₆-alkyl, N(C₁₋₆-alkyl)₂, NHCO—C₁₋₆-alkyl, NHSO₂—C₁₋₆-alkyl, S—C₁₋₆-alkyl, SO—C₁₋₆-alkyl, SO₂—C₁₋₆-alkyl, SO₂NH₂, SO₂NH—C₁₋₆-alkyl, SO₂N(C₁₋₆-alkyl)₂, CONH₂, CONH—C₁₋₆-alkyl, CON(C₁₋₆-alkyl)₂, CO—C₁₋₆-alkyl, COOH or COO—C₁₋₄-alkyl, or
R² and R³ together denote a 2-bonded group selected from O—CR⁴R⁵—O, O—CR⁴R⁵—NR⁶ or CH═CH—CH═CH;
R⁴ denotes H or C₁₋₆-alkyl;
R⁵ denotes H or C₁₋₆-alkyl;
R⁶ denotes H or C₁₋₆-alkyl;
at least one further active substance 2.

2. Pharmaceutical combinations according to claim 1, which contain in addition to one or more compounds of general formula 1 as a further active substance 2 one or more compounds which are selected from among the categories of the anticholinergics (2a), PDE-IV-inhibitors (2b), steroids (2c), LTD4-antagonists (2d) and EGFR-inhibitors (2e).

3. Pharmaceutical combinations according to claim 1, which contain one or more compounds of general formula 1, wherein
n denotes 1, 2 or 3, preferably 2;
A denotes a double-bonded group selected from among CR⁴R⁵—O, CH═CH or CH₂—CH₂, preferably CR⁴R⁵—O;
R¹ denotes C₁₋₄-alkyl;
R² and R³ which may be identical or different denote H, C₁₋₄-alkyl, C₂₋₄-alkenyl, C₂₋₄-alkynyl, C₃₋₆-cycloalkyl, C₁₋₄-haloalkyl, O—C₁₋₄-haloalkyl, halogen, OH, CN, NO₂, C₂₋₄-alkyl-OH, O—C₁₋₄-alkyl, COOH or COO—C₁₋₄-alkyl, or
R² and R³ together denote a 2-bonded group selected from O—CR⁴R⁵—O, O—CR⁴R⁵—NR⁶ or CH═CH—CH═CH;
R⁴ denotes H or C₁₋₄-alkyl;
R⁵ denotes H or C₁₋₄-alkyl;
R⁶ denotes H or C₁₋₄-alkyl.

4. Pharmaceutical combinations according to claim 1, which contain one or more compounds of general formula 1, wherein
A denotes a double-bonded group selected from among CR⁴R⁵—O, CH═CH or CH₂—CH₂, preferably CR⁴R⁵—O wherein
R⁴ denotes H, methyl, ethyl, preferably H or methyl, particularly preferably H;
R⁵ denotes H, methyl, ethyl, preferably H or methyl, particularly preferably H.

5. Pharmaceutical combinations according to claim 1, which contain one or more compounds of general formula 1 in the form of the individual optical isomers, mixtures of the individual enantiomers or racemates.

6. Pharmaceutical combinations according to claim 1, which contain one or more compounds of general formula 1 in the form of the acid addition salts with pharmacologically acceptable acids and optionally in the form of the solvates and/or hydrates thereof.

7. Pharmaceutical combinations according to claim 1, which contain an anticholinergic (2a) as a further active substance 2 in addition to one or more compounds of general formula 1.

8. Pharmaceutical combinations according to claim 1, which contain a PDE IV-Inhibitor (2b) as a further active substance 2 in addition to one or more compounds of general formula 1.

9. Pharmaceutical combinations according to claim 1, which contain a steroid (2c) as a further active substance 2 in addition to one or more compounds of general formula 1.

10. Pharmaceutical combinations according to claim 1, which contain an LTD4-antagonist (2d) as a further active substance 2 in addition to one or more compounds of general formula 1.

11. Pharmaceutical combinations according to claim 1, which contain an EGFR-inhibitor (2e) as a further active substance 2 in addition to one or more compounds of general formula 1.

12. Pharmaceutical combinations according to claim 1, characterised in that they also contain, in addition to therapeutically effective amounts of 1, therapeutically effective amounts of an anticholinergic (2a) as well as therapeutic amounts of a PDEIV inhibitor (2b).

13. Pharmaceutical combinations according to claim 1, characterised in that they also contain, in addition to therapeutically effective amounts of 1, therapeutically effective amounts of an anticholinergic (2a), as well as therapeutic amounts of a steroid (2c).

14. Pharmaceutical combinations according to claim 1, characterised in that they also contain, in addition to therapeutically effective amounts of 1, therapeutically effective amounts of an anticholinergic (2a), as well as therapeutic amounts of an LTD4-antagonist (2d).

15. Pharmaceutical combinations according to claim 1, characterised in that they also contain, in addition to therapeutically effective amounts of 1, therapeutically effective amounts of an anticholinergic (2a), as well as therapeutic amounts of an EGFR inhibitor (2e).

16. Pharmaceutical combinations according to claim 1, characterised in that they also contain, in addition to therapeutically effective amounts of 1, therapeutically effective amounts of a PDEIV inhibitor (2b), as well as therapeutic amounts of a steroid (2c).

17. Pharmaceutical combinations according to claim 1, characterised in that they also contain, in addition to therapeutically effective amounts of 1, therapeutically effective amounts of a PDEIV inhibitor (2b), as well as therapeutic amounts of an LTD4-antagonist (2d).

18. Pharmaceutical combinations according to claim 1, characterised in that they also contain, in addition to therapeutically effective amounts of 1, therapeutically effective amounts of a PDEIV inhibitor (2b), as well as therapeutic amounts of an EGFR inhibitor (2e).

19. Pharmaceutical combinations according to claim 1, characterised in that they also contain, in addition to therapeutically effective amounts of 1, therapeutically effective amounts of a steroid (2c), as well as therapeutic amounts of an LTD4-antagonist (2d).

20. Pharmaceutical combinations according to claim 1, characterised in that they also contain, in addition to therapeutically effective amounts of 1, therapeutically effective amounts of a steroid (2c), as well as therapeutic amounts of an EGFR inhibitor (2e).

21. Pharmaceutical combinations according to claim 1, characterised in that they also contain, in addition to therapeutically effective amounts of 1, therapeutically effective amounts of an LTD4-antagonist (2d), as well as therapeutic amounts of an EGFR inhibitor (2e).

22. Pharmaceutical combinations according to claim 1, characterised in that in addition to therapeutically effective amounts of 1 and 2, they also contain a pharmaceutically acceptable carrier.

23. Pharmaceutical combinations according to claim 1, characterised in that they contain no pharmaceutically acceptable carrier in addition to therapeutically effective amounts of 1 and 2.

24. Pharmaceutical combination according to claim 1, characterised in that it is in the form of a formulation suitable for inhalation.

25. Pharmaceutical combination according to claim 24, characterised in that it is a preparation selected from the group comprising inhalable powders, propellant-driven metered-dose aerosols and propellant-free inhalable solutions or suspensions.

26. Pharmaceutical combination according to claim 25, characterised in that the preparation is an inhalable powder which contains 1 and 2 in admixture with suitable physiologically acceptable excipients selected from the group comprising monosaccharides, disaccharides, oligo- and polysaccharides, polyalcohols, salts, or mixtures of these excipients with one another.

27. Pharmaceutical combination according to claim 25 characterised in that the preparation is a propellant-driven inhalable aerosol which contains 1 and 2 in dissolved or dispersed form.

28. Pharmaceutical combination according to claim 27, characterised in that the inhalable aerosol contains as the propellant gas hydrocarbons such as n-propane, n-butane or isobutane or halohydrocarbons such as chlorinated and/or fluorinated derivatives of methane, ethane, propane, butane, cyclopropane or cyclobutane.

29. Pharmaceutical combination according to claim 28, characterised in that the propellant gas is TG11, TG12, TG134a, TG227 or mixtures thereof, preferably TG134a, TG227 or a mixture thereof.

30. Pharmaceutical combination according to claim 25, characterised in that the preparation is a propellant-free inhalable solution or suspension which contains as solvent water, ethanol or a mixture of water and ethanol.

31. A method for the treatment of inflammatory and obstructive respiratory complaints, for inhibiting premature labour in midwifery (tocolysis), for restoring sinus rhythm in the heart in atrioventricular block, for correcting bradycardic heart rhythm disorders (antiarrhythmic), for treating circulatory shock (vasodilatation and increasing the heart volume) as well as for the treatment of skin irritations and inflammation comprising administering to a patient a pharmaceutical combination according to claim 1.

32. A method for the treatment of inflammatory and obstructive respiratory complaints, for inhibiting premature labour in midwifery (tocolysis), for restoring sinus rhythm in the heart in atrioventricular block, for correcting bradycardic heart rhythm disorders (antiarrhythmic), for treating circulatory shock (vasodilatation and increasing the heart volume) as well as for the treatment of skin irritations and inflammation, comprising administering to a patient a compound of formula 1 according to claim 1, in combination with at least one additional active substance 2.

33. The method according to claim 31, wherein said method is for the treatment of respiratory complaints selected from the group comprising obstructive pulmonary diseases of various origins, pulmonary emphysema of various origins, restrictive pulmonary diseases, interstitial pulmonary diseases, cystic fibrosis, bronchitis of various origins, bronchiectasis, ARDS (adult respiratory distress syndrome) and all forms of pulmonary oedema.

34. The method according to claim 32 wherein said method is for the treatment of respiratory complaints selected from the group comprising obstructive pulmonary diseases of various origins, pulmonary emphysema of various origins, restrictive pulmonary diseases, interstitial pulmonary diseases, cystic fibrosis, bronchitis of various origins, bronchiectasis, ARDS (adult respiratory distress syndrome) and all forms of pulmonary oedema.
